(19) United States
(12) Patent Application Publication
(10) Pub. No.: US 2026/0315474 A1
(43) Pub. Date: Oct. 15, 2026

(54) USE OF MILVEXIAN FOR TREATING OR PREVENTING ADVERSE CEREBROVASCULAR EVENTS OR ADVERSE CARDIOVASCULAR EVENTS IN PATIENTS HAVING ACUTE CORONARY SYNDROME

(71) Applicants: BRISTOL-MYERS SQUIBB COMPANY, Princeton, NJ (US); JANSSSEN PHARMACEUTICA NV, Beerse (BE)

(72) Inventors: Danshi LI, Princeton, NJ (US); Christopher NESSEL, Bridgewater, NJ (US); Alexei PLOTNIKOV, Bridgewater, NJ (US); Elliot BARNATHAN, Spring House, PA (US); Puneet MOHAN, Princeton, NJ (US); Jay HORROW, Princeton, NJ (US)

(21) Appl. No.: 19/476,268

(22) PCT Filed: Apr. 19, 2024
(86) PCT No.: PCT/US2024/025394

§ 371 Date: Oct. 17, 2025

Related U.S. Application Data
(60) Provisional application No. 63/497,111, filed on Apr. 19, 2023.

(51) Int. Cl. A61K 31/513; A61K 31/4365; A61K 31/519; A61K 31/616; A61P 9/00; A61P 9/10

(52) U.S. Cl. CPC A61K 31/513; A61K 31/4365; A61K 31/519; A61K 31/616; A61P 9/00; A61P 9/10

(57) ABSTRACT

A Factor XIa inhibitor having therapeutic properties useful in methods for of preventing adverse cerebrovascular events or adverse cardiovascular events in human patients with acute coronary syndrome.

### CROSS-REFERENCE TO RELATED APPLICATION

This application claims the benefit of U.S. Provisional Application No. 63/497,111, filed Apr. 19, 2023, the entirety of which is incorporated by reference herein.

### TECHNICAL FIELD

The disclosure pertains to the use of milvexian for treating or preventing adverse cerebrovascular events or adverse cardiovascular events in human patients with acute coronary syndrome (ACS) without significantly impairing normal blood clotting process.

### BACKGROUND

Cardiovascular disease (CVD) remains the leading cause of death globally. Despite the availability of current therapies, stroke continues to be a leading cause of death and disability worldwide. In 2019 alone, it resulted in 143 million disability-adjusted life-years and 6.55 million deaths, contributing to a significant portion of healthcare expenditure in Western countries. In the United States, an estimated 805,000 new and recurrent myocardial infarctions (MIs) occur per year. After experiencing acute coronary syndrome (ACS), patients remain at heightened risk. The risk of recurrent cardiovascular (CV) events such as myocardial infarction), ischemic stroke, and CV death in acute MI survivors remains unacceptably high at more than 5% in the first year despite progress with newer thin strut stents/polymers, antithrombotic therapies with aspirin and/or potent P2Y12 inhibitors, widespread application of surgical and percutaneous revascularizations, and the targeted control of modifiable CV risk factors. Recurrent cardiovascular events occur in 5% of acute MI survivors by the end year one, and 60% of those events occur in the first 90 days. Atherosclerosis-associated cardiovascular complications remain the leading cause of morbidity and mortality in the Western world and developing countries. (Katan et al. Global burden of stroke, SeminNeurol., 2018, vol. 38, pp. 208-211).

Milvexian (BMS-986177/JNJ-70033093) is a direct-acting, high-affinity inhibitor of human coagulation FXIa. (Dilger et al., Discovery of milvexian, a high-affinity, orally bioavailable inhibitor of factor XIa in clinical studies for antithrombotic therapy. J Med Chem 2022; 65(3):1770-85). Milvexian is a macrocyclic compound having the structure of Formula (I):

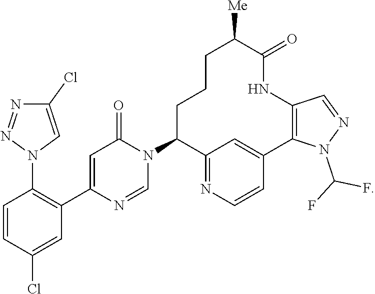 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

Milvexian is also known by its chemical name (5R,9S)-9-(4-(5-chloro-2-(4-chloro-1H-1,2,3-triazol-1-yl)phenyl)-6-oxopyrimidin-1(6H)-yl)-2¹-(difluoromethyl)-5-methyl-2¹H-3-aza-1(4,2)-pyridina-2(5,4)-pyrazolacyclonaphan-4-one.

Milvexian and a method of preparing milvexian are described in U.S. Pat. No. 9,453,018, which is hereby incorporated by reference in its entirety. Solvates, crystalline forms, and amorphous forms of milvexian are also known in the art. See, e.g., WO2021207659 and WO2022081473. An amorphous solid dispersion composition of milvexian in one or more polymers has been described in WO2020210629, which is hereby incorporated by reference in its entirety.

4,114 participants have been included and exposed to the study intervention (milvexian, placebo, or comparator) in the milvexian clinical program. Of the 4,114 participants, 3,229 received milvexian, of which 660 participants were exposed to milvexian in the Phase 1 studies and 2,569 participants were exposed to milvexian in the Phase 2 and 2a studies. Four types of serious bleeding in the milvexian clinical development program were assessed as adverse drug reactions: gastrointestinal bleeding, procedural hemorrhage, nervous system disorder bleeding (hemorrhagic transformation of ischemic stroke and subdural hematoma) and hematuria. The result of Phase II clinical trial of milvexian in patients undergoing TKR (total knee replacement) was published in 2021. (Weitz, et al., Milvexian for the Prevention of Venous Thromboembolism, N. Engl. J. Med. 2021, vol. 385, pp. 2161-2172.) The results of Phase II clinical trial using milvexian in addition to a single or dual antiplatelet therapy (SAPT/DAPT) for preventing non-cardioembolic, non-lacunar ischemic stroke, was published in 2023 (Sharma et al., Safety and efficacy of factor XIa inhibition with milvexian for secondary stroke prevention (AXIOMATIC-SSP): a phase 2, international, randomised, double-blind, placebo-controlled, dose-finding trial, The Lancet Neurology, 2023, vol. 23, pp-46-59).

Thus, vascular and thromboembolic diseases remain the leading causes of death and disability worldwide. Despite significant advances in antithrombotic treatment, anticoagulation therapy has been limited by bleeding and high thrombotic risk remains for many patients.

### SUMMARY

This disclosure provides treatment regimen comprising an oral formulation of milvexian or pharmaceutically acceptable salt or solvate thereof, a potent and highly selective FXIa inhibitor as described below, for targeted modulation of coagulation through inhibition of activated Factor XIa (FXIa), and one or more antiplatelet therapy to treat and prevent thrombus formation and embolism, thus reducing the risks of adverse cerebrovascular or cardiovascular events such as cardiovascular death, myocardial infarction, or stroke in patients with a history of acute coronary syndrome and/or atherosclerotic vascular diseases, without impairing hemostasis by reducing thrombin generation as the result of FXIa inhibition goes hand-in hand with inhibition of platelet activation. The treatment regimen as described herein is given to patients at risk of developing thromboembolic disease to prevent formation of an occlusive thrombus (primary prevention). In some embodiments, the treatment regimen as described herein is given to patients for secondary prevention, following an initial thrombotic episode, for example, secondary prevention of cardiovascular events in patients with a history of acute myocardial infarction or acute coronary syndrome. The treatment regimen as described herein provides a more favorable balance between ischemia and bleeding risk as compared with rivaroxaban or other direct oral anticoagulants (DOACs) for ACS patients. Not only could the treatment regimen block coagulation activation on stents, but could also prevent FXI-mediated thrombus stabilization and growth at sites of vascular injury.

In another aspect, the methods of the disclosure provide a novel treatment regimen comprising a novel immediate release oral tablet comprising milvexian and standard of care antiplatelet therapy for anticoagulation in patients after ACS and at increased risk of bleeding. The methods of the disclosure introduce an opportunity to use FXIa inhibitors in areas or diseases where anticoagulants are underused or not used.

In some aspects, the disclosure provides methods of preventing adverse cerebrovascular events or adverse cardiovascular events in a human patient with acute coronary syndrome, wherein the method comprises administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combination thereof, wherein the pharmaceutical composition is administered twice daily.

In other aspects, the disclosure provides methods for preventing adverse cardiovascular events in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily; and wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb ischemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither.

In other aspects, the disclosure provides methods for reducing incidence rate of one or more of adverse thrombotic event selected from new ischemic stroke, MI, or all-cause death in a human patient diagnosed with acute coronary syndrome, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

In yet other aspects, the disclosure provides methods for preventing ischemic stroke in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof, where the pharmaceutical composition is administered twice daily.

In some aspects of the disclosed methods, the milvexian (or a pharmaceutically acceptable salt or solvate thereof) is orally administered as a solid pharmaceutical composition comprising milvexian (or a pharmaceutically acceptable salt or solvate thereof) and a pharmaceutically acceptable excipient.

In other aspects of the disclosed methods, the administration does not result in a statistically significant increase in major bleeding complications.

### BRIEF DESCRIPTION OF THE DRAWINGS

FIG. 1 shows coagulation pathways. Legend: FXII, factor XII; FXIIa, activated factor XII; FXI, factor XI; FXIa, activated factor XI; FIX, factor IX; FIXa, activated factor IX; FVIIa, activated factor VII; FVII, factor VII; FX, factor X; FXa, activated factor X. See, Kakkar et al., FXI inhibition: The Holy Grail of Haemostasis-Sparing Anticoaulation, EMJ, 2021, vol. 6, pp. 12-20; and Fredenburgh et al., FXIa as a Target for New Anticoagulants, Hamostaseologie, 2021, vol. 41, pp. 104-110.

FIG. 2 activated partial thromboplastin time as set forth in Example 2. The mean (±SD) percent change from baseline aPTT versus time by treatment is presented in FIG. 2. A dose-dependent increase in aPTT percent change from baseline was observed with milvexian over the dose range from 25 mg QD to 200 mg BID. Summary statistics for aPTT measurements and percent change from baseline in the PD population is provided in Table 5. Nominal time points from the prior dose of milvexian were used for PD biomarker data analysis. The 4-hour time point includes all tests collected from 0.5 to 6 hours, the 12-hour time point includes all tests collected from 6 to 12 hours, and the 24-hour time point includes all tests collected from 12 to 72 hours.

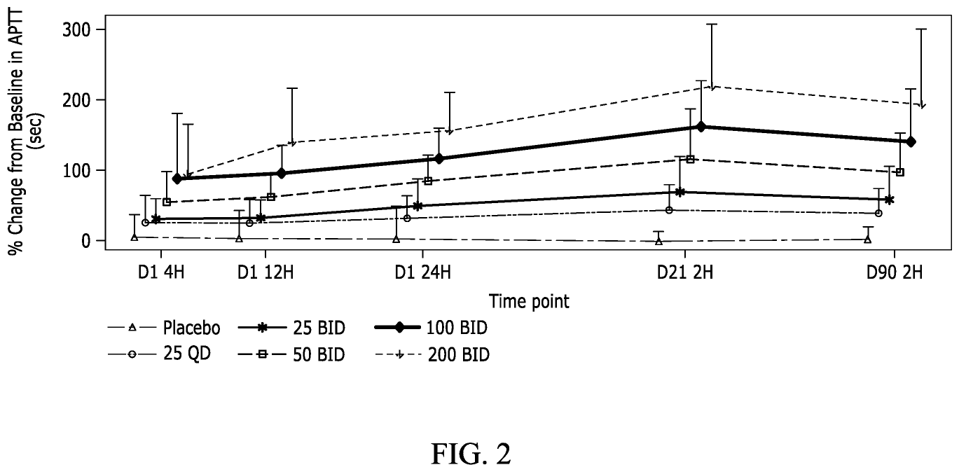

FIG. 3 shows a Kaplan-Meier Plot of Time to Ischemic Stroke and Undetermined Stroke—All Randomized Subjects.

FIG. 4 shows Kaplan-Meier Plot of Time to Ischemic Stroke and Undetermined Stroke—All Randomized Subjects.

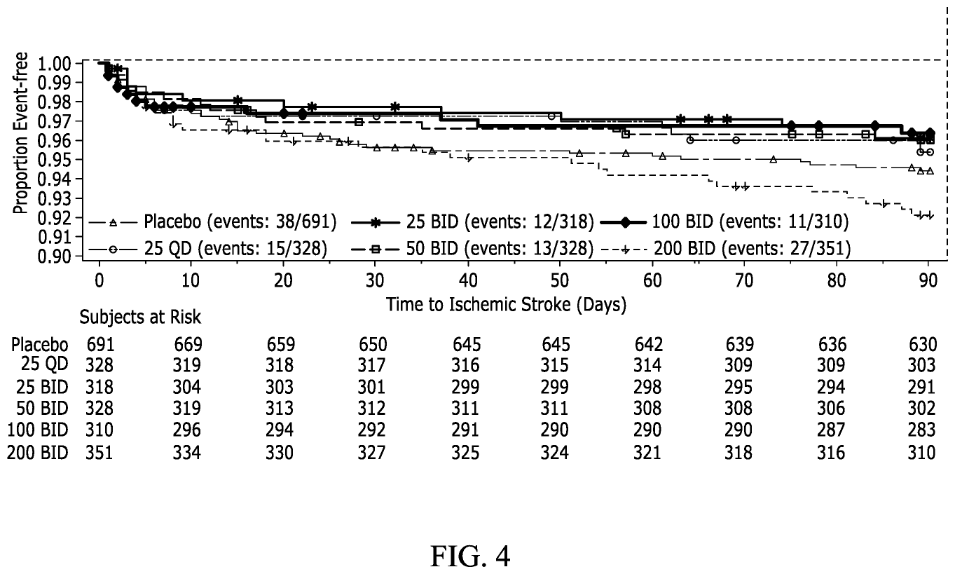

FIG. 5A shows the Day 1 milvexian plasma concentration as a function of time after BID administration of a film-coated direct compression tablet (2×100 mg) of the disclosure compared to the Day 1 milvexian plasma concentration as a function of time after BID administration of a milvexian-containing capsule (2×100 mg). See Example 3.

FIG. 5B shows the Day 5 milvexian plasma concentration as a function of time after BID administration of a film-coated direct compression tablet (2×100 mg) of the disclosure compared to the Day 5 milvexian plasma concentration as a function of time after BID administration of a milvexian-containing capsule (2×100 mg). See Example 3.

FIG. 5C shows the Day 1 milvexian plasma concentration as a function of time after BID administration of a film-coated direct compression tablet (1×25 mg) of the disclosure compared to the Day 1 milvexian plasma concentration as a function of time after BID administration of a milvexian-containing capsule (1×25 mg). See Example 3.

FIG. 5D shows the Day 5 milvexian plasma concentration as a function of time after BID administration of a film-coated direct compression tablet (1×25 mg) of the disclosure compared to the Day 5 milvexian plasma concentration as a function of time after BID administration of a milvexian-containing capsule (1×25 mg). See Example 3.

### DETAILED DESCRIPTION OF ILLUSTRATIVE EMBODIMENTS

In one aspect, the methods of the disclosure provide a novel treatment regimen comprising a novel immediate release oral tablet comprising milvexian for anticoagulation in patients after ACS and at increased risk of bleeding.

In some aspects, the disclosure provides methods of preventing adverse cerebrovascular events or adverse cardiovascular events in a human patient with acute coronary syndrome, wherein the method comprises administering to the human patient a regimen comprising 25 mg of milvexian twice daily.

In some aspects, the disclosure provides methods of preventing adverse cerebrovascular events or adverse cardiovascular events in a human patient with acute coronary syndrome, wherein the method comprises administering to the human patient a regimen comprising a pharmaceutical composition comprising 25 mg of milvexian (or pharmaceutically acceptable salt or solvate thereof), wherein the pharmaceutical composition is administered twice daily.

In some aspects, the disclosure provides methods of preventing adverse cerebrovascular events or adverse cardiovascular events in a human patient with acute coronary syndrome, wherein the method comprises administering to the human patient a regimen comprising 25 mg of milvexian twice daily and (ii) an antiplatelet therapy.

In some aspects, the disclosure provides methods of preventing adverse cerebrovascular events or adverse cardiovascular events in a human patient with acute coronary syndrome, wherein the method comprises administering to the human patient a regimen comprising: (i) a pharmaceutical composition comprising 25 mg of milvexian (or pharmaceutically acceptable salt or solvate thereof); and (ii) an antiplatelet therapy, wherein the pharmaceutical composition is administered twice daily.

In other aspects, the disclosure provides methods for preventing adverse cardiovascular events in a human patient diagnosed with acute coronary syndrome, wherein the method comprises administering to the human patient a regimen comprising about 25 mg of milvexian twice daily; and wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb ischemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither.

In other aspects, the disclosure provides methods for reducing incidence rate of one or more of adverse thrombotic event selected from new ischemic stroke, MI, or all-cause death in a human patient diagnosed with acute coronary syndrome, wherein the method comprises administering to the human patient a regimen comprising about 25 mg to about 100 mg of milvexian twice daily.

In yet other aspects, the disclosure provides methods for preventing ischemic stroke in a human patient diagnosed with acute coronary syndrome, wherein the method comprises administering to the human patient a regimen comprising 25 mg of milvexian twice daily.

This disclosure provides methods for preventing adverse cerebrovascular events or adverse cardiovascular events in a human patient with acute coronary syndrome (ACS), wherein the method comprises administering to the human patient a regimen comprising: (i) milvexian at 25.0 mg twice daily; and (ii) an antiplatelet therapy. In some embodiments, the antiplatelet therapy comprises single antiplatelet therapy (SAPT). In some embodiments, the antiplatelet therapy comprises dual antiplatelet therapy (DAPT). In some embodiments, the antiplatelet therapy selected from aspirin or an P2Y12 inhibitor. In some embodiments, the antiplatelet therapy comprises administering DAPT containing aspirin and clopidogrel for 21 days followed by aspirin monotherapy.

The use of the milvexian treatment in the methods as described in Example 1 below focus on different patient populations from those in the two Phase II clinical trials (AXIOMATIC-TKR and AXIOMATIC-SSP), for example, the treatment methods as described herein are performed on patients with acute coronary syndrome (ACS) for the prevention of adverse cardiovascular events and the pathophysiology of ACS involves coronary atherosclerosis (e.g., arterial atherosclerotic plaque in the heart), whereas the AXIOMATIC-TKR trial was performed on patients post total knee replacement surgery for preventing the development of venous thromboembolism and the AXIOMATIC-SSP trial was performed on patients who had ischemic stroke caused by atherothrombosis associated with cerebrovascular atherosclerosis (e.g., intracranial or cervical arterial atherosclerotic plaque inside the brain, see Sharma et al. supra,) for secondary stroke prevention.

Atherosclerosis is a chronic inflammatory disease of the arterial wall and is characterized by endothelial dysfunction leading to lipid deposition, calcium accumulation in the subendothelial space, followed by leukocyte infiltration and smooth muscle proliferation. Vessel injury, such as occurring during erosion or rupture of atherosclerotic lesions triggers blood coagulation, in attempt to maintain hemostasis (protect against bleeding). This results in thrombus formation and acute coronary syndromes (ACS). Atherothrombosis occurs as a consequence of the production of a thrombus on a rupture or erosive atherosclerotic plaque and clinically manifested as myocardial infarction, (MI) peripheral artery disease (PAD), or ischemic stroke (not due to embolization from the heart) (Moliterno et al. Eds., The ESC Textbook of Thrombosis, Chapter 1, Arterial Thrombosis: Pathophysiological background, pp. 3-12, Oxford University Press, Oxford, 2023).

The link between coagulation and atherothrombosis has been demonstrated in numerous animal and clinical studies (Ngo et al., Pharmacological targeting of coagulation factor XI mitigates the development of experimental atherosclerosis in low-density lipoprotein receptor-deficient mice, J Thromb Haemost. 2021; 19:1001-1017). Numerous coagulation components are present in human atherosclerotic lesions, strongly indicative of roles of coagulation activity during early plaque development. Furthermore, thrombin, tissue factor pathway inhibitor, coagulation factor (F) VIII, and activated FX (FXa) have all been shown to contribute to plaque development in mice. Components of the intrinsic pathway, such as factor XII (FXII) and more recently factor XI (FXI), have also been shown to play a role in atherogenesis in ApoE−/− mice through unclear mechanisms.

Acute coronary syndrome (ACS) is a group of diseases associated with sudden, reduced blood flow to the heart. A blood clot is the most common cause of the reduced blood flow which can lead to other complications (known as major adverse cardiovascular events) such as another heart attack, a stroke, or even death. The pathophysiology of ACS involves arterial thrombus formation, which is comprised of platelet activation and fibrin formation. The mechanism underlying thrombus formation in acute coronary syndrome (ACS) involves both platelets and thrombin. Among patients with ACS, thrombin generation is more rapid and readily amplified than in patients with stable coronary artery disease or in healthy controls. The persistent increase in thrombin generation in both the immediate and chronic phase following ACS has been linked to recurrent events. Thrombin generation remains elevated for year following an ACS, and elevated measures of thrombin generation are associated with higher events rates. While both pathways are targeted in acute care, platelet inhibition has been predominantly administered in the chronic phase, yet thrombin plays a key role in platelet activation and fibrin formation. Among ACS patients, there is also a persistent chronic increase in thrombin generation, which is associated with a higher rate of adverse events (Bahit et al., Thrombin as target for prevention of recurrent events after acute coronary syndromes, Thrombosis Research, 2024, Vol. 235, pp. 116-121).

FXI is activated predominantly by FXIIa and also by thrombin and about 15% of thrombin is generated via the FXIa-mediated mechanism. It has been demonstrated that elevated FXIa is associated with ischemic stroke, venous thromboembolism and ACS. FXIa and circulating active tissue factor (TF) have been detected in individuals with both stable CAD (76% and 6%, respectively) and ACS with a symptom duration <12 h (96% and 38%), respectively, as well as those with heart failure (Zabczyk, et al., Active tissue factor and activated factor XI in circulating blood of patients with systolic heart failure due to ischemic cardiomyopathy, Pol. Arch. Med. Wewn., 120 (2010), pp. 334-340). The SMILE study found a positive association between plasma FXIa and ACS (Doggen et al., Levels of intrinsic coagulation factors and the risk of myocardial infarction among men: opposite and synergistic effects of factors XI and XII, Blood, 108 (2006), pp. 4045-4051). Paszek et al. reported that active factor XI is associated with the risk of cardiovascular events in stable coronary artery disease patients (Atherosclerosis, 2022, vol. 346, pp. 124-132). Factor XIa and active TF were present in 32.6% and 14.7%, respectively, of patients with acute ischemic stroke (Goldman, et al. Activation of blood coagulation and thrombin generation in acute ischemic stroke treated with rtPA, J. Thromb. Thrombolysis, 44 (2017), pp. 362-370). Circulating TF and FXIa were associated with unfavorable long-term outcomes in patients with ischemic cerebrovascular events (Undas et al., Circulating activated factor XI and active tissue factor as predictors of worse prognosis in patients following ischemic cerebrovascular events Thromb. Res., 128 (2011), pp. e62-66).

Increased activity of plasma FXII, FXI, or kallikrein has been associated with atherosclerosis and myocardial infarction, whereas severe FXI deficiency has been associated with reduced risk of stroke and deep vein thrombosis. Hence, it is possible that sustained FXIa generation enhances atherosclerosis, which may also contribute to increased risk of MI, stroke and CV death.

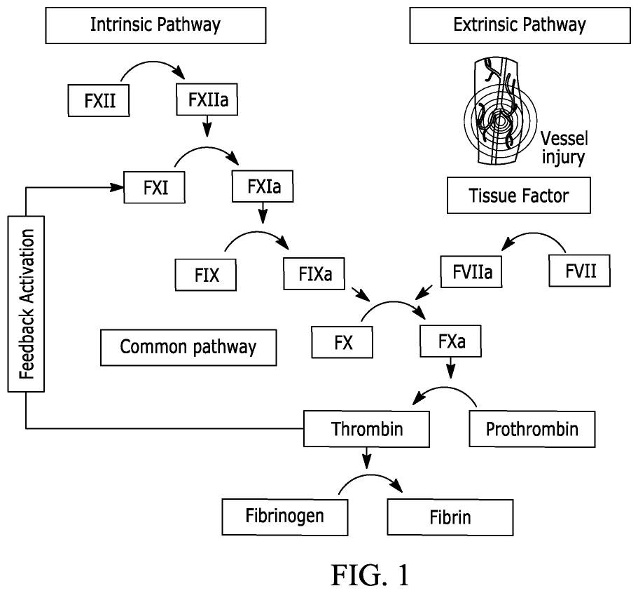

The rupture or erosion of atherosclerotic plaque in a coronary artery triggers platelet activation and aggregation and exposes tissue factor, which initiates the coagulation cascade and leads to thrombin generation and fibrin formation. A critical step in the amplification of thrombin generation is feedback activation of Factor XI by thrombin (FIG. 1). Fibrin and aggregated platelets form a thrombus, which occludes coronary artery blood flow, thereby triggering an ischemic event.

It has recently become clear that patients with CAD often have a generalized atherosclerotic process and more than one third of such patients may also suffer from peripheral artery disease (“PAD”). It is estimated that despite the current standard-of-care therapies, patients post-ACS may have a rate of major adverse cardiovascular event (“MACE”) of approximately 5% in the first year and an additional approximately 0.5% to 1% of highly meaningful additional events such as major adverse limb event (MALE) or symptomatic venous thromboembolism (VTE). The term MAVE, major adverse vascular events, covers the many types of arterial and vascular events that anticoagulants may help prevent.

Thrombin is the most powerful activators of platelets. For the current antithrombotic measures to prevent recurrent thromboembolic events after an ACS, aspirin and P2Y12 inhibitors remain the cornerstone antiplatelet therapies. However, neither aspirin, nor P2Y12 inhibitor blocks activation of platelet by thrombin. The mainstay of CAD treatment is dual antiplatelet therapy with aspirin and one of the P2Y12-receptor inhibitors (clopidogrel, ticagrelor, or prasugrel). A pooled analysis of data from all randomized trials of aspirin versus control for secondary prevention of ACS or chronic CAD confirmed that aspirin reduces the risk and severity of early and late recurrent MACE (Antithrombotic Trialists' Collaboration 2002). Similarly, in participants with atherosclerosis, including a subgroup with MI within 35 days prior to randomization, chronic clopidogrel or aspirin monotherapy yield comparable results (CAPRIE Steering Committee 1996). Despite improvements in invasive and medical management of patients with CAD, recurrent thrombotic events after an ACS occur at unacceptably high rates of 5% or more per year.

In the setting of post-ACS care with rivaroxaban, targeting Factor Xa has been associated with decreased thrombin generation and reduced cardiovascular events, but has been associated with increased bleeding risks. Among the DOACs, rivaroxaban is the only one to successfully undergo phase III evaluation in ACS patients in combination with dual antiplatelet therapy. It reduced the risk of death from cardiovascular causes, myocardial infarction, and stroke, but increased the risk of major bleeding. The COMPASS trial in subjects with stable atherosclerotic vascular disease demonstrated that a dual pathway mechanism with antiplatelet therapy and an anticoagulant improves efficacy by reducing the risk of ischemic stroke with an added major bleeding liability (Eikelboom et al.; COMPASS Investigators. Rivaroxaban with or without Aspirin in Stable Cardiovascular Disease. N Engl J Med 2017; 377:1319-30).

The high human and financial cost of thromboembolic events underscore the need for newer and better therapeutic options for the management of thrombotic disorders. The challenge is in developing an agent that has potent antithrombotic effects but minimal bleeding risk, as it requires blocking thrombotic processes with minimal impact on hemostatic processes. Although many treatments are available for ACS, new therapeutic options such as oral formulation of FXIa inhibitors are needed for preventing the recurrent adverse vascular events in patients with ACS.

Activated coagulation Factor XIa (FXIa) is a key serine protease involved in the amplification of thrombin production. Increased thrombin level brings the risk of thrombosis. Inhibition of FXIa reduces thrombin generation and prevents thrombosis, while preserving normal hemostasis. While FXIa makes relatively modest contributions to hemostasis, it is involved in thrombus growth and stabilization (see FIG. 1). Fredenburgh J C, Weitz J I. Factor XI as a target for new anticoagulants. Hamostaseologie 2021; 41(2):104-10. Human coagulation factor (F) XI deficiency protects against venous thrombosis, stroke, and heart attack, whereas deficiency of other contact pathway components including FXII, plasma prekallikrein, or kininogen do not protect against thrombosis. FXI deficiency, inhibition of FXI production, activated FXI (FXIa) inhibitors, and antibodies to FXI that interfere with FXI/FXII interactions reduce experimental thrombosis and inflammation. FXI inhibitors are antithrombotic in patients, and FXI and FXII deficiencies are atheroprotective in apolipoprotein E-deficient mice. Gill D, et al. Genetically determined FXI (factor XI) levels and risk of stroke. Stroke 2018; 49(11):2761-3; Salomon et al. Reduced incidence of ischemic stroke in patients with severe factor XI deficiency. Blood 2008; 111(8):4113-7. Rohmann et al. Coagulation factor XII, XI, and VIII activity levels and secondary events after first ischemic stroke. J Thromb Haemost 2020; 18(12):3316-24.

An analysis of genetically determined FXI has shown the highest levels of FXI are associated with a heightened risk of ischemic stroke. (Gill et al., Genetically Determined FXI (Factor XI) Levels and Risk of Stroke. Stroke. 2018; 49(11):2761-2763). It has been shown that patients with congenital FXI deficiency (hemophilia C) have lower risk of stroke and venous thromboembolism (Gailani et al., Factor XI as a therapeutic target. Arterioscler Thromb Vasc Biol. 2016; 36(7):1316-1322; Peyvandi, et al; European Network of Rare Bleeding Disorders Group. Coagulation factor activity and clinical bleeding severity in rare bleeding disorders: results from the European Network of Rare Bleeding Disorders. J Thromb Haemost. 2012 April; 10(4):615-621). Studies of deep venous thrombosis prophylaxis have demonstrated a reduction in venography-defined thrombosis and favorable bleeding profiles with reduction in FXI levels or inhibition of FXa. (Buller et al. Factor XI antisense oligonucleotide for prevention of venous thrombosis. N Engl J Med 2015; 372(3):232-40; Weitz et al. Effect of osocimab in preventing venous thromboembolism among patients undergoing knee arthroplasty: the FOXTROT randomized clinical trial. JAMA 2020; 323(2):130-9; Weitz et al. Milvexian for the prevention of venous thromboembolism. N Engl J Med 2021; 385(23):2161-72.)

While Factor Xa inhibitors block the tissue factor-activated coagulation process required for hemostasis as well as the intrinsic pathway, Factor XIa inhibition blocks thrombus formation through the intrinsic pathway (or the ‘contact’ system) which may be dispensable for normal hemostasis, and through an amplification pathway of thrombin generation which appears to contribute to pathological thrombus formation. Thus, FXIa inhibition has the potential for an improved benefit to risk profile than inhibitors of Factor Xa or thrombin to reduce thrombin generation thereby preventing vascular occlusion and embolism with minimal impairment of hemostasis in patients with atherosclerotic CAD, including those after an ACS event. The risk of recurrent ACS events is highest soon after the index event and additional events continue to accumulate over time due to the progressive nature of the underlying atherosclerotic disease process. Given the high rate of recurrent events post-ACS, new targets capable of preventing pathologic thrombosis, yet preserving normal hemostasis, are needed.

There are a number of elements distinguishing milvexian from other oral Factor Xla inhibitors (eg., asundexian). For example, a population pharmacokinetics model was developed to characterize milvexian pharmacokinetics using pooled milvexian plasma concentration data from healthy adult volunteers from 6 phase 1 clinical studies.

The analysis of PK data from the AXIOMATIC-SSP trial using the proprietary population PK model found that the exposure-response relationship of milvexian in patients, administration of the pharmaceutical composition comprising 25 mg of milvexian twice daily to the human patient results in milvexian plasma concentrations reaching steady-state in about 3 days. In some embodiments of any of the above aspects, a twice daily oral administration of 25 mg of milvexian to a human patient provides a steady state plasma concentration profile in about 3 days. In some embodiments of any of the above aspects, the steady state plasma concentration profile is characterized by (i) a steady state Cₘₐₓ ranging from about 217 ng/mL to about 475 ng/mL, (ii) a steady state mean (std) Cₘₐₓ of 346 (129) ng/mL, (iii) a steady state AUC₀₋₂₄ ranging from about 4350 ng*h/mL to about 10230 ng*h/mL, and (iv) a steady state mean (std) AUC₀₋₂₄ of 7290 (2940) ng*h/mL. Further, the 25 mg BID dose regimen of milvexian used for Phase 3 trial on ACS as described in Example 1 was selected based on the data from the Phase 1 and Phase 2 studies and the analysis results for the exposure-response relationship of milvexian in patients using this proprietary population pharmacokinetic model.

As used herein, the term “acute coronary syndrome” (ACS) refers to a spectrum of coronary artery diseases resulting from atherothrombosis within the epicardial coronary arteries, typically triggered by plaques rapture or erosion. Atherothrombosis is the central pathological process driving most ACS. For example, coronary plaque rupture resulting in thrombosis formation occluding a coronary artery is the underlie pathophysiology for STEMI. ACS can be divided into subgroups of ST-segment elevation myocardial infarction (STEMI), non-ST-segment elevation myocardial infarction (NSTEMI), and unstable angina. Where associated with myocardial damages, this indicates type 1 myocardial infarction (MI). If there is no myocardial injury but the clinical and electrocardiogram (ECG) findings indicate an unpredictable pattern of chest pain, this is termed unstable angina (UA). ACS can be further defined by the presence or absence of ST-segment elevation on the electrocardiogram. In the appropriate clinical context, presence of significant, territorial ST elevation typically signifies ST-elevation myocardial infarction (STEMI) secondary to acute total occlusion of a major epicardial coronary artery, resulting, if left without treatment, in a transmural infarction. ACS without ST elevation (termed non-ST-elevation acute coronary syndrome (NSTE-ACS) is usually associated with transient or partial coronary occlusion, and more often results in subendocardial MI, non-ST-elevation myocardial infarction with less myocardial damages, or unstable angina with no detectable myocardial damage.

As used herein, the term “ST segment” on an electrocardiogram (ECG) encompasses an electrically neutral area of the complex between ventricular depolarization (QRS complex) and repolarization (T wave). In clinical terms, the ST segment represents the period in which the myocardium maintains contraction to expel blood from the ventricles. The ST segment represents an important feature of the ECG complex. It can show characteristic features that can help differentiate normal vs ischemic conditions (Kashou et al., ST Segment. [Updated 2023 Aug. 14]. In: StatPearls [Internet]. Treasure Island (FL): StatPearls Publishing; 2024 Jan-. available from: www.ncbi.nlm.nih.gov/books/NBK459364/).

As used herein, the term “ST-segment elevation” refers to the ST segment becoming displaced above baseline (ST elevation) when the J point is displaced above baseline. ST-elevation is recognized as a sign of an occlusive thrombus in an acute setting. Early repolarization, LVH, ventricular aneurysm, left bundle branch block, and other conduction defects have been shown to be more common causes of ST elevation than acute MIs. It is also important to evaluate the waveform of the ST segment. Displacement of the J point can be further characterized as horizontal, upsloping, or downsloping; the latter two can be rapid or slow. For example, ST elevation in leads V and V can be described as rapidly downsloping, and in such cases, it is typically benign. These further characterizations of the ST segment can help differentiate between normal and ischemic conditions. For instance, ST elevation with an upsloping ST segment is generally considered normal, while ST elevation with a horizontal ST segment is more characteristic of myocardial ischemia (See Kashou et al. supra).

As used herein, the term “cerebrovascular disorders” refers to the neurologic problems resulting from the disruption of the blood flow to the brain that leads to damage or death of brain cells from the lack of oxygen. In some embodiments, the cerebrovascular disorders include transient ischemic attack. In some embodiments, the cerebrovascular disorders include stroke. How a stroke affects the body depends on precisely where in the brain the blood supply was cut off. In some embodiments, cerebrovascular disorders are vascular event often grouped in composites in various combinations, including ACM; CV death; MI; UA; “all stroke” or “any stroke” (ischemic, hemorrhagic, or unknown cause); ischemic stroke; ALI; major vascular (non-traumatic) limb amputation; symptomatic VTE (PE, DVT); ischemia-driven coronary revascularization; stent thrombosis; hospitalization for any reason for arterial or venous thrombotic event or neither.

In some embodiments, the term “stroke” refers to an acute episode of neurologic dysfunction that caused the death of brain tissue (cerebral infarction) resulting from lack of blood flow and insufficient oxygen to the brain. A stroke can either be ischemic or hemorrhagic. In an ischemic stroke, the blood supply to part of the brain is cut off because a blood clot has blocked a blood vessel due to either a thrombus formed locally in an abnormal artery (e.g., atherosclerosis) or an embolus that formed upstream and traveled through the bloodstream. In a hemorrhagic stroke, a blood vessel bursts, preventing normal blood flow and allowing blood to leak into an area of the brain and destroy it. Most strokes begin suddenly, develop rapidly and cause brain damage within minutes (complete stroke). Less commonly, strokes may continue to worsen for several hours to days as a steadily enlarging area of brain tissue dies (stroke in evolution). Ischemic stroke may be lacunar or non-lacunar in nature. Ischemic stroke may be cardioembolic ischemic stroke if the ischemic stroke is attributable to arterial occlusion from embolus that presumably arose from the blood clots formed in the heart or on one of its valves. In some embodiments, the ischemic stroke is non-cardioembolic ischemic stroke. Ischemic stroke was defined based on the 2013 guidelines (Sacco et al., An updated definition of stroke for the 21st century Stroke, 44 (2013), pp. 2064-2089).

The severity of stroke is measured using the National Institutes of Health Stroke Scale (NIHSS) scores in clinical trials (Kamel et al., Validation of the International Classification of Diseases, Tenth Revision Code for the National Institutes of Health Stroke Scale Score. Circ Cardiovasc Qual Outcomes, 2023, vol. 16, e009215). Stroke severity is categorized as follows: 1-4 for minor stroke, 5-15 for moderate stroke, 16-20 for moderate to severe stroke, and 21-42 for severe stroke.

In some embodiments, ischemic stroke refers to a neurological deficit attributable to a non-lacunar, acute brain infarction detected by neuroimaging (CT or MRI) and relevant to the clinical symptoms.

In some embodiments, “stroke” refers to any type of stroke (ischemic, hemorrhagic, or unknown cause).

In some embodiments, the ischemic stroke is further characterized by a National Institutes of Health Stroke Score (NIHSS)≤7.

In other embodiments, the ischemic stroke is further characterized by a National Institutes of Health Stroke Score (NIHSS) of 8-15.

In other embodiments, the ischemic stroke is further characterized by a National Institutes of Health Stroke Score (NIHSS)≤15.

In other embodiments, an ischemic stroke is further characterized by evidence of relevant intracranial or cervical arterial atherosclerotic plaque, ulceration or thrombus in a feeding artery documented by imaging (either Doppler ultrasound or CTA or MRA or catheter angiography).

In still other embodiments, an ischemic stroke is further characterized by a Modified Rankin Score. For example, in some embodiments an ischemic stroke is further characterized by a Modified Rankin Score (mRS) of ≤3, of ≤4, of ≤5, or of ≤6.

In some embodiments, MI is defined in accordance with the 4th Universal Definition of MI, excluding type 2 MI (Thygesen et al., Fourth universal definition of myocardial infarction (2018) Eur. Heart J., 40 (2019), pp. 237-269).

In some embodiments, Cardiovascular death is coded when the primary cause of death was MI, stroke, thromboembolism of any other vascular bed, heart failure, primary arrhythmia or a cardiovascular procedure.

As used herein, the term ‘activated partial thromboplastin time (aPTT)” refers to a measure of the intrinsic and final common pathways of the coagulation cascade. It represents the time, in seconds, for plasma to clot after addition of phospholipid, an intrinsic pathway activator, and calcium. The name ‘Activated Partial Thromboplastin Time’ comes from the original form of the test in which only the phospholipid concentration of the test was controlled (as opposed to the phospholipid and the surface activator concentrations) and the name ‘partial thromboplastin’ was applied at the time to phospholipid preparations that accelerated clotting but did not correct the prolonged clotting times of hemophilic plasma. The term ‘partial’ means phospholipid is present but no tissue factor. The normal and reference ranges vary depending on reagent and instrument combinations, particularly with the phospholipid composition.

In some embodiments, aPTT is measured as follows: Plasma samples are incubated with Actin FS aPTT assay reagent containing a standard amount of phospholipid and contact activator (ellagic acid) which activates the intrinsic coagulation pathway. After incubating for 3 minutes, calcium chloride is added to initiate coagulation and formation of a fibrin clot is measured optically. The time to clot formation (measured in seconds) is reported as the Activated Partial Thromboplastin Time (aPTT). In other embodiments, formation of the fibrin clot is measured by mechanical methods (viscosity).

Administration of multiple oral doses of milvexian to healthy human subjects resulted in a dose- and concentration-dependent prolongation of aPTT. The maximal mean change of aPTT from baseline was approximately a 1.1- to 4.1-fold increase after QD doses of 5 to 500 mg for 2 weeks and a 3.4-fold increase after BID dose of 200 mg for 2 weeks.

As used herein, a prothrombin time (PT) test measures how long it takes for a clot to form in a blood sample and the testing result from PT assay is called prothrombin time (unit second). Prothrombin time was not impacted by the administration of a single or multiple doses of milvexian to human subjects, with a maximal mean percent change from baseline of approximately 5%.

As used herein, “Factor XI Clotting Activity” is determined utilizing an aPTT-based 1-stage clotting time assay. Serial dilutions of normal pooled plasma are mixed with FXI-depleted plasma and the clotting times are measured according to standard aPTT protocol, to establish a reference range. Subject test plasma is treated in the same way and compared with the reference plasma.

In some embodiments, the Factor XI Clotting Activity is measured as follows: Factor XI (FXI) activity is measured using a modification of the activated partial thromboplastin time (aPTT) using Actin FS (Siemens Healthcare) on the Siemens BCS®XP analyzer. A 6-point calibration curve (~5-150%) is prepared using a secondary calibrator (Standard Human Plasma, Siemens Healthcare Diagnostics Inc.) with a known concentration of human FXI assigned by the manufacturer. The reference standard, at approximately 100%, is diluted by the BCS®XP analyzer in saline to generate pre-selected calibration levels of FXI. The calibration curve is plotted with FXI activity in percent (%) on the x-axis and clotting time in seconds on the y-axis. A log/lin regression curve fit is used. The samples to be tested are mixed with FXI deficient plasma (containing less than 1% FXI and at least 75% of all the other factors) to normalize all other factors. APTT reagent (Actin FS) is added and the mixture is incubated. Following incubation, calcium chloride is added to the mixture and the time to clot formation (measured optically) is compared to the time on the calibration curve. Samples are tested at the base dilution (1:10) prepared by the BCS®XP in saline. In other embodiments, formation of the fibrin clot is measured by mechanical methods (viscosity).

As used herein, “Thrombin Generation Assay (TGA)” is a global coagulation assay that evaluates the thrombogenic capacity of a plasma sample and has been proposed that it may better reflect prothrombotic or hemorrhagic states than conventional clotting assays. In the traditional TGA, coagulation of citrated plasma is initiated through the extrinsic pathway by adding tissue factor, phospholipids, and calcium. For this study, in the in-vitro thrombin generation assay (TGA) using human platelet-rich plasma, and a kaolin slurry was used instead to initiate coagulation through the contact activation pathway. Thrombin generation is continuously monitored through the product released by cleavage of a thrombin-specific fluorogenic substrate. The generated thrombogram is used to determine several relevant parameters, including endogenous thrombin potential, defined as the area under the thrombin concentration vs. time curve. In some embodiments, administration of a single dose or multiple oral doses of milvexian to a human subject results in inhibition of thrombin generation via the intrinsic pathway, but only minimal inhibition of thrombin generation when initiated by the extrinsic pathway.

As used herein, “preventing” refers to reducing the risk of occurrence. In some embodiments, preventing encompasses eliminating the risk of occurrence (i.e., reducing the risk of occurrence to zero). As such, “prevention” covers the preventive treatment aimed at reducing the probability of the occurrence of a clinical disease-state. In some embodiments, the treatment regimen as described herein is given to patients at risk of developing thromboembolic disease to prevent formation of an occlusive thrombus (primary prevention). In some embodiments, the treatment regimen as described herein is given to patients for secondary prevention, following an initial thrombotic episode, for example, secondary prevention of cardiovascular events in patients with a history of acute myocardial infarction or acute coronary syndrome. In a clinical setting, a combination of aspirin and clopidogrel (or other thienopyridines) may be used to prevent a second thrombotic event.

In some embodiments, “preventing” is synonymous with “reducing the risk” or “reducing the incidence rate” of an adverse atherosclerotic event (e.g., a MACE) occurring. Reducing the risk or reducing the incidence rate means that there is numerical and/or a statistically-significant reduction or lowering in occurrence of the adverse atherosclerotic event by at least 1% or greater. Preferably, this reduction is by 2% or greater, 3% or greater, 4% or greater, 5% or greater, 6% or greater, 7% or greater, 10% or greater, 20% or greater, 26% or greater, 34% or greater, 50% or greater, 64% or greater and 74% or greater. These reductions include confidence intervals equals or greater than 50%, equals or greater than 75%, equals or greater than 80%, equals or greater than 90%, equals or greater than 95%, equals or greater than 98% and equals or greater than 99%. Confidence intervals of equals or greater than 95% are preferred.

Within the scope of this disclosure, “prophylaxis” is the protective treatment of a disease state to reduce and/or minimize the risk and/or reduction in the risk of recurrence of a disease state by administering to a patient a therapeutically effective amount of milvexian or a pharmaceutically acceptable salt, or a solvate thereof. Patients may be selected for prophylaxis therapy based on factors that are known to increase risk of suffering a clinical disease state compared to the general population. For prophylaxis treatment, conditions of the clinical disease state may or may not be presented yet. “Prophylaxis” treatment can be divided into (a) primary prophylaxis and (b) secondary prophylaxis. Primary prophylaxis is defined as treatment to reduce or minimize the risk of a disease state in a patient that has not yet presented with a clinical disease state, whereas secondary prophylaxis is defined as minimizing or reducing the risk of a recurrence or second occurrence of the same or similar clinical disease state.

As used herein, the term “treating” refers to ameliorating the signs or symptoms of a disease or condition in a patient and/or preventing a disease or condition in a patient. As used herein, unless otherwise noted, the terms “treating”, “treatment” and the like, shall include the management and care of a patient for the purpose of combating a disease, condition, or disorder and includes the administration of milvexian to prevent the onset of the symptoms or complications, alleviate the symptoms or complications, or eliminate the disease, condition, or disorder. As such, “treating” or “treatment” cover the treatment of a disease-state in a human, and include: (a) inhibiting the disease-state, i.e., arresting its development; and/or (b) relieving the disease-state, i.e., causing regression of the disease state.

As used herein, a “risk factor” is a demographic factor that influences the underlying risk of an event independently of any drug treatment.

As used herein, the term “thrombotic event” refers to adverse cerebrovascular and/or cardiovascular events occurring in patients with acute coronary syndrome. In some embodiments, the thrombotic event comprises major adverse cardiovascular event (MACE), atherothrombosis, ischemic stroke, myocardial infarction, cardiovascular death, arrhythmogenic cardiomyopathy, Major Adverse Vascular Events (MAVE), major adverse limb events (MALE), All-Cause Mortality (ACM), symptomatic VTE, or combinations thereof. In some embodiments, the thrombotic event comprises major adverse cardiovascular event. In some embodiments, the thrombotic event comprises ischemic stroke. In some embodiments, the thrombotic event comprises a major adverse vascular event. In some embodiments, the thrombotic event comprises arrhythmogenic cardiomyopathy. In some embodiments, the thrombotic event comprises a major adverse limb events (MALE).

As used herein, the term “atherosclerotic event” refers to a major adverse cardiovascular events (MACE), a major adverse vascular event (MAVE), arrhythmogenic cardiomyopathy, a major adverse limb events (MALE), or a combination thereof.

As used herein, the term “major adverse cardiovascular event” or “MACE” refers to cardiovascular death, nonfatal myocardial infarction, ischemic stroke, or combinations thereof.

As used herein, the term “major adverse vascular event” or “MAVE” refers to cardiovascular death, nonfatal myocardial infarction, ischemic stroke, MALE, symptomatic VTE, or combinations thereof.

As used herein, the term “major adverse limb event” or “MALE” refers to acute limb ischemia (ALI), major nontraumatic vascular amputation, or combinations thereof.

As used herein, the term “symptomatic VTE” refers to pulmonary embolism, deep vein thrombosis, or combinations thereof.

As used herein, “pharmaceutically acceptable salt” refers to derivatives wherein a compound is modified by making an acid or a basic salts thereof Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic. The pharmaceutically acceptable salts of milvexian can be synthesized using conventional chemical methods. Generally, such salts can be prepared by reacting milvexian with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, Pa. (1990), the disclosure of which is hereby incorporated by reference.

As used herein, the term “standard of care” refers to a treatment process that is generally accepted by medical experts as a proper treatment for a certain type of disease (e.g., acute coronary syndrome) and that is widely used by healthcare professionals. In some embodiments, the standard of care comprises antiplatelet therapy for use in patients diagnosed with acute coronary syndrome. In some embodiments, the antiplatelet therapy is selected from a single antiplatelet therapy (SAPT), a dual antiplatelet therapy (DAPT), or combination thereof. In some embodiments, SAPT is selected from aspirin and a P2Y12 inhibitor. In some embodiments, SAPT comprises low-dose aspirin (≤100 mg per day), or a P2Y12 inhibitor. In some embodiments, SAPT comprises aspirin. In some embodiments, SAPT comprising a P2Y12 inhibitor. In some embodiments, the P2Y12 inhibitor (alone or in combination with aspirin) is clopidogrel, ticagrelor, or prasugrel. In some embodiments, P2Y12 inhibitor may be changed or stopped. In some embodiments, the antiplatelet therapy de-escalation from DAPT to SAPT can drop either aspirin or P2Y12 inhibitor.

As used herein “safe” means that the regimen provides a net clinical benefit such that the risk reduction for an atherosclerotic event in a human outweighs the increased risk of an adverse event, for example, serious bleeding, as compared to antiplatelet therapy alone, as determined by a regulatory agency.

As used herein “effective” means that the regimen provides risk reduction and treatment for an atherosclerotic event as compared to antiplatelet therapy alone.

Absolute risk reduction (ARR) is the percentage of patients in the control group (i.e., the group not receiving the regimen) who have a bad outcome minus the percentage of patients in the treatment group (i.e., the group receiving the regimen) who have a bad outcome. For example, if the incidence rate in the control group is 20% (i.e., if 20% of the patients in the control group experience a bad outcome), and the incidence rate in the treatment group is 12% (i.e. 12% of the patients in experience a bad outcome), then the absolute risk reduction (ARR) is 8% (i.e., 20%-12%).

In some embodiments, the “Bleeding Academic Research Consortium (BARC) criteria are as set forth in Roxana Mehran, et al. Standardized Bleeding Definitions for Cardiovascular Clinical Trials, Circulation. 2011; 123:2736-2747, which is incorporated by reference herein.

In other embodiments, the “Bleeding Academic Research Consortium (BARC) criteria are as set forth in Pascal Vranckx, et al. Validation of BARC Bleeding Criteria in Patients With Acute Coronary Syndromes J Am Coll Cardiol 2016; 67:2135-44, which is incorporated by reference herein.

In some embodiments, the GUSTO criteria are as set forth in “An international randomized trial comparing four thrombolytic strategies for acute myocardial infarction.” The GUSTO investigators. N Engl J Med 1993; 329:673-82, which is incorporated by reference herein.

In some embodiments, the GUSTO criteria are as set forth in Pascal Vranckx, et al. Validation of BARC Bleeding Criteria in Patients With Acute Coronary Syndromes J Am Coll Cardiol 2016; 67:2135-44, which is incorporated by reference herein.

In some embodiments, the TIMI criteria are as set forth in Rao AK, Pratt C, Berke A, et al. Thrombolysis in Myocardial Infarction (TIMI) Trial—phase I: hemorrhagic manifestations and changes in plasma fibrinogen and the fibrinolytic system in patients treated with recombinant tissue plasminogen activator and streptokinase. J Am Coll Cardiol 1988; 11: 1-11. which is incorporated by reference herein.

In some embodiments, the TIMI criteria are as set forth in Pascal Vranckx, et al. Validation of BARC Bleeding Criteria in Patients With Acute Coronary Syndromes J Am Coll Cardiol 2016; 67:2135-44, which is incorporated by reference herein.

In some embodiments, the “Bleeding Academic Research Consortium (BARC) Type 3 criteria” are: a. Overt bleeding plus hemoglobin drop of 3 to <5 g/dL (provided hemoglobin drop is related to bleed); transfusion with overt bleeding; b. Overt bleeding plus hemoglobin drop ≥5 g/dL (provided hemoglobin drop is related to bleed); cardiac tamponade; bleeding requiring surgical intervention for control; bleeding requiring IV vasoactive agents; or c. Intracranial hemorrhage confirmed by autopsy, imaging, or lumbar puncture; intraocular bleed compromising vision. See, e.g., Roxana Mehran, et al. Standardized Bleeding Definitions for Cardiovascular Clinical Trials, Circulation. 2011; 123:2736-2747.

In some embodiments, the “Bleeding Academic Research Consortium (BARC) Type 5 criteria” are: a. Probable fatal bleeding; or b. Definite fatal bleeding (overt or autopsy or imaging confirmation). See, e.g., Roxana Mehran, et al. Standardized Bleeding Definitions for Cardiovascular Clinical Trials, Circulation. 2011; 123:2736-2747.

In some embodiments, the “Bleeding Academic Research Consortium (BARC) Type 2 criteria” are: any clinically overt sign of hemorrhage that “is actionable” and requires diagnostic studies, hospitalization, or treatment by a health care professional. See, e.g., Roxana Mehran, et al. Standardized Bleeding Definitions for Cardiovascular Clinical Trials, Circulation. 2011; 123:2736-2747.

In some embodiments, the “ISTH criteria (major bleeding or clinically-relevant non-major bleeding (CRNM)) criteria” are as follows:

ISTH major bleeding in non-surgical patients is defined as having a symptomatic presentation and:
i. Fatal bleeding, and/or
ii. Bleeding in a critical area or organ, such as intracranial, intraspinal, intraocular, retroperitoneal, intra-articular or pericardial, or intramuscular with compartment syndrome, and/or
iii. Bleeding causing a fall in hemoglobin level of 20 g L⁻¹ (1.24 mmol L⁻¹) or more, or leading to transfusion of two or more units of whole blood or red cell

ISTH CRNM bleeding is any sign or symptom of hemorrhage (e.g., more bleeding than would be expected for a clinical circumstance, including bleeding found by imaging alone) that does not fit the criteria for the ISTH definition of major bleeding but does meet at least one of the following criteria:
i. requiring medical intervention by a healthcare professional
ii. leading to hospitalization or increased level of care
iii. prompting a face to face (i.e., not just a telephone or electronic communication) evaluation

It will be understood that the use of terms of approximation, such as “approximately” or “about,” when referring to, for example, amounts, durations of time, extents of effects, and the like, may be a number that is within 5% (greater or less than), or within 7.5% (greater or less than), or within 10% (greater or less than), or within 12.5% (greater or less than), or within 15% (greater or less than), or within 17.5% (greater or less than), or within 20% (greater or less than) of the specified number.

In some aspects, the disclosure is directed to methods for preventing adverse cerebrovascular events or adverse cardiovascular events in a human patient with acute coronary syndrome (ACS), wherein the method comprises measuring the patient's baseline FXI clotting activity, and then administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combination thereof, wherein the pharmaceutical composition is administered twice daily.

In some embodiments, the methods of the disclosure are directed to primary prevention of adverse cerebrovascular events or adverse cardiovascular events.

In some embodiments, the methods of the disclosure are directed to secondary prevention of adverse cerebrovascular events or adverse cardiovascular events.

In some embodiments of the disclosed methods, the adverse cerebrovascular event or adverse cardiovascular events comprise one or more of stroke, heart attack, or death.

In some embodiments of the disclosed methods, the adverse cerebrovascular event or adverse cardiovascular event occurs in an organ selected from heart, brain, limb, blood supply system, or vessel.

In some embodiments of the disclosed methods, the adverse cerebrovascular event or adverse cardiovascular event comprises one or more major adverse cardiovascular event (MACE) selected from the group consisting of cardiovascular death, nonfatal myocardial infarction, ischemic stroke, and combinations thereof.

In other embodiments of the disclosed methods, the adverse cerebrovascular event or adverse cardiovascular event comprises one or more major adverse vascular events (MAVE) selected from the group consisting of MACE, major adverse limb events (MALE) including one or more selected from acute limb ischemia, chronic limb ischemia, or major amputation; symptomatic venous thromboembolic events, and combinations thereof.

In some embodiments of the disclosed methods, the adverse cerebrovascular event or adverse cardiovascular event is selected from the group consisting of arrhythmogenic cardiomyopathy (ACM), nonfatal myocardial infarction, ischemic stroke, and combinations thereof.

In some embodiments of the disclosed methods, the adverse cerebrovascular event or adverse cardiovascular evens comprises cardiovascular death.

In some embodiments of the disclosed methods, the adverse cerebrovascular event or adverse cardiovascular event comprises arrhythmogenic cardiomyopathy (ACM).

In some aspects, the disclosure is directed to methods for treating or preventing adverse cerebrovascular event or adverse cardiovascular event in a human patient with acute coronary syndrome.

In some embodiments, the acute coronary syndrome is ST-segment elevation myocardial infarction (STEMI), non-ST-segment elevation acute coronary syndrome (NSTEM-ACS), non-ST-segment elevation myocardial infarction (NSTEMI), or unstable angina. In some embodiments, the acute coronary syndrome comprises ST-segment elevation myocardial infarction. In some embodiments, the acute coronary syndrome comprises non-ST-segment elevation acute coronary syndrome. In some embodiments, the acute coronary syndrome comprises non-ST-segment elevation myocardial infarction. In some embodiments, the acute coronary syndrome comprises unstable angina.

In some embodiments, the human patient has NSTEM-ACS without atrial fibrillation. In some embodiments, the antiplatelet therapy comprises aspirin 150 mg-300 mg as a loading dose followed by aspirin monotherapy at 75 mg-100 mg once daily as maintaining dose. In some embodiments, the maintaining dose of aspirin may be administered for a period ranging from 3 months to 3 years. In some embodiments, the maintaining dose of aspirin may be administered for a period ranging from 3 months to 1 year. In some embodiments, the maintaining dose of aspirin may be administered for a period of 1 year. In some embodiments, the antiplatelet therapy comprises aspirin and a P2Y12 inhibitor.

In some embodiments, the human patient has NSTEM-ACS and atrial fibrillation.

In some embodiments, the human patient is a female. In other embodiments, the human patient is a male.

In some embodiments, the human patient is at least 40 years old.

In other embodiments, the human patient is at least 50 years old.

In other embodiments, the human patient is at least 60 years old.

In other embodiments, the human patient is at least 70 years old.

In some aspects, the methods of the disclosure comprise administering to the human patient a regimen comprising: (i) a pharmaceutical composition comprising 25 mg of milvexian (or pharmaceutically acceptable salt or solvate thereof), or comprising 12.5 mg to 200 mg of milvexian (or pharmaceutically acceptable salt or solvate thereof); and (ii) an antiplatelet therapy selected from the group consisting of aspirin, a P2Y12 inhibitor, and combination thereof.

In some aspects, the methods of the disclosure comprise administering to the human patient a regimen comprising: (i) a pharmaceutical composition comprising 25 mg of milvexian (or pharmaceutically acceptable salt or solvate thereof) twice daily; and (ii) an antiplatelet therapy selected from the group consisting of aspirin, a P2Y12 inhibitor, and combination thereof.

In some embodiments, the pharmaceutical composition comprises milvexian.

In some embodiments, the pharmaceutical composition comprises a solvate of milvexian.

In other embodiments, the pharmaceutical composition a pharmaceutically acceptable salt of milvexian.

In embodiments of the disclosed methods in which the pharmaceutical composition comprises a solvate or a pharmaceutically acceptable salt of milvexian, then the amount specified is on a milvexian basis. That is, an amount of the solvate or the pharmaceutically acceptable salt in the pharmaceutical composition contains the specified amount of milvexian. For example, a pharmaceutical composition comprising 25 mg of milvexian (or pharmaceutically acceptable salt or solvate thereof) refers to a pharmaceutical composition comprising either 25 mg of milvexian, or an amount of a pharmaceutically acceptable salt or solvate of milvexian equivalent to 25 mg of milvexian.

In some embodiments of the disclosed methods, the regimen comprises a pharmaceutical composition comprising 12.5 mg of milvexian (or pharmaceutically acceptable salt or solvate thereof) administered twice daily.

In some embodiments of the disclosed methods, the regimen comprises a pharmaceutical composition comprising 25 mg of milvexian (or pharmaceutically acceptable salt or solvate thereof) administered twice daily.

In some embodiments of the disclosed methods, the regimen comprises a pharmaceutical composition comprising 50 mg of milvexian (or pharmaceutically acceptable salt or solvate thereof) administered twice daily.

In some embodiments of the disclosed methods, the regimen comprises a pharmaceutical composition comprising 100 mg of milvexian (or pharmaceutically acceptable salt or solvate thereof) administered twice daily.

In some embodiments of the disclosed methods, the regimen comprises a pharmaceutical composition comprising 12.5 mg to 200 mg of milvexian (or pharmaceutically acceptable salt or solvate thereof) administered twice daily.

In some embodiments of the methods of the disclosure in which the human patient is administered the composition comprising milvexian (or a pharmaceutically acceptable salt or solvate thereof, on a milvexian basis) twice daily, one of the two administrations is made in the morning, and the other of the two administrations is made in the evening, and the administrations are made at approximately the same time each day.

In some aspects of the methods of the disclosure, the pharmaceutical composition comprising milvexian (or a pharmaceutically acceptable salt or solvate thereof, on a milvexian basis) is administered twice daily for at least 12 weeks.

In some aspects of the methods of the disclosure, the pharmaceutical composition comprising milvexian (or a pharmaceutically acceptable salt or solvate thereof, on a milvexian basis) is administered twice daily for at least 13 weeks.

In some embodiments, the pharmaceutical composition comprising milvexian (or a pharmaceutically acceptable salt or solvate thereof, on a milvexian basis) is administered twice daily for 13 weeks.

In other embodiments, the pharmaceutical composition comprising milvexian (or a pharmaceutically acceptable salt or solvate thereof, on a milvexian basis) is administered twice daily for more than 13 weeks.

In other embodiments, the pharmaceutical composition comprising milvexian (or a pharmaceutically acceptable salt or solvate thereof, on a milvexian basis) is administered twice daily for at least 26 weeks, at least 52 weeks, at least 78 weeks, at least 104 weeks, at least 130 weeks, at least 156 weeks, at least 182 weeks, or at least 208 weeks.

In some embodiments, the pharmaceutical composition comprising milvexian (or a pharmaceutically acceptable salt or solvate thereof, on a milvexian basis) is administered twice daily chronically, i.e., indefinitely.

In some aspects, the regimen used in the disclosed methods comprises administering an antiplatelet therapy selected from the group consisting of aspirin, a P2Y12 inhibitor, and combination thereof.

In some embodiments of the disclosed methods, the antiplatelet therapy comprises single antiplatelet therapy (SAPT).

In some embodiments of the disclosed methods, the antiplatelet therapy comprises dual antiplatelet therapy (DAPT).

In some embodiments, the antiplatelet therapy comprises dual antiplatelet therapy (DAPT) for 21 days, followed by single antiplatelet therapy (SAPT) thereafter.

In some embodiments of the disclosed methods, the regimen comprises aspirin and clopidogrel combination therapy from day 1 to day 21, followed by aspirin monotherapy for at least 90 days.

In some embodiments of the disclosed methods, the regimen comprises aspirin and clopidogrel combination therapy from day 1 to day 21, followed by aspirin monotherapy from day 22 to day 90 day.

In some embodiments of the disclosed methods, the regimen comprises DAPT for >90 days (with or without de-escalation to SAPT).

In some embodiments of the disclosed methods, the regimen comprises DAPT for >90 days (with de-escalation to SAPT).

In other embodiments of the disclosed methods, the regimen comprises DAPT for >90 days (without de-escalation to SAPT).

In some embodiments of the disclosed methods, the regimen comprises DAPT for ≤90 days with de-escalation to SAPT.

In some embodiments of the disclosed methods, the regimen comprises SAPT.

In some embodiments of the disclosed methods, the regimen comprises administering aspirin and/or clopidogrel without milvexian dose adjustment.

In some embodiments, the single antiplatelet therapy is aspirin.

In some embodiments of the single antiplatelet therapy, aspirin is administered in an amount of 50-150 mg daily, such as, for example, one of: 50 mg, 55 mg, 60 mg, 65 mg, 70 mg, 75 mg, 80 mg, 81 mg, 85 mg, 90 mg, 95 mg, 100 mg, 105 mg, 110 mg, 115 mg, 120 mg, 125 mg, 130 mg, 135 mg, 140 mg, 145 mg, or 150 mg, daily.

In some embodiments of the single antiplatelet therapy, the aspirin is administered in an amount of 75-100 mg daily, such as, for example, one of: 75 mg, 80 mg, 81 mg, 85 mg, 90 mg, 95 mg, or 100 mg, daily.

In some embodiments of the single antiplatelet therapy, the aspirin is administered in an amount of 75 mg daily.

In some embodiments of the single antiplatelet therapy, the aspirin is administered in an amount of 81 mg daily.

In some embodiments of the single antiplatelet therapy, the aspirin is administered in an amount of 100 mg daily.

In other embodiments, the single antiplatelet therapy is a P2Y12 inhibitor.

In some embodiments of the single antiplatelet therapy, the P2Y12 inhibitor is clopidogrel, ticagrelor, or prasugrel.

In some embodiments of the single antiplatelet therapy, the P2Y12 inhibitor is clopidogrel.

In some embodiments of the single antiplatelet therapy, the clopidogrel is administered in an amount of 75 mg to 600 mg daily, such as, for example, one of: 75 mg, 150 mg, 225 mg, 300 mg, 375 mg, 450 mg, 525 mg, or 600 mg daily.

In some embodiments of the single antiplatelet therapy, the P2Y12 inhibitor is ticagrelor.

In some embodiments of the single antiplatelet therapy, the P2Y12 inhibitor is prasugrel.

In some embodiments, the single antiplatelet therapy is ticlopidine.

In some embodiments, the ticlopidine is administered in amounts an amount of 250-500 mg daily, such as, for example, 150 mg, or 500 mg daily.

In other embodiments of the disclosed methods, the antiplatelet therapy comprises dual antiplatelet therapy.

In some embodiments, the dual antiplatelet therapy is aspirin and ticlopidine.

In some embodiments, the dual antiplatelet therapy is aspirin and a P2Y12 inhibitor.

In some embodiments of the dual antiplatelet therapy, the aspirin is administered in an amount of 50-150 mg daily, such as, for example, one of: 50 mg, 55 mg, 60 mg, 65 mg, 70 mg, 75 mg, 80 mg, 81 mg, 85 mg, 90 mg, 95 mg, 100 mg, 105 mg, 110 mg, 115 mg, 120 mg, 125 mg, 130 mg, 135 mg, 140 mg, 145 mg, or 150 mg, daily.

In some embodiments of the dual antiplatelet therapy, the aspirin is administered in an amount of 75-100 mg daily, such as, for example, one of 75 mg, 80 mg, 81 mg, 85 mg, 90 mg, 95 mg, or 100 mg, daily.

In some embodiments of the dual antiplatelet therapy, the aspirin is administered in an amount of 75 mg daily.

In some embodiments of the dual antiplatelet therapy, the aspirin is administered in an amount of 81 mg daily.

In some embodiments of the dual antiplatelet therapy, the aspirin is administered in an amount of 100 mg daily.

In some embodiments of the dual antiplatelet therapy, the P2Y12 inhibitor is clopidogrel, ticagrelor, or prasugrel.

In some embodiments of the dual antiplatelet therapy, the P2Y12 inhibitor is clopidogrel.

In some embodiments of the dual antiplatelet therapy, the clopidogrel is administered in an amount of 75 mg to 600 mg daily, such as, for example, one of: 75 mg, 150 mg, 225 mg, 300 mg, 375 mg, 450 mg, 525 mg, or 600 mg daily.

In some embodiments of the dual antiplatelet therapy, the clopidogrel is administered as a loading dose. The loading dose is administered once at the start of therapy.

In some embodiments, the loading dose is between 300-600 mg daily. In some embodiments, the loading dose is 300 mg. In other embodiments, the loading dose is 600 mg.

In some embodiments of the dual antiplatelet therapy, the clopidogrel is administered in an amount of 75 mg daily.

In some embodiments of the dual antiplatelet therapy, the clopidogrel is administered in an amount of 150 mg daily.

In some embodiments of the dual antiplatelet therapy, the clopidogrel is administered in an amount of 225 mg daily.

In some embodiments of the dual antiplatelet therapy, the clopidogrel is administered in an amount of 300 mg daily.

In some embodiments of the dual antiplatelet therapy, the clopidogrel is administered in an amount of 375 mg daily.

In some embodiments of the dual antiplatelet therapy, the clopidogrel is administered in an amount of 450 mg daily.

In some embodiments of the dual antiplatelet therapy, the clopidogrel is administered in an amount of 525 mg daily.

In some embodiments of the dual antiplatelet therapy, the clopidogrel is administered in an amount of 600 mg daily.

In some embodiments, the antiplatelet therapy is a dual antiplatelet therapy (DAPT) comprising aspirin 75-100 mg QD and a P2Y12 inhibitor. In some embodiments, the antiplatelet therapy is a single antiplatelet therapy (SAPT). In some embodiments, the method comprises administering an antiplatelet therapy consisting essentially of a dual antiplatelet therapy and a single antiplatelet therapy. In some embodiments, the antiplatelet therapy comprises DAPT.

In some embodiments, the DAPT comprises aspirin and prasugrel. In some embodiments, the DAPT comprises aspirin and prasugrel administered for 12 months.

In some embodiments, the DAPT comprises aspirin and clopidogrel. In some embodiments, the aspirin and clopidogrel is administered for 21 days to 12 months. In some embodiments, the DAPT comprises aspirin and clopidogrel. In some embodiments, the aspirin and clopidogrel is administered for 21 days. In some embodiments, the aspirin and clopidogrel is administered for 6 months. In some embodiments, the aspirin and clopidogrel is administered for 12 months.

In some embodiments, the DAPT comprises aspirin and ticagrelor. In some embodiments, the DAPT comprises aspirin and ticagrelor administered for 12 months. In some embodiments, the DAPT comprises aspirin 75-100 mg QD and a P2Y12 inhibitor administered first for 12 months followed by aspirin monotherapy for 6 months to 12 months.

In some embodiments, the DAPT comprises aspirin 75-100 mg QD and a P2Y12 inhibitor administered first for 21 days followed by aspirin monotherapy for 6 months.

In some embodiments, the DAPT comprises aspirin 75-100 mg QD and a P2Y12 inhibitor administered first for 21 days followed by aspirin monotherapy for 12 months.

In some embodiments, the DAPT comprises aspirin 75-100 mg QD and clopidogrel 75 mg QD administered first for 21 days followed by aspirin monotherapy for 6 months.

In some embodiments, the DAPT comprises aspirin 75-100 mg QD and ticagrelor 90 mg BID administered first for 12 months followed by aspirin 75-100 mg QD for 12 months.

In some embodiments, the DAPT comprises aspirin 75-100 mg QD and ticagrelor 90 mg BID administered first for 12 months followed by ticagrelor 90 mg BID for 23 months.

In some embodiments, the DAPT comprise aspirin 75-100 mg QD and 75 mg ticagrelor BID. In some embodiments, the aspirin 75-100 mg QD and 75 mg ticagrelor BID is administered for 12 months.

In some embodiments, the DAPT comprises aspirin 81-100 mg QD and 90 mg ticagrelor BID. In some embodiments, the aspirin 81-100 mg QD and 90 mg ticagrelor BID is administered for 3 years.

In some embodiments, the antiplatelet therapy comprises aspirin administered for 30 days to 90 days followed by clopidogrel monotherapy. In some embodiments, the antiplatelet therapy comprises aspirin administered for 60 days followed by clopidogrel monotherapy. In some embodiments, the antiplatelet therapy comprises aspirin administered for 90 days followed by clopidogrel monotherapy.

In some embodiments, the antiplatelet therapy comprises SAPT.

In some embodiments, the SAPT comprises aspirin. In some embodiments, aspirin monotherapy is administered once daily. In some embodiments, aspirin monotherapy is administered once daily for 3 months to 3 years. In some embodiments, aspirin monotherapy is administered once daily for 3 months. In some embodiments, aspirin monotherapy is administered once daily for 6 months. In some embodiments, aspirin monotherapy is administered once daily for 12 months. In some embodiments, aspirin monotherapy is administered once daily for 24 months. In some embodiments, aspirin monotherapy is administered once daily for 36 months. In some embodiments, aspirin monotherapy is administered twice daily.

In some embodiments, the SAPT comprises a P2Y12 inhibitor. In some embodiments, the P2Y12 inhibitor is selected from prasugrel, clopidogrel, selatogrel, or ticagrelor. In some embodiments, the P2Y12 inhibitor monotherapy is administered for 3 months to 6 months.

In some embodiments, the SAPT comprises clopidogrel. In some embodiments, the SAPT comprises ticagrelor. In some embodiments, the SAPT comprises ticagrelor monotherapy followed by aspirin 20 mg twice daily.

In some embodiments, the SAPT comprises aspirin 75-100 mg QD. In some embodiments, the aspirin 75-100 mg QD is administered for 12 months.

In some embodiments, the SAPT comprises ticagrelor 90 mg BID. In some embodiments, the ticagrelor 90 mg BID is administered for 3 years.

In some embodiments, the SAPT comprises clopidogrel. In some embodiments, aspirin monotherapy is administered first followed by clopidogrel monotherapy for at least additional 2 months.

In some aspects, the disclosure is directed to methods for preventing adverse cardiovascular events in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof,
wherein the pharmaceutical composition is administered twice daily; and
wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; and transient ischemic attack (TIA).

In other embodiments of such methods, the adverse cardiovascular event is one or more of CV death, MI, or ischemic stroke.

In some aspects, the disclosure is directed to methods for reducing incidence rate of the one or more of adverse thrombotic event selected from new ischemic stroke, MI, or all-cause death in a human patient diagnosed with acute coronary syndrome, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof, wherein the regimen is administered twice daily.

In some embodiments of such methods, administration of the regimen results in a relative risk is 0.85 or less relative to the placebo, such as, for example, 0.85, 0.84, 0.83, 0.82, 0.81, 0.8, 0.79, 0.78, 0.77, 0.76, 0.75, 0.74, 0.73, 0.72, 0.71, or 0.7, relative to the placebo.

In other aspects, the disclosure is directed to methods for preventing ischemic stroke in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

In some embodiments of such methods, the clinical benefit of reducing the incidence rates of the ischemic stroke by the regimen is maintained throughout a treatment period of 90 days.

In some embodiments of the disclosed methods, the patient's baseline FXI clotting activity is measured. As used herein, the term “baseline FXI clotting activity” refers to the patient's FXI clotting activity before being administered the regimen comprising a pharmaceutical composition comprising milvexian (or pharmaceutically acceptable salt or solvate thereof).

In some embodiments, Factor XI (FXI) activity is measured using a modification of the activated partial thromboplastin time (aPTT) using Actin FS (Siemens Healthcare) on the Siemens BCS®XP analyzer. A 6-point calibration curve (-5-150%) is prepared using a secondary calibrator (Standard Human Plasma, Siemens Healthcare Diagnostics Inc.) with a known concentration of human FXI assigned by the manufacturer. The reference standard, at approximately 100%, is diluted by the BCS®XP analyzer in saline to generate pre-selected calibration levels of FXI. The calibration curve is plotted with FXI activity in percent (%) on the x-axis and clotting time in seconds on the y-axis. A log/lin regression curve fit is used. The samples to be tested are mixed with FXI deficient plasma (containing less than 1% FXI and at least 75% of all the other factors) to normalize all other factors. APTT reagent (Actin FS) is added and the mixture is incubated. Following incubation, calcium chloride is added to the mixture and the time to clot formation (measured optically) is compared to the time on the calibration curve. Samples are tested at the base dilution (1:10) prepared by the BCS®XP in saline. In other embodiments, formation of the fibrin clot is measured by mechanical methods (viscosity).

In some embodiments of the methods of the disclosure, the administration of the regimen reduces the patient's FXI clotting activity by about 7% to about 70% relative to baseline.

In other aspects, in the methods of the disclosure, the administration of the regimen reduces the patient's FXI clotting activity by about 7% to about 20% relative to baseline.

In some embodiments, in the methods of the disclosure, the administration of the regimen reduces the patient's FXI clotting activity by about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, or about 68%, about 69%, or about 70%, relative to baseline.

In other embodiments, in the methods of the disclosure, the administration of the regimen reduces the patient's FXI clotting activity by about 7%, about 8%, about 9%, about 10%, about 11%, about 12%, about 13%, about 14%, about 15%, about 16%, about 17%, about 18%, about 19%, or about 20%, relative to baseline.

In other embodiments, in the methods of the disclosure, the administration of the regimen reduces the patient's FXI clotting activity by an amount shown in Table 5 in Example 2.

In some aspects of the disclosed methods, administration of the regimen results in a prolongation of activated partial thromboplastin time (aPTT) ranging from about 27% to about 64% relative to baseline.

In some embodiments of the disclosed methods, the administration of the regimen results in a prolongation of activated partial thromboplastin time (aPTT) ranging from about 27% to about 64%, such as, for example, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, or about 64%, relative to baseline. As used here “baseline” refers to the patient's aPTT prior to any administration of the regimen.

In some embodiments of the disclosed methods, the administration of the regimen results in a prolongation of activated partial thromboplastin time (aPTT) in an amount shown in Table 4 in Example 2.

In some embodiments, aPTT is determined as follows: Plasma samples are incubated with Actin FS aPTT assay reagent containing a standard amount of phospholipid and contact activator (ellagic acid) which activates the intrinsic coagulation pathway. After incubating for 3 minutes, calcium chloride is added to initiate coagulation and formation of a fibrin clot is measured optically. The time to clot formation (measured in seconds) is reported as the Activated Partial Thromboplastin Time (aPTT). In other embodiments, formation of the fibrin clot is measured by mechanical methods (viscosity).

In some aspects of the methods of the disclosure, the pharmaceutical composition comprising milvexian (or pharmaceutically acceptable salt or solvate thereof) is a solid oral pharmaceutical composition.

In some embodiments of the methods of the disclosure, the solid oral pharmaceutical composition comprises a spray-dried amorphous solid dispersion (SDP) consisting essentially of milvexian free form and a pH-dependent enterosoluble polymer.

In some embodiments of the methods of the disclosure, the SDP comprises the milvexian free form and the pH-dependent enterosoluble polymer in a weight ratio of 3:1 (milvexian:polymer).

In some embodiments of the methods of the disclosure, the pH-dependent enterosoluble polymer is cellulose acetate trimellitate (CAT), cellulose acetate phthalate (CAP), Hydroxypropyl methylcellulose phthalate (HPMCP), hydroxypropyl methylcellulose ES grade (HPMC ES), hydroxypropyl methyl cellulose acetate succinate (HPMC-AS) LF, LG, MF, MG or HF Grades such as Agoat®, Polyvinyl acetate phthalate (PVAP) such as Sureteric® and Opadry® and Shellac resins such as SSB® Aquagold, or polyvinylpyrrolidone (PVP).

In some embodiments of the methods of the disclosure, the pH-dependent enterosoluble polymer is hydroxypropyl methyl cellulose-AS MG.

In some embodiments of the methods of the disclosure, the solid oral pharmaceutical composition further comprises a binder, such as, for example, microcrystalline cellulose (MCC), silicified microcrystalline cellulose (SMCC), or a combination thereof.

In some embodiments of the methods of the disclosure, the solid oral pharmaceutical composition further comprises a filler, such as, for example, lactose monohydrate.

In some embodiments of the methods of the disclosure, the solid oral pharmaceutical composition further comprises a disintegrant.

In some embodiments of the methods of the disclosure, the solid oral pharmaceutical composition further comprises a lubricant.

In some embodiments of the methods of the disclosure, the solid oral pharmaceutical composition is a tablet.

In some embodiments of the methods of the disclosure, the solid oral pharmaceutical composition is an immediate release tablet.

In some embodiments of the methods of the disclosure, the solid oral pharmaceutical composition is a direct compression tablet.

In some embodiments of the methods of the disclosure, the solid oral pharmaceutical composition is a roller compaction tablet.

In some embodiments of the methods of the disclosure, the solid oral pharmaceutical composition is a film-coated tablet.

In some embodiments of the methods of the disclosure, the film coating comprises polyvinyl alcohol, titanium dioxide, polyethylene glycol-polyvinyl alcohol graft copolymer, and talc.

In some embodiments of the methods of the disclosure, the film coating comprises polyethylene glycol-polyvinyl alcohol graft copolymer.

In some embodiments, the film coating comprises polyvinyl alcohol, iron oxide, macrogol (PEG) polyvinyl alcohol grafted copolymer, and talc.

In some embodiments, the film coating comprises OpadryQX 321A220063 Yellow, a film coating material that comprises polyvinyl alcohol, iron oxide, macrogol (PEG) polyvinyl alcohol grafted copolymer, and talc.

In some embodiments, the solid oral pharmaceutical composition is a tablet having the composition and/or properties shown in Table A:

TABLE A
Film-coated Tablet of Milvexian at 100 mg or 25 mg Strength

| | Amount | | |
|---|---|---|---|
| | | Amount | Amount |
| | | per dose | per dose |
| Component | wt. % | (25 mg) | (100 mg) |
| SDP^{b} | 22.22 | 33.33 | 133.3 |
| Silicified Microcrystalline | 43.07 | 64.6 | 258.4 |
| cellulose | | | |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| OpadryQX 321A220063 | 3.0 | 4.5 | 18.0 |
| Yellow^{c} | | | |
| | | 154.5 | 618 |
| Tablet hardness | | Average 60N | Average 140N to |
| | | to 120 N (Ph. | 220 N (Ph. |
| | | Eur.2.9.8), | Eur.2.9.8), preferably |
| | | preferably 90N | 180N |
| Tablet friability | | <0.5% | <0.5% |
| Disintegration time in | | 13 | 18 |
| water (seconds) | | | |

^{b}SDP: spray-dried amorphous solid dispersion prepared according to the composition and method described in WO 2020210629. SDP consists essentially of milvexian free form and hypromellose acetate succinate (HPMCAS-MG) in a weight ratio of 3:1 (milvexian: HPMCAS-MG).
^{c}OpadryQX 321A220063 Yellow: a film coating material comprises polyvinyl alcohol, iron oxide, macrogol (PEG) polyvinyl alcohol grafted copolymer, and talc.

As used herein, the term “disintegration time” refers to the time required for the tablet to break into particles under a given set of conditions. In some embodiments, the disintegration time is determined using the apparatus described in Eur. Ph. (PTZ-E Pharma Test, Hainburg, Germany), in distilled water at 37° C using disks.

In some embodiments in which the solid pharmaceutical composition is a tablet, the tablet has a disintegration time in water of less than 60 seconds at 37° C

In some embodiments in which the solid pharmaceutical composition is a tablet, the tablet has a disintegration time in water of less than 20 seconds at 37° C

In other embodiments in which the solid pharmaceutical composition is a tablet, the tablet has a disintegration time in water of less than 15 seconds at 37° C

In other embodiments in which the solid pharmaceutical composition is a tablet, the tablet has a disintegration time in water of less than 10 seconds at 37° C

In some embodiments of the methods of the disclosure, the solid pharmaceutical composition is a capsule.

In some aspects of the methods of the disclosure, the human patient to whom the pharmaceutical composition comprising milvexian (or a pharmaceutically acceptable salt or solvate thereof) is administered, is unable to swallow a tablet dosage form.

In some embodiments of the disclosed methods, the solid oral pharmaceutical composition is dispersed in an aqueous medium to form an aqueous dispersion for administration.

In some embodiments of the disclosed methods, the solid oral pharmaceutical composition is a tablet which is dispersed in an aqueous medium to form an aqueous dispersion for administration.

In some embodiments of the disclosed methods, the solid oral pharmaceutical composition is a tablet which is administered orally as an aqueous dispersion by dispersing the tablet in an aqueous medium in less than 1 minute.

In those embodiments in which the solid oral pharmaceutical composition is dispersed in an aqueous medium to form an aqueous dispersion, the aqueous medium is water, saline, phosphate buffer, vegetable juice, or a fruit juice, including, for example, apple sauce.

In those embodiments in which the solid oral pharmaceutical composition is a tablet which is dispersed in an aqueous medium to form an aqueous dispersion, the aqueous medium is water, saline, phosphate buffer, vegetable juice, or a fruit juice, including, for example, apple sauce.

In some embodiments of the disclosed methods in which the solid oral pharmaceutical composition is dispersed in an aqueous medium to form an aqueous dispersion for administration, the aqueous dispersion is administered to the human patient via a nasogastric tube or spoon.

In some embodiments of the disclosed methods in which the solid oral pharmaceutical composition is a tablet which is dispersed in an aqueous medium to form an aqueous dispersion for administration, the aqueous dispersion is administered to the human patient via a nasogastric tube or spoon.

In some embodiments of the disclosed methods, oral administration of the pharmaceutical composition comprising milvexian (or a pharmaceutically acceptable salt or solvate thereof) results in a milvexian plasma half-life ranging from about 13 hours to about 16 hours. Methods of determining plasma half-life in human patients are known to those of skill in the art (and include, e.g., the method described by the protocol in the examples below).

In some embodiments of the disclosed methods, administration of the pharmaceutical composition comprising milvexian (or a pharmaceutically acceptable salt or solvate thereof) to the human patient results in milvexian plasma concentrations reaching steady-state at about 3 days to 6 days. As used herein, the term “steady-state” refers to steady-state plasma concentrations as defined by regulatory agencies such as the U.S. Food & Drug Administration (FDA) and the European Medicines Agency (EMA).

There are a number of elements distinguishing milvexian from other oral Factor Xla inhibitors (eg., asundexian). For example, a population pharmacokinetics model was developed to characterize milvexian pharmacokinetics using pooled milvexian plasma concentration data from healthy adult volunteers from 6 phase 1 clinical studies.

The analysis of PK data from the AXIOMATIC-SSP trial using the proprietary population PK model found that the exposure-response relationship of milvexian in patients, administration of the pharmaceutical composition comprising 25 mg of milvexian twice daily to the human patient results in milvexian plasma concentrations reaching steady-state in about 3 days. In some embodiments of any of the above aspects, a twice daily oral administration of 25 mg of milvexian to a human patient provides a steady state plasma concentration profile in about 3 days. In some embodiments of any of the above aspects, the steady state plasma concentration profile is characterized by (i) a steady state Cₘₐₓ ranging from about 217 ng/mL to about 475 ng/mL, (ii) a steady state mean (std) Cₘₐₓ of 346 (129) ng/mL, (iii) a steady state AUC0-24 ranging from about 4350 ng*h/mL to about 10230 ng*h/mL, and (iv) a steady state mean (std) AUC0-24 of 7290 (2940) ng*h/mL. Further, the 25 mg BID dose regimen of milvexian used for Phase 3 trial on ACS as described in Example 1 was selected based on the data from the Phase 1 and Phase 2 studies and the analysis results for the exposure-response relationship of milvexian in patients using this proprietary population pharmacokinetic model.

In some embodiments of the disclosed methods, the pharmaceutical composition comprising milvexian (or a pharmaceutically acceptable salt or solvate thereof) is administered without regard to the timing of food intake.

In some aspects of the disclosed methods, the administration does not result in a statistically significant increase in major bleeding complications. In these aspects, “statistically significant increase” refers to an increase from the patient's baseline major bleeding, i.e., the patient's major bleeding before being administered the pharmaceutical composition comprising milvexian (or a pharmaceutically acceptable salt or solvate thereof).

In some embodiments of the methods of the disclosure, the administration does not result in a statistically significant increase in major bleeding complications as assessed under ISTH criteria.

In some embodiments of the methods of the disclosure, the administration does not result in a statistically significant increase in major bleeding complications as assessed by CRNM bleeding criteria.

In some embodiments of the methods of the disclosure, the administration does not result in a statistically significant increase in major bleeding complications as assessed by ISTH CRNM bleeding criteria.

In some embodiments of the methods of the disclosure, the administration does not result in a statistically significant increase in major bleeding complications as assessed by ISTH major or CRNM bleeding criteria.

In some embodiments of the methods of the disclosure, the administration does not result in a statistically significant increase in major bleeding complications as assessed by GUSTO, BARC, or TIMI criteria.

In some embodiments of the methods of the disclosure, the administration does not result in a statistically significant increase in major bleeding complications as assessed by BARC 3c and 5 categories.

In some embodiments of the methods of the disclosure, the administration does not result in a statistically significant increase in major bleeding complications as assessed by BARC 3b, 3c, and 5 categories.

In some embodiments of the methods of the disclosure, the administration does not result in a statistically significant increase in major bleeding complications as assessed by BARC 3b category.

In some embodiments of the methods of the disclosure, the administration does not result in a statistically significant increase in major bleeding complications as assessed by BARC 3c category.

In some embodiments of the methods of the disclosure, the administration does not result in a statistically significant increase in major bleeding complications as assessed by BARC 5 category.

In some embodiments of the methods of the disclosure, the administration does not result in a statistically significant increase in GUSTO severe or life-threatening bleeding.

In some embodiments of the methods of the disclosure, the administration does not result in a statistically significant increase in non-CABG TIMI major bleeding.

In some embodiments of the methods of the disclosure, the administration does not result in a statistically significant increase in clinically relevant nonmajor bleeding.

In some embodiments of the methods of the disclosure, the administration does not result in a statistically significant increase in clinically relevant nonmajor bleeding as assessed by BARC 2, BARC 3a, or BARC 4.

In some embodiments of the methods of the disclosure, the administration does not result in a statistically significant increase in clinically relevant nonmajor bleeding as assessed by ISTH nonmajor clinically relevant bleeding.

In some embodiments of the methods of the disclosure, the administration does not result in a statistically significant increase in clinically relevant nonmajor bleeding as assessed by GUSTO moderate bleeding.

In some embodiments of the methods of the disclosure, the administration does not result in a statistically significant increase in clinically relevant nonmajor bleeding as assessed by TIMI CABG-related major bleeding.

In some embodiments of the methods of the disclosure, the administration does not result in a statistically significant increase in clinically relevant nonmajor bleeding as assessed by TIMI minor bleeding.

In some embodiments of the methods of the disclosure, the administration does not result in a statistically significant increase in clinically relevant nonmajor bleeding as assessed by TIMI bleeding requiring medical attention.

In some embodiments of the methods of the disclosure, the Relative Risk of bleeding according to the Bleeding Academic Research Consortium (BARC) Type 3 or 5 criteria by the regimen is no greater than 3, such as, for example, no greater than one of: 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

In some embodiments of the methods of the disclosure, the Relative Risk of bleeding according to the Bleeding Academic Research Consortium (BARC) Type 3 or 5 criteria by the regimen is no greater than 3, such as, for example, no greater than one of: 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

In some embodiments of the disclosed methods, the Relative Risk of serious bleeding according to the Bleeding Academic Research Consortium (BARC) Type 3 or 5 criteria is independent of the amount of milvexian administered.

In other embodiments of the disclosed methods, the human patient does not suffer serious bleeding according to the Bleeding Academic Research Consortium (BARC) Type 3 and 5 criteria.

In some embodiments of the methods of the disclosure, the Relative Risk of bleeding according to the Bleeding Academic Research Consortium (BARC) Type 2 criteria by the regimen is no greater than 2.6, such as, for example, no greater than one of: 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with standard of care.

In some embodiments of the methods of the disclosure, the Relative Risk of bleeding according to the Bleeding Academic Research Consortium (BARC) Type 2 criteria by the regimen is no greater than 2.6, such as, for example, no greater than one of: 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

In some embodiments of the disclosed methods, the Relative Risk of bleeding according to the Bleeding Academic Research Consortium (BARC) Type 2 criteria is independent of the amount of milvexian administered.

In other embodiments of the disclosed methods, the human patient does not suffer bleeding according to the Bleeding Academic Research Consortium (BARC) Type 2.

In some embodiments of the methods of the disclosure, the Relative Risk of bleeding according to the ISTH criteria (major bleeding or clinically-relevant non-major bleeding (CRNM)) by the regimen is no greater than 3, such as, for example, no greater than one of 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with standard of care.

In some embodiments of the methods of the disclosure, the Relative Risk of bleeding according to the ISTH criteria (major bleeding or clinically-relevant non-major bleeding (CRNM)) by the regimen is no greater than 3, such as, for example, no greater than one of 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, or 0.5, compared to placebo with antiplatelet therapy.

Administration of milvexian according to the disclosure is generally safe and well tolerated. For example, administration of milvexian, at a dose of 25 mg, 50 mg, or 100 mg results in no clinically significant QTc interval prolongation. For example, administration of milvexian, at a dose of 25 mg, 50 mg, or 100 mg does not result in a ΔΔQTc of 10 msec or longer. In some aspects, administration of milvexian, at a dose of 25 mg, 50 mg, or 100 mg results in a ΔΔQTc of less than 10 msec. In some aspects, administration of milvexian, at a dose of 25 mg, 50 mg, or 100 mg results in ΔΔQTc of less than 9 msec. In some aspects, administration of milvexian, at a dose of 25 mg, 50 mg, or 100 mg results in a ΔΔQTc of less than 8 msec. In some aspects, administration of milvexian, at a dose of 25 mg, 50 mg, or 100 mg results in a ΔΔQTc in QTc of less than 7 msec. In some aspects, administration of milvexian, at a dose of 25 mg, 50 mg, or 100 mg results in a ΔΔQTc of less than 6 msec. In some aspects, administration of milvexian, at a dose of 25 mg, 50 mg, or 100 mg results in a ΔΔQTc of less than 5 msec. In some aspects, administration of milvexian, at a dose of 25 mg, 50 mg, or 100 mg results in a ΔΔQTc of less than 4 msec.

Administration of milvexian, at a dose of 25 mg results in no clinically significant QTc interval prolongation. In some aspects, administration of milvexian, at a dose of 25 mg, results in a ΔΔQTc of less than 10 msec. In some aspects, administration of milvexian, at a dose of 25 mg, results in a ΔΔQTc of less than 9 msec. In some aspects, administration of milvexian, at a dose of 25 mg, results in a ΔΔQTc of less than 8 msec. In some aspects, administration of milvexian, at a dose of 25 mg, results in a ΔΔQTc of less than 7 msec. In some aspects, administration of milvexian, at a dose of 25 mg, results in a ΔΔQTc of less than 6 msec. In some aspects, administration of milvexian, at a dose of 25 mg, results in a ΔΔQTc of less than 5 msec. In some aspects, administration of milvexian, at a dose of 25 mg, results in a ΔΔQTc of less than 4 msec.

Administration of milvexian, at a dose of 50 mg results in no clinically significant QTc interval prolongation. In some aspects, administration of milvexian, at a dose of 50 mg, results in a ΔΔQTc of less than 10 msec. In some aspects, administration of milvexian, at a dose of 50 mg, results in a ΔΔQTc of less than 9 msec. In some aspects, administration of milvexian, at a dose of 50 mg, results in a ΔΔQTc of less than 8 msec. In some aspects, administration of milvexian, at a dose of 50 mg, results in a ΔΔQTc of less than 7 msec. In some aspects, administration of milvexian, at a dose of 50 mg, results in a ΔΔQTc of less than 6 msec. In some aspects, administration of milvexian, at a dose of 50 mg, results in a ΔΔQTc of less than 5 msec. In some aspects, administration of milvexian, at a dose of 50 mg, results in a ΔΔQTc of less than 4 msec.

Administration of milvexian, at a dose of 100 mg results in no clinically significant QTc interval prolongation. In some aspects, administration of milvexian, at a dose of 100 mg, results in a ΔΔQTc of less than 10 msec. In some aspects, administration of milvexian, at a dose of 100 mg, results in a ΔΔQTc of less than 9 msec. In some aspects, administration of milvexian, at a dose of 100 mg, results in a ΔΔQTc of less than 8 msec. In some aspects, administration of milvexian, at a dose of 100 mg, results in a ΔΔQTc of less than 7 msec. In some aspects, administration of milvexian, at a dose of 100 mg, results in a ΔΔQTc of less than 6 msec. In some aspects, administration of milvexian, at a dose of 100 mg, results in a ΔΔQTc of less than 5 msec. In some aspects, administration of milvexian, at a dose of 100 mg, results in a ΔΔQTc of less than 4 msec.

The disclosure is directed to a method of preventing a thrombotic event in an adult patient, after an acute coronary syndrome (ACS), comprising administering 25 mg BID milvexian in combination with antiplatelet therapy. The disclosure is directed to methods of preventing thrombotic events in adult patients, after an acute coronary syndrome (ACS), comprising administering 25 mg BID milvexian in combination with antiplatelet therapy.

### ASPECTS

It will be understood that references herein to methods of using milvexian or compositions comprising milvexian for treating or preventing the conditions of the disclosure should also be interpreted as references to: (i) the milvexian or compositions comprising milvexian for use in methods of treating or preventing the conditions of the disclosure; and/or (ii) the use of milvexian or compositions comprising milvexian in the manufacture of a medicament for treating or preventing the conditions of the disclosure.

### Method of Use Aspects

Aspect 1. A method of preventing adverse cerebrovascular events or adverse cardiovascular events in a human patient with acute coronary syndrome, wherein the method comprises administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combinations thereof, wherein the pharmaceutical composition is administered twice daily.
Aspect 2. The method of aspect 1, wherein the antiplatelet therapy is a P2Y12 inhibitor.
Aspect 3. The method of aspect 2, wherein the P2Y12 inhibitor is clopidogrel, ticagrelor, or prasugrel.
Aspect 4. The method of any one of aspects 1-3, wherein the antiplatelet therapy is aspirin.
Aspect 5. The method of any one of aspects 1-4, wherein the human patient is treated with aspirin and clopidogrel combination therapy from day 1 to day 21, followed by aspirin monotherapy for at least 90 days.
Aspect 6. The method of aspect 5, wherein the patient is treated with aspirin and/or clopidogrel without milvexian dose adjustment.
Aspect 7. The method of any one of the preceding aspects, wherein the method is for primary prevention of adverse cerebrovascular events or adverse cardiovascular events.
Aspect 8. The method of any one of the preceding aspects, wherein the method is for secondary prevention of adverse cerebrovascular events or adverse cardiovascular events.
Aspect 9. The method of any one of aspects 1-8, wherein the adverse cerebrovascular events or adverse cardiovascular events comprises one or more of stroke, heart attack, or death.
Aspect 10. The method of any one of aspects 1-9, wherein the adverse cerebrovascular events or adverse cardiovascular events occur in an organ selected from heart, brain, limb, blood supply system, or vessel.
Aspect 11. The method of any one of aspects 1-10, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.
Aspect 12. The method of any one of aspects 1-11, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.
Aspect 13. The method of any one of aspects 1-12, wherein the adverse cerebrovascular events or adverse cardiovascular events are selected from the group consisting of arrhythmogenic cardiomyopathy (ACM), nonfatal myocardial infarction, ischemic stroke, and combinations thereof.
Aspect 14. The method of any one of aspects 1-11, wherein the adverse cerebrovascular events or adverse cardiovascular events comprises cardiovascular death.
Aspect 15. The method of any one of aspects 1-10, wherein the adverse cerebrovascular events or adverse cardiovascular events comprises arrhythmogenic cardiomyopathy (ACM).
Aspect 16. A method for preventing adverse cardiovascular events in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof,
   i. wherein the pharmaceutical composition is administered twice daily; and
   ii. wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; and transient ischemic attack (TIA).
Aspect 17. The method of aspect 16, wherein the adverse cardiovascular event is one or more of CV death, MI, or ischemic stroke.
Aspect 18. A method for reducing incidence rate of the one or more of adverse thrombotic event selected from new ischemic stroke, MI, or all-cause death in a human patient diagnosed with acute coronary syndrome, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof, wherein the pharmaceutical composition is administered twice daily.
Aspect 19. A method for preventing ischemic stroke in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof, wherein the pharmaceutical composition is administered twice daily.
Aspect 20. The method of aspect 19, wherein the clinical benefit of reducing the incidence rates of the ischemic stroke by the regimen is maintained throughout a treatment period of 90 days.
Aspect 21. The method of any one of the preceding aspects, wherein administration of the regimen reduces the patient's FXI clotting activity by about 7% to about 20% relative to baseline.
Aspect 22. The method of any one of the preceding aspects, wherein administration of the regimen results is a prolongation of activated partial thromboplastin time (aPTT) ranging from about 27% to about 64% relative to baseline.
Aspect 23. The method of any one of the preceding aspects, where the administration does not result in a statistically significant increase in major bleeding complications.
Aspect 24. The method of any one of the preceding aspects, wherein the pharmaceutical composition comprising milvexian (or pharmaceutically acceptable salt or solvate thereof) is a solid oral pharmaceutical composition.
Aspect 25. The method of aspect 24, wherein the solid oral pharmaceutical composition is a tablet.
Aspect 26. The method of aspect 25, wherein the tablet is an immediate release tablet.
Aspect 27. The method of aspect 25 or aspect 26, wherein the tablet has a disintegration time in water of less than 20 seconds.
Aspect 28. The method of any one of the preceding aspects, wherein the administration results in a milvexian plasma half-life ranging from about 13 hours to about 16 hours.
Aspect 29. The method of any one of the preceding aspects, wherein the administration results in milvexian plasma concentration reaching steady-state at about 3 days to 6 days.
Aspect 30. The method of any one of the preceding aspects, wherein the pharmaceutical composition comprising milvexian (or pharmaceutically acceptable salt or solvate thereof) is administered without regard to the timing of food intake.
Aspect 31. The method of any one of the preceding aspects, wherein the human patient is unable to swallow a tablet dosage form.
Aspect 32. The method of any one of aspects 25-31, wherein the tablet is dispersed in an aqueous medium to form an aqueous dispersion.
Aspect 33. The method of aspect 32, wherein the aqueous medium is water, saline, phosphate buffer, vegetable juice, or fruit juice, including, for example, apple sauce.
Aspect 34. The method of aspect 32 or aspect 33, wherein the aqueous dispersion is administered to the human patient via a nasogastric tube or spoon.
Aspect 35. The method of aspect any one of aspects 25-34, wherein the pharmaceutical composition is administered orally as an aqueous dispersion by dispersing the oral tablet in an aqueous medium in less than 1 minute.
Aspect 36. A method of preventing adverse cerebrovascular events or adverse cardiovascular events in a human patient with acute coronary syndrome, wherein the method comprises orally administering to the human patient a regimen comprising: (i) a pharmaceutical composition comprising 25 mg of milvexian (or pharmaceutically acceptable salt or solvate thereof); and (ii) an antiplatelet therapy; wherein the pharmaceutical composition is administered twice daily.
Aspect 37. The method of aspect 36, wherein the antiplatelet therapy is a P2Y12 inhibitor.
Aspect 38. The method of aspect 37, wherein the P2Y12 inhibitor is clopidogrel, ticagrelor, or prasugrel.
Aspect 39. The method of any one of aspects 36-38, wherein the antiplatelet therapy is aspirin.
Aspect 40. The method of any one of aspects 36-39, wherein the human patient is treated with aspirin and clopidogrel combination therapy from day 1 to day 21, followed by aspirin monotherapy for at least 90 days.
Aspect 41. The method of aspect 40, wherein the patient is treated with aspirin and/or clopidogrel without milvexian dose adjustment.
Aspect 42. The method of any one of aspects 36-41, wherein the method is for primary prevention of adverse cerebrovascular events or adverse cardiovascular events.
Aspect 43. The method of any one of aspects 36-42, wherein the method is for secondary prevention of adverse cerebrovascular events or adverse cardiovascular events.
Aspect 44. The method of any one of aspects 36-43, wherein the adverse cerebrovascular events or adverse cardiovascular events comprises one or more of stroke, heart attack, or death.
Aspect 45. The method of any one of aspects 36-44, wherein the adverse cerebrovascular events or adverse cardiovascular events occur in an organ selected from heart, brain, limb, blood supply system, or vessel.
Aspect 46. The method of any one of aspects 36-45, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.
Aspect 47. The method of any one of aspects 36-46, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.
Aspect 48. The method of any one of aspects 36-47, wherein the adverse cerebrovascular events or adverse cardiovascular events are selected from the group consisting of arrhythmogenic cardiomyopathy (ACM), nonfatal myocardial infarction, ischemic stroke, and combinations thereof.
Aspect 49. The method of any one of aspects 36-47, wherein the adverse cerebrovascular events or adverse cardiovascular events comprises cardiovascular death.
Aspect 50. The method of any one of aspects 36-49, wherein the adverse cerebrovascular events or adverse cardiovascular events comprises arrhythmogenic cardiomyopathy (ACM).
Aspect 51. A method for preventing adverse cardiovascular events in a human patient diagnosed with acute coronary syndrome, wherein the method comprises administering to the human patient a regimen comprising: (i) 25.0 mg of milvexian twice daily, and (ii) an antiplatelet therapy;
   wherein the adverse cardiovascular event is one or more of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; or transient ischemic attack (TIA); and
   wherein the method achieves at least one of the following clinical outcomes: (1) reaches a steady state plasma concentration profile of milvexian in about 6 days; (2) reaches a steady state plasma concentration profile of milvexian characterized by: (i) a steady state Cₘₐₓ ranging from about 217 ng/mL to about 475 ng/mL, (ii) a steady state mean (std) Cₘₐₓ of 346 (129) ng/mL, (iii) a steady state AUC₀₋₂₄ ranging from about about 4350 ng*h/mL to about 10230 ng*h/mL, or (iv) a steady state mean (std) AUC₀₋₂₄ of 7290 (2940) ng*h/mL; (3) a change from baseline in QTc in the patient of less than 10 millisecond, (4) no clinically relevant effects on any bleeding caused by milvexian exposure as measured by steady state AUC or Cₘₐₓ; (5) minimal impairment of hemostasis in the human patient, (6) no clinically meaningful change to prothrombin time, with a maximal mean percent change from baseline of approximately 5%; or (7) any combination of the aforementioned clinical outcomes.
Aspect 52. A method for preventing adverse cardiovascular events in a human patient diagnosed with acute coronary syndrome, wherein the method comprises administering to the human patient a regimen comprising: (i) 25.0 mg of milvexian twice daily, and (ii) an antiplatelet therapy;
   wherein the adverse cardiovascular event is one or more of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; or transient ischemic attack (TIA).
Aspect 53. A method for preventing all-cause mortality in a human patient diagnosed with acute coronary syndrome, wherein the method comprises administering to the human patient a regimen comprising: (i) 25.0 mg of milvexian twice daily, and (ii) an antiplatelet therapy.
Aspect 54. A method for preventing cardiovascular death in a human patient diagnosed with acute coronary syndrome, wherein the method comprises administering to the human patient a regimen comprising: (i) 25.0 mg of milvexian twice daily, and (ii) an antiplatelet therapy.
Aspect 55. A method for preventing myocardial infarction in a human patient diagnosed with acute coronary syndrome, wherein the method comprises administering to the human patient a regimen comprising: (i) 25.0 mg of milvexian twice daily, and (ii) an antiplatelet therapy.
Aspect 56. A method for preventing stroke in a human patient diagnosed with acute coronary syndrome, wherein the method comprises administering to the human patient a regimen comprising: (i) 25.0 mg of milvexian twice daily, and (ii) an antiplatelet therapy.
Aspect 57. A method for preventing ischemic stroke in a human patient diagnosed with acute coronary syndrome, wherein the method comprises administering to the human patient a regimen comprising: (i) 25.0 mg of milvexian twice daily, and (ii) an antiplatelet therapy.
Aspect 58. A method for preventing ischemic stroke in a human patient diagnosed with acute coronary syndrome, wherein the method comprises administering to the human patient a regimen comprising: (i) 25.0 mg of milvexian twice daily, and (ii) an antiplatelet therapy.
Aspect 59. A method for preventing unstable angina in a human patient diagnosed with acute coronary syndrome, wherein the method comprises administering to the human patient a regimen comprising: (i) 25.0 mg of milvexian twice daily, and (ii) an antiplatelet therapy.
Aspect 60. A method for preventing acute limb inschemia in a human patient diagnosed with acute coronary syndrome, wherein the method comprises administering to the human patient a regimen comprising: (i) 25.0 mg of milvexian twice daily, and (ii) an antiplatelet therapy.
Aspect 61. A method for preventing major vascular (non-traumatic) limb amputation in a human patient diagnosed with acute coronary syndrome, wherein the method comprises administering to the human patient a regimen comprising: (i) 25.0 mg of milvexian twice daily, and (ii) an antiplatelet therapy.
Aspect 62. A method for preventing symptomatic venous thromboembolism in a human patient diagnosed with acute coronary syndrome, wherein the method comprises administering to the human patient a regimen comprising: (i) 25.0 mg of milvexian twice daily, and (ii) an antiplatelet therapy.
Aspect 63. A method for preventing pulmonary embolism in a human patient diagnosed with acute coronary syndrome, wherein the method comprises administering to the human patient a regimen comprising: (i) 25.0 mg of milvexian twice daily, and (ii) an antiplatelet therapy.
Aspect 64. A method for preventing deep vein thrombosis in a human patient diagnosed with acute coronary syndrome, wherein the method comprises administering to the human patient a regimen comprising: (i) 25.0 mg of milvexian twice daily, and (ii) an antiplatelet therapy.
Aspect 65. A method for preventing deep vein thrombosis in a human patient diagnosed with acute coronary syndrome, wherein the method comprises administering to the human patient a regimen comprising: (i) 25.0 mg of milvexian twice daily, and (ii) an antiplatelet therapy.
Aspect 66. A method for preventing ischemia-driven coronary revascularization in a human patient diagnosed with acute coronary syndrome, wherein the method comprises administering to the human patient a regimen comprising: (i) 25.0 mg of milvexian twice daily, and (ii) an antiplatelet therapy.
Aspect 67. A method for preventing stent thrombosis in a human patient diagnosed with acute coronary syndrome, wherein the method comprises administering to the human patient a regimen comprising: (i) 25.0 mg of milvexian twice daily, and (ii) an antiplatelet therapy.
Aspect 68. A method for preventing hospitalization for any reason, categorized as (a) planned or unplanned, (b) for arterial or venous thrombotic event or neither in a human patient diagnosed with acute coronary syndrome, wherein the method comprises administering to the human patient a regimen comprising: (i) 25.0 mg of milvexian twice daily, and (ii) an antiplatelet therapy.
Aspect 69. A method for preventing transient ischemic attack in a human patient diagnosed with acute coronary syndrome, wherein the method comprises administering to the human patient a regimen comprising: (i) 25.0 mg of milvexian twice daily, and (ii) an antiplatelet therapy.
Aspect 70. A method of preventing adverse cerebrovascular events in a human patient with acute coronary syndrome, wherein the method comprises orally administering twice daily to the human patient a regimen comprising: (i) 25 mg of milvexian; and (ii) an antiplatelet therapy.
Aspect 71. A method of preventing adverse cardiovascular events in a human patient with acute coronary syndrome, wherein the method comprises orally administering twice daily to the human patient a regimen comprising: (i) 25 mg of milvexian; and (ii) an antiplatelet therapy.
Aspect 72. A method of preventing adverse cerebrovascular events in a human patient with acute coronary syndrome, wherein the method comprises orally administering to the human patient a regimen comprising: (i) a pharmaceutical composition comprising 25 mg of milvexian and a pharmaceutically acceptable excipient; and (ii) an antiplatelet therapy; wherein the pharmaceutical composition is administered twice daily.
Aspect 73. A method of preventing adverse cardiovascular events in a human patient with acute coronary syndrome, wherein the method comprises administering to the human patient a regimen comprising: (i) a pharmaceutical composition comprising 25 mg of milvexian and a pharmaceutically acceptable excipient; and (ii) an antiplatelet therapy; wherein the pharmaceutical composition is administered twice daily.
Aspect 74. The method of any one of aspects of 70-73, wherein the adverse cerebrovascular events or cardiovascular events comprises ischemic stroke.
Aspect 75. The method any one of any one of aspects of 70-74, wherein the adverse cerebrovascular events or cardiovascular events comprises one or more of stroke, heart attack, or death.
Aspect 76. The method of any one of any one of aspects 70-75, wherein the adverse cerebrovascular events or adverse cardiovascular events occur in an organ selected from heart, brain, limb, blood supply system, or vessel.
Aspect 77. The method of any one of any one of aspects 70-76, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.
Aspect 78. The method of any one of any one of aspects 70-77, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.
Aspect 79. The method of any one of aspects 70-78, wherein the adverse cerebrovascular events or adverse cardiovascular events are selected from the group consisting of arrhythmogenic cardiomyopathy (ACM), nonfatal myocardial infarction, ischemic stroke, and combinations thereof.
Aspect 80. The method of any one of aspects 70-79, wherein the adverse cerebrovascular events or adverse cardiovascular events comprises cardiovascular death.
Aspect 81. The method of any one of aspects 70-80, wherein the adverse cerebrovascular events or adverse cardiovascular events comprises arrhythmogenic cardiomyopathy (ACM).
Aspect 82. A method of preventing ischemic stroke in a human patient with acute coronary syndrome, wherein the method comprises orally administering twice daily to the human patient a regimen comprising: (i) 25 mg of milvexian; and (ii) an antiplatelet therapy.
Aspect 83. The method of any one of the preceding aspects, wherein the administration results in a steady state plasma concentration profile of milvexian in about 3 days.
Aspect 84. The method of any one of the preceding aspects, wherein the administration results in a steady state plasma concentration profile of milvexian characterized by:
   (i) a steady state Cₘₐₓ ranging from about 217 ng/mL to about 475 ng/mL,
   (ii) a steady state mean (std) Cₘₐₓ of 346 (129) ng/mL,
   (iii) a steady state AUC₀₋₂₄ ranging from about 4350 ng*h/mL to about 10230 ng*h/mL, and
   (iv) a steady state mean (std) AUC₀₋₂₄ of 7290 (2940) ng*h/mL.
Aspect 85. The method of any one of the preceding aspects, wherein the administration results in no clinically significant QTc interval prolongation.
Aspect 86. The method of any one of the preceding aspects, wherein the administration results in no clinically relevant effects on any bleeding caused by milvexian exposure as measured by steady state AUC or Cₘₐₓ.
Aspect 87. The method of any one of the preceding aspects, wherein the administration results in minimal impairment of hemostasis in the human patient.
Aspect 88. The method of any one of the preceding aspects, wherein the regimen comprises an immediate release tablet.
Aspect 89. The method of aspect 88, wherein the immediate release tablet has a disintegration time in water of less than 20 seconds at 37° C
Aspect 90. The method of any one of the preceding aspects, wherein the administration results in a milvexian plasma terminal half-life ranging from about 13 hours to about 16 hours.
Aspect 91. The method of any one of the preceding aspects, wherein the regimen is administered without regard to the timing of food intake.
Aspect 92. The method of any one of aspects 88-91, wherein the immediate release tablet is dispersed in an aqueous medium to form an aqueous dispersion.
Aspect 93. The method of aspect 92, wherein the aqueous medium comprises water or apple sauce.
Aspect 94. The method of any one of the preceding aspects, wherein the antiplatelet therapy comprises administering a single antiplatelet therapy (SAPT) for a period ranging from about 1 month to about 36 months.
Aspect 95. The method of aspect 94, wherein the antiplatelet therapy comprises administering a SAPT for a period ranging from about 3 months to about 12 months.
Aspect 96. The method of aspect 94, wherein the antiplatelet therapy comprises administering a SAPT for a period ranging from about 6 months to about 12 months.
Aspect 97. The method of aspect 94, wherein the antiplatelet therapy comprises administering a SAPT for 3 months.
Aspect 98. The method of aspect 94, wherein the antiplatelet therapy comprises administering a SAPT for 6 months.
Aspect 99. The method of aspect 94, wherein the antiplatelet therapy comprises administering a SAPT for 12 months.
Aspect 100. The method of aspect 94, wherein the antiplatelet therapy comprises administering a SAPT for 24 months.
Aspect 101. The method of any one of aspects 94-100, wherein the SAPT is selected from aspirin, and a P2Y12 inhibitor.
Aspect 102. The method of any one of aspects 94-101, wherein the SAPT comprises aspirin.
Aspect 103. The method of aspect 102, wherein the aspirin is administered once daily.
Aspect 104. The method of aspect 102 wherein the aspirin is administered once daily for a period of ranging from about 3 months to about 3 years.
Aspect 105. The method of aspect 102, wherein the aspirin is administered once daily for 3 months.
Aspect 106. The method of aspect 102, wherein the aspirin is administered once daily for 6 months.
Aspect 107. The method of aspect 102, wherein the aspirin is administered once daily for 12 months.
Aspect 108. The method of aspect 102, wherein the aspirin is administered once daily for 24 months.
Aspect 109. The method of aspect 102, wherein the aspirin is administered once daily for 36 months.
Aspect 110. The method of aspect 102, wherein the aspirin is administered twice daily.
Aspect 111. The method of aspect 102, wherein the aspirin is administered once daily at an amount of 75 mg to 100 mg.
Aspect 112. The method of aspect 102, wherein the aspirin is administered once daily at an amount of 75 mg to 100 mg for a period of 12 months.
Aspect 113. The method of any one of aspects 94-101, wherein the SAPT comprises a P2Y12 inhibitor.
Aspect 114. The method of any one of aspects 94-101, wherein the SAPT comprises a P2Y12 inhibitor selected from prasugrel, clopidogrel, selatogrel, or ticagrelor.
Aspect 115. The method of aspect 113 or 114, wherein the P2Y12 inhibitor is administered for a period ranging from about 3 months to about 6 months.
Aspect 116. The method of aspect 113 or 114, wherein the P2Y12 inhibitor comprises clopidogrel.
Aspect 117. The method of aspect 113 or 114, wherein the P2Y12 inhibitor comprises ticagrelor.
Aspect 118. The method of aspect 113 or 114, wherein the P2Y12 inhibitor comprises ticagrelor administered at 90 mg twice daily.
Aspect 119. The method of aspect 113 or 114, wherein the P2Y12 inhibitor comprises ticagrelor administered at 90 mg twice daily for a period of 3 years.
Aspect 120. The method of aspect 113 or 114, wherein the P2Y12 inhibitor comprises ticagrelor administered first followed by aspirin monotherapy administered at 20 mg twice daily.
Aspect 121. The method of any one of aspects 113 or 114, wherein the SAPT comprises aspirin monotherapy administered first for a period of ranging from 30 days to 90 days followed by clopidogrel monotherapy.
Aspect 122. The method of any one of aspects 113 or 114, wherein the SAPT comprises aspirin monotherapy administered first for a period of 60 days followed by clopidogrel monotherapy.
Aspect 123. The method of any one of aspects 94-122, wherein the SAPT comprises aspirin monotherapy administered first for a period of 90 days followed by clopidogrel monotherapy.
Aspect 124. The method of any one of aspects 94-123, wherein the SAPT comprises aspirin monotherapy administered first followed by clopidogrel monotherapy for a period of at least 2 months.
Aspect 125. The method of any one of aspects 94-123, wherein the SAPT comprises aspirin monotherapy administered first for a period ranging from 30 days to 90 days followed by clopidogrel monotherapy for a period of at least 2 months.
Aspect 126. The method of any one of aspects 1-93, wherein the antiplatelet therapy comprises a dual antiplatelet therapy (DAPT).
Aspect 127. The method of aspect of 126, wherein the DAPT comprises aspirin and prasugrel.
Aspect 128. The method of aspect of 126, wherein the DAPT comprises aspirin and prasugrel administered for 12 months.
Aspect 129. The method of aspect of 126, wherein the DAPT comprises aspirin and clopidogrel.
Aspect 130. The method of aspect of 126, wherein the DAPT comprises aspirin and clopidogrel administered for a period of 21 days to 12 months.
Aspect 131. The method of aspect of 126, wherein the DAPT comprises aspirin and clopidogrel administered for a period of 21 days.
Aspect 132. The method of aspect of 126, wherein the DAPT comprises aspirin and clopidogrel administered for a period of 6 months.
Aspect 133. The method of aspect of 126, wherein the DAPT comprises aspirin and clopidogrel administered for a period of 12 months.
Aspect 134. The method of aspect of 126, wherein the DAPT comprises aspirin and ticagrelor.
Aspect 135. The method of aspect of 126, wherein the DAPT comprises aspirin and ticagrelor administered for 12 months.
Aspect 136. The method of aspect 126, wherein the antiplatelet therapy comprises a DAPT consisting of aspirin administered once daily at 75 mg to 100 mg and a P2Y12 inhibitor.
Aspect 137. The method of aspect 126, wherein the antiplatelet therapy comprises a DAPT administered first followed by administering a SAPT.
Aspect 138. The method of aspect 126, wherein the antiplatelet therapy comprises a DAPT administered first followed by administering aspirin monotherapy.
Aspect 139. The method of aspect 126, wherein the antiplatelet therapy comprises a DAPT consisting of aspirin 75-100 mg once daily and a P2Y12 inhibitor administered for a first period of 12 months followed by administering aspirin monotherapy for a second period of 6 months.
Aspect 140. The method of aspect 126, wherein the antiplatelet therapy comprises a DAPT consisting of aspirin 75-100 mg once daily and a P2Y12 inhibitor administered for a first period of 21 days followed by administering aspirin monotherapy for a second period of 6 months.
Aspect 141. The method of aspect 126, wherein the antiplatelet therapy comprises a DAPT consisting of aspirin 75-100 mg once daily and clopidogrel 75 mg once daily administered for a first period of 21 days followed by administering aspirin monotherapy for a second period of 6 months.
Aspect 142. The method of aspect 126, wherein the antiplatelet therapy comprises a DAPT consisting of aspirin 75-100 mg once daily and a P2Y12 inhibitor administered for a first period of 21 days followed by administering aspirin monotherapy for a second period of 12 months.
Aspect 143. The method of aspect 126, wherein the antiplatelet therapy comprises a DAPT consisting of aspirin 75-100 mg once daily and ticagrelor 90 mg twice daily.
Aspect 144. The method of aspect 126, wherein the antiplatelet therapy comprises a DAPT consisting of aspirin 75-100 mg once daily and ticagrelor 90 mg twice daily administered for a period of 12 months.
Aspect 145. The method of aspect 126, wherein the antiplatelet therapy comprises a DAPT consisting of aspirin 81-100 mg once daily and ticagrelor 90 mg twice daily.
Aspect 146. The method of aspect 126, wherein the antiplatelet therapy comprises a DAPT consisting of aspirin 81-100 mg once daily and ticagrelor 90 mg twice daily administered for 3 years.
Aspect 147. The method of aspect 126, wherein the antiplatelet therapy comprises a DAPT consisting of aspirin 75-100 mg once daily and ticagrelor 90 mg twice daily administered for a first period of 12 months followed by administering aspirin monotherapy 75-100 mg once daily for a second period of 12 months.
Aspect 148. The method of aspect 126, wherein the antiplatelet therapy comprises a DAPT consisting of aspirin 75-100 mg once daily and ticagrelor 90 mg twice daily administered for a first period of 12 months followed by administering ticagrelor 90 mg twice daily for a second period of 23 months.
Aspect 149. The method of any one of the previous aspects, wherein the clinical benefit of reducing the incidence rates of the ischemic stroke by the regimen is maintained throughout a treatment period of 90 days.
Aspect 150. The method of any one of the previous aspects, wherein the clinical benefit of reducing the incidence rates of the ischemic stroke is maintained throughout a treatment period of 180 days.
Aspect 151. The method of any one of the previous aspects, wherein the clinical benefit of reducing the incidence rates of the ischemic stroke is maintained throughout a treatment period of 12 months.
Aspect 152. The method of any one of the preceding aspects, wherein administration of the regimen reduces the patient's FXI clotting activity by about 7% to about 20% relative to baseline.
Aspect 153. The method of any one of the preceding aspects, wherein administration of the regimen results is a prolongation of activated partial thromboplastin time (aPTT) ranging from about 27% to about 64% relative to baseline.
Aspect 154. The method of any one of the preceding aspects, where the administration does not result in a statistically significant increase in major bleeding complications.
Aspect 155. The method of any one of the preceding aspects, wherein the pharmaceutical composition comprising milvexian (or pharmaceutically acceptable salt or solvate thereof) is a solid oral pharmaceutical composition.
Aspect 156. The method of aspect 155, wherein the solid oral pharmaceutical composition is a tablet.
Aspect 157. The method of aspect 156, wherein the tablet is an immediate release tablet.
Aspect 158. The method of any one of the preceding aspects, wherein the human patient is unable to swallow a tablet dosage form.
Aspect 159. The method of any one of aspects 156-158, wherein the tablet is dispersed in an aqueous medium to form an aqueous dispersion.
Aspect 160. The method of aspect 159, wherein the aqueous medium is water, saline, phosphate buffer, vegetable juice, or fruit juice, including, for example, apple sauce.
Aspect 161. The method of aspect 159 or aspect 160, wherein the aqueous dispersion is administered to the human patient via a nasogastric tube or spoon.
Aspect 162. The method of any one of the preceding aspects, wherein the pharmaceutical composition is administered orally as an aqueous dispersion by dispersing the oral tablet in an aqueous medium in less than 1 minute.
Aspect 163. The method of any one of the preceding aspects, wherein the pharmaceutical composition comprises an amorphous form of milvexian admixed with a polymer.
Aspect 164. The method of any one of the preceding aspects, wherein the pharmaceutical composition comprises a spray dried amorphous solid dispersion consisting of 75% w/w milvexian and 25% w/w of HPMC-AS.
Aspect 165. The method of any one of the preceding aspects, wherein the method results in no clinically significant QTc interval prolongation.

### Milvexian for Use Aspects

Aspect 1. Milvexian for use in a method of preventing adverse cerebrovascular events or adverse cardiovascular events in a human patient with acute coronary syndrome, wherein the method comprises administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combinations thereof, wherein the pharmaceutical composition is administered twice daily.
Aspect 2. The milvexian for use of aspect 1, wherein the antiplatelet therapy is a P2Y12 inhibitor.
Aspect 3. The milvexian for use of aspect 2, wherein the P2Y12 inhibitor is clopidogrel, ticagrelor, or prasugrel.
Aspect 4. The milvexian for use of any one of aspects 1-3, wherein the antiplatelet therapy is aspirin.
Aspect 5. The milvexian for use of any one of aspects 1-4, wherein the human patient is treated with aspirin and clopidogrel combination therapy from day 1 to day 21, followed by aspirin monotherapy for at least 90 days.
Aspect 6. The milvexian for use of aspect 5, wherein the patient is treated with aspirin and/or clopidogrel without milvexian dose adjustment.
Aspect 7. The milvexian for use of any one of the preceding aspects, wherein the milvexian for use is for primary prevention of adverse cerebrovascular events or adverse cardiovascular events.
Aspect 8. The milvexian for use of any one of the preceding aspects, wherein the milvexian for use is for secondary prevention of adverse cerebrovascular events or adverse cardiovascular events.
Aspect 9. The milvexian for use of any one of aspects 1-8, wherein the adverse cerebrovascular events or adverse cardiovascular events comprises one or more of stroke, heart attack, or death.
Aspect 10. The milvexian for use of any one of aspects 1-9, wherein the adverse cerebrovascular events or adverse cardiovascular events occur in an organ selected from heart, brain, limb, blood supply system, or vessel.
Aspect 11. The milvexian for use of any one of aspects 1-10, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.
Aspect 12. The milvexian for use of any one of aspects 1-11, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.
Aspect 13. The milvexian for use of any one of aspects 1-12, wherein the adverse cerebrovascular events or adverse cardiovascular events are selected from the group consisting of arrhythmogenic cardiomyopathy (ACM), nonfatal myocardial infarction, ischemic stroke, and combinations thereof.
Aspect 14. The milvexian for use of any one of aspects 1-11, wherein the adverse cerebrovascular events or adverse cardiovascular events comprises cardiovascular death.
Aspect 15. The milvexian for use of any one of aspects 1-10, wherein the adverse cerebrovascular events or adverse cardiovascular events comprises arrhythmogenic cardiomyopathy (ACM).
Aspect 16. Milvexian for use in a method for preventing adverse cardiovascular events in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof,
   i. wherein the pharmaceutical composition is administered twice daily; and
   ii. wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; and transient ischemic attack (TIA).
Aspect 17. The milvexian for use of aspect 16, wherein the adverse cardiovascular event is one or more of CV death, MI, or ischemic stroke.
Aspect 18. Milvexian for use in a method for reducing incidence rate of the one or more of adverse thrombotic event selected from new ischemic stroke, MI, or all-cause death in a human patient diagnosed with acute coronary syndrome, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof, wherein the pharmaceutical composition is administered twice daily.
Aspect 19. Milvexian for use in a method for preventing ischemic stroke in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.
Aspect 20. The milvexian for use of aspect 18 or aspect 19, wherein the clinical benefit of reducing the incidence rates of the ischemic stroke by the regimen is maintained throughout a treatment period of 90 days.
Aspect 21. The milvexian for use of any one of the preceding aspects, wherein administration of the regimen reduces the patient's FXI clotting activity by about 7% to about 20% relative to baseline.
Aspect 22. The milvexian for use of any one of the preceding aspects, wherein administration of the regimen results is a prolongation of activated partial thromboplastin time (aPTT) ranging from about 27% to about 64% relative to baseline.
Aspect 23. The milvexian for use of any one of the preceding aspects, where the administration does not result in a statistically significant increase in major bleeding complications.
Aspect 24. The milvexian for use of any one of the preceding aspects, wherein the pharmaceutical composition comprising milvexian (or pharmaceutically acceptable salt or solvate thereof) is a solid oral pharmaceutical composition.
Aspect 25. The milvexian for use of aspect 24, wherein the solid oral pharmaceutical composition is a tablet.
Aspect 26. The milvexian for use of aspect 25, wherein the tablet is an immediate release tablet.
Aspect 27. The milvexian for use of aspect 25 or aspect 26, wherein the tablet has a disintegration time in water of less than 20 seconds.
Aspect 28. The milvexian for use of any one of the preceding aspects, wherein the administration results in a milvexian plasma half-life ranging from about 13 hours to about 16 hours.
Aspect 29. The milvexian for use of any one of the preceding aspects, wherein the administration results in milvexian plasma concentration reaching steady-state at about 3 days to 6 days.
Aspect 30. The milvexian for use of any one of the preceding aspects, wherein the pharmaceutical composition comprising milvexian (or pharmaceutically acceptable salt or solvate thereof) is administered without regard to the timing of food intake.
Aspect 31. The milvexian for use of any one of the preceding aspects, wherein the human patient is unable to swallow a tablet dosage form.
Aspect 32. The milvexian for use of any one of aspects 25-31, wherein the tablet is dispersed in an aqueous medium to form an aqueous dispersion.
Aspect 33. The milvexian for use of aspect 32, wherein the aqueous medium is water, saline, phosphate buffer, vegetable juice, or fruit juice, including, for example, apple sauce.
Aspect 34. The milvexian for use of aspect 32 or aspect 33, wherein the aqueous dispersion is administered to the human patient via a nasogastric tube or spoon.
Aspect 35. The milvexian for use of aspect any one of aspects 25-34, wherein the pharmaceutical composition is administered orally as an aqueous dispersion by dispersing the oral tablet in an aqueous medium in less than 1 minute.
Aspect 36. Milvexian for use in a method of preventing adverse cerebrovascular events or adverse cardiovascular events in a human patient with acute coronary syndrome, wherein the method comprises orally administering to the human patient a regimen comprising: (i) a pharmaceutical composition comprising 25 mg of milvexian (or pharmaceutically acceptable salt or solvate thereof); and (ii) an antiplatelet therapy; wherein the pharmaceutical composition is administered twice daily.
Aspect 37. The milvexian for use of aspect 36, wherein the antiplatelet therapy is a P2Y12 inhibitor.
Aspect 38. The milvexian for use of aspect 37, wherein the P2Y12 inhibitor is clopidogrel, ticagrelor, or prasugrel.
Aspect 39. The milvexian for use of any one of aspects 36-38, wherein the antiplatelet therapy is aspirin.
Aspect 40. The milvexian for use of any one of aspects 36-39, wherein the human patient is treated with aspirin and clopidogrel combination therapy from day 1 to day 21, followed by aspirin monotherapy for at least 90 days.
Aspect 41. The milvexian for use of aspect 40, wherein the patient is treated with aspirin and/or clopidogrel without milvexian dose adjustment.
Aspect 42. The milvexian for use of any one of aspects 36-41, wherein the milvexian for use is for primary prevention of adverse cerebrovascular events or adverse cardiovascular events.
Aspect 43. The milvexian for use of any one of aspects 36-42, wherein the milvexian for use is for secondary prevention of adverse cerebrovascular events or adverse cardiovascular events.
Aspect 44. The milvexian for use of any one of aspects 36-43, wherein the adverse cerebrovascular events or adverse cardiovascular events comprises one or more of stroke, heart attack, or death.
Aspect 45. The milvexian for use of any one of aspects 36-44, wherein the adverse cerebrovascular events or adverse cardiovascular events occur in an organ selected from heart, brain, limb, blood supply system, or vessel.
Aspect 46. The milvexian for use of any one of aspects 36-45, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.
Aspect 47. The milvexian for use of any one of aspects 36-46, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.
Aspect 48. The milvexian for use of any one of aspects 36-47, wherein the adverse cerebrovascular events or adverse cardiovascular events are selected from the group consisting of arrhythmogenic cardiomyopathy (ACM), nonfatal myocardial infarction, ischemic stroke, and combinations thereof.
Aspect 49. The milvexian for use of any one of aspects 36-47, wherein the adverse cerebrovascular events or adverse cardiovascular events comprises cardiovascular death.
Aspect 50. The milvexian for use of any one of aspects 36-48, wherein the adverse cerebrovascular events or adverse cardiovascular events comprises arrhythmogenic cardiomyopathy (ACM).
Aspect 51. Milvexian for use in a method for preventing adverse cardiovascular events in a human patient diagnosed with acute coronary syndrome, wherein the method comprises administering to the human patient a regimen comprising: (i) 25.0 mg of milvexian twice daily, and (ii) an antiplatelet therapy;
wherein the adverse cardiovascular event is one or more of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; or transient ischemic attack (TIA); and
wherein the method achieves at least one of the following clinical outcomes: (1) reaches a steady state plasma concentration profile of milvexian in about 6 days; (2) reaches a steady state plasma concentration profile of milvexian characterized by: (i) a steady state Cₘₐₓ ranging from about 217 ng/mL to about 475 ng/mL, (ii) a steady state mean (std) Cₘₐₓ of 346 (129) ng/mL, (iii) a steady state AUC₀₋₂₄ ranging from about about 4350 ng*h/mL to about 10230 ng*h/mL, or (iv) a steady state mean (std) AUC₀₋₂₄ of 7290 (2940) ng*h/mL; (3) a change from baseline in QTc in the patient of less than 10 millisecond, (4) no clinically relevant effects on any bleeding caused by milvexian exposure as measured by steady state AUC or Cₘₐₓ; (5) minimal impairment of hemostasis in the human patient, (6) no clinically meaningful change to prothrombin time, with a maximal mean percent change from baseline of approximately 5%; or (7) any combination of the aforementioned clinical outcomes.
Aspect 52. Milvexian for use in a method for preventing adverse cardiovascular events in a human patient diagnosed with acute coronary syndrome, wherein the method comprises administering to the human patient a regimen comprising: (i) 25.0 mg of milvexian twice daily, and (ii) an antiplatelet therapy;
wherein the adverse cardiovascular event is one or more of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; or transient ischemic attack (TIA).
Aspect 53. Milvexian for use in a method for preventing all-cause mortality in a human patient diagnosed with acute coronary syndrome, wherein the method comprises administering to the human patient a regimen comprising: (i) 25.0 mg of milvexian twice daily, and (ii) an antiplatelet therapy.
Aspect 54. Milvexian for use in a method for preventing cardiovascular death in a human patient diagnosed with acute coronary syndrome, wherein the method comprises administering to the human patient a regimen comprising: (i) 25.0 mg of milvexian twice daily, and (ii) an antiplatelet therapy.
Aspect 55. Milvexian for use in a method for preventing myocardial infarction in a human patient diagnosed with acute coronary syndrome, wherein the method comprises administering to the human patient a regimen comprising: (i) 25.0 mg of milvexian twice daily, and (ii) an antiplatelet therapy.
Aspect 56. Milvexian for use in a method for preventing stroke in a human patient diagnosed with acute coronary syndrome, wherein the method comprises administering to the human patient a regimen comprising: (i) 25.0 mg of milvexian twice daily, and (ii) an antiplatelet therapy.
Aspect 57. Milvexian for use in a method for preventing ischemic stroke in a human patient diagnosed with acute coronary syndrome, wherein the method comprises administering to the human patient a regimen comprising: (i) 25.0 mg of milvexian twice daily, and (ii) an antiplatelet therapy.
Aspect 58. Milvexian for use in a method for preventing ischemic stroke in a human patient diagnosed with acute coronary syndrome, wherein the method comprises administering to the human patient a regimen comprising: (i) 25.0 mg of milvexian twice daily, and (ii) an antiplatelet therapy.
Aspect 59. Milvexian for use in a method for preventing unstable angina in a human patient diagnosed with acute coronary syndrome, wherein the method comprises administering to the human patient a regimen comprising: (i) 25.0 mg of milvexian twice daily, and (ii) an antiplatelet therapy.
Aspect 60. Milvexian for use in a method for preventing acute limb ischemia in a human patient diagnosed with acute coronary syndrome, wherein the method comprises administering to the human patient a regimen comprising: (i) 25.0 mg of milvexian twice daily, and (ii) an antiplatelet therapy.
Aspect 61. Milvexian for use in a method for preventing major vascular (non-traumatic) limb amputation in a human patient diagnosed with acute coronary syndrome, wherein the method comprises administering to the human patient a regimen comprising: (i) 25.0 mg of milvexian twice daily, and (ii) an antiplatelet therapy.
Aspect 62. Milvexian for use in a method for preventing symptomatic venous thromboembolism in a human patient diagnosed with acute coronary syndrome, wherein the method comprises administering to the human patient a regimen comprising: (i) 25.0 mg of milvexian twice daily, and (ii) an antiplatelet therapy.
Aspect 63. Milvexian for use in a method for preventing pulmonary embolism in a human patient diagnosed with acute coronary syndrome, wherein the method comprises administering to the human patient a regimen comprising: (i) 25.0 mg of milvexian twice daily, and (ii) an antiplatelet therapy.
Aspect 64. Milvexian for use in a method for preventing deep vein thrombosis in a human patient diagnosed with acute coronary syndrome, wherein the method comprises administering to the human patient a regimen comprising: (i) 25.0 mg of milvexian twice daily, and (ii) an antiplatelet therapy.
Aspect 65. Milvexian for use in a method for preventing deep vein thrombosis in a human patient diagnosed with acute coronary syndrome, wherein the method comprises administering to the human patient a regimen comprising: (i) 25.0 mg of milvexian twice daily, and (ii) an antiplatelet therapy.
Aspect 66. Milvexian for use in a method for preventing ischemia-driven coronary revascularization in a human patient diagnosed with acute coronary syndrome, wherein the method comprises administering to the human patient a regimen comprising: (i) 25.0 mg of milvexian twice daily, and (ii) an antiplatelet therapy.
Aspect 67. Milvexian for use in a method for preventing stent thrombosis in a human patient diagnosed with acute coronary syndrome, wherein the method comprises administering to the human patient a regimen comprising: (i) 25.0 mg of milvexian twice daily, and (ii) an antiplatelet therapy.
Aspect 68. Milvexian for use in a method for preventing hospitalization for any reason, categorized as (a) planned or unplanned, (b) for arterial or venous thrombotic event or neither in a human patient diagnosed with acute coronary syndrome, wherein the method comprises administering to the human patient a regimen comprising: (i) 25.0 mg of milvexian twice daily, and (ii) an antiplatelet therapy.
Aspect 69. Milvexian for use in a method for preventing transient ischemic attack in a human patient diagnosed with acute coronary syndrome, wherein the method comprises administering to the human patient a regimen comprising: (i) 25.0 mg of milvexian twice daily, and (ii) an antiplatelet therapy.
Aspect 70. Milvexian for use in a method of preventing adverse cerebrovascular events in a human patient with acute coronary syndrome, wherein the method comprises orally administering twice daily to the human patient a regimen comprising: (i) 25 mg of milvexian; and (ii) an antiplatelet therapy.
Aspect 71. Milvexian for use in a method of preventing adverse cardiovascular events in a human patient with acute coronary syndrome, wherein the method comprises orally administering twice daily to the human patient a regimen comprising: (i) 25 mg of milvexian; and (ii) an antiplatelet therapy.
Aspect 72. Milvexian for use in a method of preventing adverse cerebrovascular events in a human patient with acute coronary syndrome, wherein the method comprises orally administering to the human patient a regimen comprising: (i) a pharmaceutical composition comprising 25 mg of milvexian and a pharmaceutically acceptable excipient; and (ii) an antiplatelet therapy; wherein the pharmaceutical composition is administered twice daily.
Aspect 73. Milvexian for use in a method of preventing adverse cardiovascular events in a human patient with acute coronary syndrome, wherein the method comprises administering to the human patient a regimen comprising: (i) a pharmaceutical composition comprising 25 mg of milvexian and a pharmaceutically acceptable excipient; and (ii) an antiplatelet therapy; wherein the pharmaceutical composition is administered twice daily.
Aspect 74. The milvexian for use of any one of aspects of 70-73, wherein the adverse cerebrovascular events or cardiovascular events comprises ischemic stroke.
Aspect 75. The milvexian for use any one of any one of aspects of 70-74, wherein the adverse cerebrovascular events or cardiovascular events comprises one or more of stroke, heart attack, or death.
Aspect 76. The milvexian for use of any one of any one of aspects 70-75, wherein the adverse cerebrovascular events or adverse cardiovascular events occur in an organ selected from heart, brain, limb, blood supply system, or vessel.
Aspect 77. The milvexian for use of any one of any one of aspects 70-76, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.
Aspect 78. The milvexian for use of any one of any one of aspects 70-77, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.
Aspect 79. The milvexian for use of any one of aspects 70-78, wherein the adverse cerebrovascular events or adverse cardiovascular events are selected from the group consisting of arrhythmogenic cardiomyopathy (ACM), nonfatal myocardial infarction, ischemic stroke, and combinations thereof.
Aspect 80. The milvexian for use of any one of aspects 70-79, wherein the adverse cerebrovascular events or adverse cardiovascular events comprises cardiovascular death.
Aspect 81. The milvexian for use of any one of aspects 70-80, wherein the adverse cerebrovascular events or adverse cardiovascular events comprises arrhythmogenic cardiomyopathy (ACM).
Aspect 82. Milvexian for use in a method of preventing ischemic stroke in a human patient with acute coronary syndrome, wherein the method comprises orally administering twice daily to the human patient a regimen comprising: (i) 25 mg of milvexian; and (ii) an antiplatelet therapy.
Aspect 83. The milvexian for use of any one of the preceding aspects, wherein the administration results in a steady state plasma concentration profile of milvexian in about 3 days.
Aspect 84. The milvexian for use of any one of the preceding aspects, wherein the administration results in a steady state plasma concentration profile of milvexian characterized by:
(i) a steady state Cₘₐₓ ranging from about 217 ng/mL to about 475 ng/mL,
(ii) a steady state mean (std) Cₘₐₓ of 346 (129) ng/mL,
(iii) a steady state AUC₀₋₂₄ ranging from about 4350 ng*h/mL to about 10230 ng*h/mL, and
(iv) a steady state mean (std) AUC₀₋₂₄ of 7290 (2940) ng*h/mL.
Aspect 85. The milvexian for use of any one of the preceding aspects, wherein the administration results in no clinically significant QTc interval prolongation.
Aspect 86. The milvexian for use of any one of the preceding aspects, wherein the administration results in no clinically relevant effects on any bleeding caused by milvexian exposure as measured by steady state AUC or Cₘₐₓ.
Aspect 87. The milvexian for use of any one of the preceding aspects, wherein the administration results in minimal impairment of hemostasis in the human patient.
Aspect 88. The milvexian for use of any one of the preceding aspects, wherein the regimen comprises an immediate release tablet.
Aspect 89. The milvexian for use of aspect 88, wherein the immediate release tablet has a disintegration time in water of less than 20 seconds at 37° C
Aspect 90. The milvexian for use of any one of the preceding aspects, wherein the administration results in a milvexian plasma terminal half-life ranging from about 13 hours to about 16 hours.
Aspect 91. The milvexian for use of any one of the preceding aspects, wherein the regimen is administered without regard to the timing of food intake.
Aspect 92. The milvexian for use of any one of aspects 88-91, wherein the immediate release tablet is dispersed in an aqueous medium to form an aqueous dispersion.
Aspect 93. The milvexian for use of aspect 92, wherein the aqueous medium comprises water or apple sauce.
Aspect 94. The milvexian for use of any one of the preceding aspects, wherein the antiplatelet therapy comprises administering a single antiplatelet therapy (SAPT) for a period ranging from about 1 month to about 36 months.
Aspect 95. The milvexian for use of aspect 94, wherein the antiplatelet therapy comprises administering a SAPT for a period ranging from about 3 months to about 12 months.
Aspect 96. The milvexian for use of aspect 94, wherein the antiplatelet therapy comprises administering a SAPT for a period ranging from about 6 months to about 12 months.
Aspect 97. The milvexian for use of aspect 94, wherein the antiplatelet therapy comprises administering a SAPT for 3 months.
Aspect 98. The milvexian for use of aspect 94, wherein the antiplatelet therapy comprises administering a SAPT for 6 months.
Aspect 99. The milvexian for use of aspect 94, wherein the antiplatelet therapy comprises administering a SAPT for 12 months.
Aspect 100. The milvexian for use of aspect 94, wherein the antiplatelet therapy comprises administering a SAPT for 24 months.
Aspect 101. The milvexian for use of any one of aspects 94-100, wherein the SAPT is selected from aspirin, and a P2Y12 inhibitor.
Aspect 102. The milvexian for use of any one of aspects 94-101, wherein the SAPT comprises aspirin.
Aspect 103. The milvexian for use of aspect 102, wherein the aspirin is administered once daily.
Aspect 104. The milvexian for use of aspect 102 wherein the aspirin is administered once daily for a period of ranging from about 3 months to about 3 years.
Aspect 105. The milvexian for use of aspect 102, wherein the aspirin is administered once daily for 3 months.
Aspect 106. The milvexian for use of aspect 102, wherein the aspirin is administered once daily for 6 months.
Aspect 107. The milvexian for use of aspect 102, wherein the aspirin is administered once daily for 12 months.
Aspect 108. The milvexian for use of aspect 102, wherein the aspirin is administered once daily for 24 months.
Aspect 109. The milvexian for use of aspect 102, wherein the aspirin is administered once daily for 36 months.
Aspect 110. The milvexian for use of aspect 102, wherein the aspirin is administered twice daily.
Aspect 111. The milvexian for use of aspect 102, wherein the aspirin is administered once daily at an amount of 75 mg to 100 mg.
Aspect 112. The milvexian for use of aspect 102, wherein the aspirin is administered once daily at an amount of 75 mg to 100 mg for a period of 12 months.
Aspect 113. The milvexian for use of any one of aspects 94-101, wherein the SAPT comprises a P2Y12 inhibitor.
Aspect 114. The milvexian for use of any one of aspects 94-101, wherein the SAPT comprises a P2Y12 inhibitor selected from prasugrel, clopidogrel, selatogrel, or ticagrelor.
Aspect 115. The milvexian for use of aspect 113 or 114, wherein the P2Y12 inhibitor is administered for a period ranging from about 3 months to about 6 months.
Aspect 116. The milvexian for use of aspect 113 or 114, wherein the P2Y12 inhibitor comprises clopidogrel.
Aspect 117. The milvexian for use of aspect 113 or 114, wherein the P2Y12 inhibitor comprises ticagrelor.
Aspect 118. The milvexian for use of aspect 113 or 114, wherein the P2Y12 inhibitor comprises ticagrelor administered at 90 mg twice daily.
Aspect 119. The milvexian for use of aspect 113 or 114, wherein the P2Y12 inhibitor comprises ticagrelor administered at 90 mg twice daily for a period of 3 years.
Aspect 120. The milvexian for use of aspect 113 or 114, wherein the P2Y12 inhibitor comprises ticagrelor administered first followed by aspirin monotherapy administered at 20 mg twice daily.
Aspect 121. The milvexian for use of any one of aspects 113 or 114, wherein the SAPT comprises aspirin monotherapy administered first for a period of ranging from 30 days to 90 days followed by clopidogrel monotherapy.
Aspect 122. The milvexian for use of any one of aspects 113 or 114, wherein the SAPT comprises aspirin monotherapy administered first for a period of 60 days followed by clopidogrel monotherapy.
Aspect 123. The milvexian for use of any one of aspects 94-122, wherein the SAPT comprises aspirin monotherapy administered first for a period of 90 days followed by clopidogrel monotherapy.
Aspect 124. The milvexian for use of any one of aspects 94-122, wherein the SAPT comprises aspirin monotherapy administered first followed by clopidogrel monotherapy for a period of at least 2 months.
Aspect 125. The milvexian for use of any one of aspects 94-122, wherein the SAPT comprises aspirin monotherapy administered first for a period ranging from 30 days to 90 days followed by clopidogrel monotherapy for a period of at least 2 months.
Aspect 126. The milvexian for use of any one of aspects 1-93, wherein the antiplatelet therapy comprises a dual antiplatelet therapy (DAPT).
Aspect 127. The milvexian for use of aspect of 126, wherein the DAPT comprises aspirin and prasugrel.
Aspect 128. The milvexian for use of aspect of 126, wherein the DAPT comprises aspirin and prasugrel administered for 12 months.
Aspect 129. The milvexian for use of aspect of 126, wherein the DAPT comprises aspirin and clopidogrel.
Aspect 130. The milvexian for use of aspect of 126, wherein the DAPT comprises aspirin and clopidogrel administered for a period of 21 days to 12 months.
Aspect 131. The milvexian for use of aspect of 126, wherein the DAPT comprises aspirin and clopidogrel administered for a period of 21 days.
Aspect 132. The milvexian for use of aspect of 126, wherein the DAPT comprises aspirin and clopidogrel administered for a period of 6 months.
Aspect 133. The milvexian for use of aspect of 126, wherein the DAPT comprises aspirin and clopidogrel administered for a period of 12 months.
Aspect 134. The milvexian for use of aspect of 126, wherein the DAPT comprises aspirin and ticagrelor.
Aspect 135. The milvexian for use of aspect of 126, wherein the DAPT comprises aspirin and ticagrelor administered for 12 months.
Aspect 136. The milvexian for use of aspect 126, wherein the antiplatelet therapy comprises a DAPT consisting of aspirin administered once daily at 75 mg to 100 mg and a P2Y12 inhibitor.
Aspect 137. The milvexian for use of aspect 126, wherein the antiplatelet therapy comprises a DAPT administered first followed by administering a SAPT.
Aspect 138. The milvexian for use of aspect 126, wherein the antiplatelet therapy comprises a DAPT administered first followed by administering aspirin monotherapy.
Aspect 139. The milvexian for use of aspect 126, wherein the antiplatelet therapy comprises a DAPT consisting of aspirin 75-100 mg once daily and a P2Y12 inhibitor administered for a first period of 12 months followed by administering aspirin monotherapy for a second period of 6 months.
Aspect 140. The milvexian for use of aspect 126, wherein the antiplatelet therapy comprises a DAPT consisting of aspirin 75-100 mg once daily and a P2Y12 inhibitor administered for a first period of 21 days followed by administering aspirin monotherapy for a second period of 6 months.
Aspect 141. The milvexian for use of aspect 126, wherein the antiplatelet therapy comprises a DAPT consisting of aspirin 75-100 mg once daily and clopidogrel 75 mg once daily administered for a first period of 21 days followed by administering aspirin monotherapy for a second period of 6 months.
Aspect 142. The milvexian for use of aspect 126, wherein the antiplatelet therapy comprises a DAPT consisting of aspirin 75-100 mg once daily and a P2Y12 inhibitor administered for a first period of 21 days followed by administering aspirin monotherapy for a second period of 12 months.
Aspect 143. The milvexian for use of aspect 126, wherein the antiplatelet therapy comprises a DAPT consisting of aspirin 75-100 mg once daily and ticagrelor 90 mg twice daily.
Aspect 144. The milvexian for use of aspect 126, wherein the antiplatelet therapy comprises a DAPT consisting of aspirin 75-100 mg once daily and ticagrelor 90 mg twice daily administered for a period of 12 months.
Aspect 145. The milvexian for use of aspect 126, wherein the antiplatelet therapy comprises a DAPT consisting of aspirin 81-100 mg once daily and ticagrelor 90 mg twice daily.
Aspect 146. The milvexian for use of aspect 126, wherein the antiplatelet therapy comprises a DAPT consisting of aspirin 81-100 mg once daily and ticagrelor 90 mg twice daily administered for 3 years.
Aspect 147. The milvexian for use of aspect 126, wherein the antiplatelet therapy comprises a DAPT consisting of aspirin 75-100 mg once daily and ticagrelor 90 mg twice daily administered for a first period of 12 months followed by administering aspirin monotherapy 75-100 mg once daily for a second period of 12 months.
Aspect 148. The milvexian for use of aspect 126, wherein the antiplatelet therapy comprises a DAPT consisting of aspirin 75-100 mg once daily and ticagrelor 90 mg twice daily administered for a first period of 12 months followed by administering ticagrelor 90 mg twice daily for a second period of 23 months.
Aspect 149. The milvexian for use of any one of the previous aspects, wherein the clinical benefit of reducing the incidence rates of the ischemic stroke by the regimen is maintained throughout a treatment period of 90 days.
Aspect 150. The milvexian for use of any one of the previous aspects, wherein the clinical benefit of reducing the incidence rates of the ischemic stroke is maintained throughout a treatment period of 180 days.
Aspect 151. The milvexian for use of any one of the previous aspects, wherein the clinical benefit of reducing the incidence rates of the ischemic stroke is maintained throughout a treatment period of 12 months.
Aspect 152. The milvexian for use of any one of the preceding aspects, wherein administration of the regimen reduces the patient's FXI clotting activity by about 7% to about 20% relative to baseline.
Aspect 153. The milvexian for use of any one of the preceding aspects, wherein administration of the regimen results is a prolongation of activated partial thromboplastin time (aPTT) ranging from about 27% to about 64% relative to baseline.
Aspect 154. The milvexian for use of any one of the preceding aspects, where the administration does not result in a statistically significant increase in major bleeding complications.
Aspect 155. The milvexian for use of any one of the preceding aspects, wherein the pharmaceutical composition comprising milvexian (or pharmaceutically acceptable salt or solvate thereof) is a solid oral pharmaceutical composition.
Aspect 156. The milvexian for use of aspect 155, wherein the solid oral pharmaceutical composition is a tablet.
Aspect 157. The milvexian for use of aspect 156, wherein the tablet is an immediate release tablet.
Aspect 158. The milvexian for use of any one of the preceding aspects, wherein the human patient is unable to swallow a tablet dosage form.
Aspect 159. The milvexian for use of any one of aspects 156-158, wherein the tablet is dispersed in an aqueous medium to form an aqueous dispersion.
Aspect 160. The milvexian for use of aspect 159, wherein the aqueous medium is water, saline, phosphate buffer, vegetable juice, or fruit juice, including, for example, apple sauce.
Aspect 161. The milvexian for use of aspect 159 or aspect 160, wherein the aqueous dispersion is administered to the human patient via a nasogastric tube or spoon.
Aspect 162. The milvexian for use of any one of the preceding aspects, wherein the pharmaceutical composition is administered orally as an aqueous dispersion by dispersing the oral tablet in an aqueous medium in less than 1 minute.
Aspect 163. The milvexian for use of any one of the preceding aspects, wherein the pharmaceutical composition comprises an amorphous form of milvexian admixed with a polymer.
Aspect 164. The milvexian for use of any one of the preceding aspects, wherein the pharmaceutical composition comprises a spray dried amorphous solid dispersion consisting of 75% w/w milvexian and 25% w/w of HPMC-AS.
Aspect 165. The milvexian for use of any one of the preceding aspects, wherein the milvexian for use results in no clinically significant QTc interval prolongation.

### Additional Aspects

Aspect 1. A method of preventing adverse cerebrovascular events or adverse cardiovascular events in a human patient with acute coronary syndrome, wherein the method comprises administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combination thereof; wherein the pharmaceutical composition is administered twice daily.
Aspect 2. The method of aspect 1, wherein the antiplatelet therapy is a P2Y12 inhibitor.
Aspect 3. The method of aspect 2, wherein the P2Y12 inhibitor is clopidogrel, ticagrelor, or prasugrel.
Aspect 4. The method of any one of aspects 1-3, wherein the antiplatelet therapy is aspirin.
Aspect 5. The method of any one of aspects 1-4, wherein the human patient is treated with aspirin and clopidogrel combination therapy from day 1 to day 21, followed by aspirin monotherapy for at least 90 days.
Aspect 6. The method of aspect 5, wherein the patient is treated with aspirin and/or clopidogrel without milvexian dose adjustment.
Aspect 7. The method of any one of the preceding aspects, wherein the method is for primary prevention of adverse cerebrovascular events or adverse cardiovascular events.
Aspect 8. The method of any one of the preceding aspects, wherein the method is for secondary prevention of adverse cerebrovascular events or adverse cardiovascular events.
Aspect 9. The method of any one of aspects 1-8, wherein the adverse cerebrovascular events or adverse cardiovascular events comprises one or more of stroke, heart attack, or death.
Aspect 10. The method of any one of aspects 1-9, wherein the adverse cerebrovascular events or adverse cardiovascular events occur in an organ selected from heart, brain, limb, blood supply system, or vessel.
Aspect 11. The method of any one of aspects 1-10, wherein the adverse cerebrovascular events or adverse cardiovascular events comprise one or more major adverse cardiovascular event (MACE) selected from the group consisting of cardiovascular death, nonfatal myocardial infarction, ischemic stroke, and combinations thereof.
Aspect 12. The method of any one of aspects 1-11, wherein the adverse cerebrovascular events or adverse cardiovascular events comprise one or more major adverse vascular events (MAVE) selected from the group consisting of MACE; major adverse limb events (MALE) including one or more selected from acute limb ischemia, chronic limb ischemia, or major amputation; symptomatic venous thromboembolic events, and combinations thereof.
Aspect 13. The method of any one of aspects 1-12, wherein the adverse cerebrovascular events or adverse cardiovascular events are selected from the group consisting of arrhythmogenic cardiomyopathy (ACM), nonfatal myocardial infarction, ischemic stroke, and combinations thereof.
Aspect 14. The method of any one of aspects 1-11, wherein the adverse cerebrovascular events or adverse cardiovascular events comprises cardiovascular death.
Aspect 15. The method of any one of aspects 1-10, wherein the adverse cerebrovascular events or adverse cardiovascular events comprises arrhythmogenic cardiomyopathy (ACM).
Aspect 16. A method for preventing adverse cardiovascular events in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof,
   wherein the pharmaceutical composition is administered twice daily; and
   wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb inschemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; and transient ischemic attack (TIA).
Aspect 17. The method of aspect 16, wherein administration of the regimen results in a relative risk reduction (RRR) in the occurrence of symptomatic venous thromboembolism (VTE: (pulmonary embolism (PE), deep vein thrombosis (DVT) in the patient of at least 25%, without increased bleeding, versus a placebo group.
Aspect 18. The method of aspect 16, wherein the adverse cardiovascular event is one or more of CV death, MI, or ischemic stroke.
Aspect 19. A method for reducing incidence rate of the one or more of adverse thrombotic event selected from new ischemic stroke, MI, or all-cause death in a human patient diagnosed with acute coronary syndrome, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.
Aspect 20. The method of aspect 19, wherein administration of the regimen results in a relative risk is 0.85 or less relative to the placebo.
Aspect 21. A method for preventing ischemic stroke in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.
Aspect 22. The method of aspect 21, wherein administration of the regimen results in a relative risk reduction (RRR) in the occurrence of clinical ischemic stroke events in the patient is at least 25%, without increased bleeding, versus a placebo group.
Aspect 23. The method of aspect 21 or aspect 22, wherein the clinical benefit of reducing the incidence rates of the clinical ischemic stroke by the regimen is maintained throughout a treatment period of 90 days.
Aspect 24. The method of any one of the preceding aspects, wherein administration of the regimen reduces the patient's FXI clotting activity by about 7% to about 20% relative to baseline.
Aspect 25. The method of any one of the preceding aspects, wherein administration of the regimen results is a prolongation of activated partial thromboplastin time (aPTT) ranging from about 27% to about 64% relative to baseline.
Aspect 26. The method of any one of the preceding aspects, where the administration does not result in a statistically significant increase in major bleeding complications.
Aspect 27. The method of any one of the preceding aspects, wherein the pharmaceutical composition comprising milvexian (or pharmaceutically acceptable salt or solvate thereof) is a solid oral pharmaceutical composition.
Aspect 28. The method of aspect 27, wherein the solid oral pharmaceutical composition is a tablet.
Aspect 29. The method of aspect 28, wherein the tablet is an immediate release tablet.
Aspect 30. The method of aspect 28 or aspect 29, wherein the tablet has a disintegration time in water of less than 20 seconds.
Aspect 31. The method of any one of the preceding aspects, wherein the administration results in a milvexian plasma half-life ranging from about 13 hours to about 16 hours.
Aspect 32. The method of any one of the preceding aspects, wherein the administration results in milvexian plasma concentration reaching steady-state at about 3 days to 6 days.
Aspect 33. The method of any one of the preceding aspects, wherein the pharmaceutical composition comprising milvexian (or pharmaceutically acceptable salt or solvate thereof) is administered without regard to the timing of food intake.
Aspect 34. The method of any one of the preceding aspects, wherein the human patient is unable to swallow a tablet dosage form.
Aspect 35. The method of any one of aspects 28-34, wherein the tablet is dispersed in an aqueous medium to form an aqueous dispersion.
Aspect 36. The method of aspect 35, wherein the aqueous medium is water, saline, phosphate buffer, vegetable juice, or fruit juice, including, for example, apple sauce.
Aspect 37. The method of aspect 35 or aspect 36, wherein the aqueous dispersion is administered to the human patient via a nasogastric tube or spoon.
Aspect 38. The method of aspect any one of aspects 28-37, wherein the pharmaceutical composition is administered orally as an aqueous dispersion by dispersing the oral tablet in an aqueous medium in less than 1 minute.

Those skilled in the art will recognize, or be able to ascertain using no more than routine experimentation, many equivalents to the embodiments described herein.

All publications, patents and patent applications mentioned in this specification are herein incorporated by reference into the specification to the same extent as if each individual publication, patent or patent application was specifically and individually indicated to be incorporated herein by reference.

### EXAMPLES

### Example 1. a Phase 3, Randomized, Double-Blind, Placebo-Controlled, Event-Driven Study to Demonstrate the Efficacy and Safety of Milvexian, an Oral Factor XIa Inhibitor, after a Recent Acute Coronary Syndrome

This is a Phase 3 multicenter, randomized, double-blind, placebo-controlled, parallel-group, event driven, superiority, group-sequential study to evaluate the efficacy and safety of milvexian in participants enrolled within 7 days of an ACS (defined as STEMI or NSTEMI or UA with biomarker positivity), who have undergone cardiac catheterization with PCI or who are being managed conservatively with or without catheterization, and who are receiving antiplatelet therapy standard-of-care as determined by the investigator.

### Study Population

Approximately 16,000 participants are randomized within 7 days of an ACS event to reach the prespecified number of 875 participants with one or more primary efficacy outcome events.

Inclusion Criteria: Each potential participant must satisfy all of the following criteria to be enrolled in the study:

### Age

1. ≥18 years of age.

### Type of Participant and Disease Characteristics

2. Participants must have an index event that meets all 3 of the following criteria within 7 days prior to randomization:
   a) Clinical syndrome consistent with spontaneous cardiac ischemia
   b) Diagnosis of ACS (i.e., STEMI, non-STEMI, or UA)
   c) Cardiac biomarker elevation (e.g., troponin I, troponin T, CK-MB) above the upper limit of normal as determined by the local laboratory.
3. Participants must have at least 2 of the following risk factors:|
   a) Age 65 or older
   b) Diabetes mellitus
   c) History of a prior MI (other than index ACS event)
   d) Multivessel CAD (Note: this may be a history of multivessel CAD or a history of single vessel CAD with the index ACS event being in a different coronary artery, establishing multivessel CAD.)
   e) History of CABG surgery prior to index ACS event
   f) History of PAD or cerebrovascular disease (eg., carotid atherosclerosis, intracranial artery stenosis. Please note that participants with a history of TIA or stroke are excluded from enrollment.)
   g) Conservative management (i.e., no PCI or CABG after index ACS event)
   h) Any one or more of the following high-risk angiographic features
      a. Total stent length of >30 mm
      b. Thrombotic target lesion
      c. Bifurcation lesion treated with more than one stent
      d. Calcified target lesion treated with atherectomy
      e. Treatment of obstructive left main or proximal left anterior descending artery for index ACS (or clinical diagnosis of an anterior STEMI)

### Weight

Not applicable.

### Sex and Contraceptive/Barrier Requirements

4. All female participants of childbearing potential must have a negative highly sensitive serum β-hCG or urine test at screening.
5. A female participant must not be pregnant, breastfeeding, or planning to become pregnant until 4 days (5 half-lives) after the last dose of study intervention.
6. A female participant must be
   a. Not of childbearing potential
      b. Of childbearing potential and practicing a highly effective method of contraception (failure rate of <1% per year when used consistently and correctly) and agrees to remain on a highly effective method until 4 days after last dose (5 half-lives) after the last dose of study intervention—the end of relevant exposure.
7. A female participant using hormonal contraceptives should use an additional nonhormonal contraceptive method (above that required in Inclusion Criterion 6b) until 4 days after the last dose (5 half-lives) of study intervention—the end of relevant exposure.

Exclusion Criteria: Any potential participant who meets any of the following criteria will be excluded from participating in the study:

### Medical Conditions

1. MI secondary to ischemia due to either increased oxygen demand or decreased supply (Type 2 MI) or periprocedural MI as the index ACS event.
2. Minimal or no obstructive CAD on angiography performed for the index ACS event prior to any PCI (i.e., <50% stenosis visually as determined by the investigator).
3. Planned CABG or staged PCI after randomization (Note: participants may be evaluated for enrollment after completion of a planned staged PCI).
4. Any condition that requires chronic anticoagulation at the discretion of the investigator and/or local guidelines. Note: Participants with an indication for chronic antiplatelet therapy after placement of a bio-prosthetic non-mechanical valve (eg, transcatheter aortic valve replacement) do not satisfy this exclusion criterion and are eligible for study participation.
5. Conditions with a significant increased risk of bleeding (e.g., clinically significant bleeding within previous 3 months, known bleeding diathesis, etc.)
6. Requires dialysis on a permanent basis or has an eGFR<15 mL/min/1.73 m² at screening.
7. Current active liver disease (eg, acute hepatitis and known cirrhosis), including participants receiving antiviral treatment for hepatitis
8. Active cancer undergoing chemotherapy or radiation or treatment with immunotherapy
9. CABG performed during index event
10. History of ischemic stroke or TIA
11. Killip Class 3 or 4 at the time of randomization
12. History of any significant drug allergy (such as anaphylaxis, Stevens-Johnson Syndrome, toxic epidermal necrolysis, DRESS)
13. Known allergies, hypersensitivity, or intolerance to milvexian or its excipients (refer to the milvexian IB)
14. Known aPTT prolongation >1.5×the ULN or known congenital FXI deficiency

### Prior/Concomitant Therapy

15. Planned use of any disallowed therapies, ConcomitantTherapy, including isoniazid (INH)

### Prior/Concurrent Clinical Study Experience

16. Received an investigational study intervention or used an invasive investigational medical device within 4 weeks before the planned first dose of study intervention or is currently enrolled in an investigational study

### Diagnostic Assessments

17. Any of the following laboratory results, based on local laboratory, meeting thecriteria specified below prior to randomization, confirmed by repeat:
   Platelet count <75,000/L
   ALT>3×ULN
   Total bilirubin ≥1.5×ULN unless an alternative causative factor such as Gilbert's Syndrome is identified
   Hemoglobin <8.0 g/dL

### Other Exclusions

18. Employee of the investigator or study site, with direct involvement in the proposed study or other studies under the direction of that investigator or study site, as well as family members of the employees or the investigator
19. Any condition for which, in the opinion of the investigator, participation would not be in the best interest of the participant (eg, compromise the well-being) or that could prevent, limit, or confound the protocol-specified assessments or has a life expectancy of <12 months
20. At the time of screening, any participant who will not consider following the study contact schedule, or not allow a contact to the participant, or to the designated family members or health care practitioner, to determine any endpoint events and/or vital status, up until the end of the study should they prematurely discontinue study medication or withdraw from study participation.
21. Mental condition or cognitive impairment/dementia that makes the participant unable to understand the nature, potential risks and benefits of the study.
22. Participants who are incarcerated, including prisoners or participants compulsorily detained for treatment of psychiatric disease.
23. Known current substance abuse.

### Study Treatment and Visits

Participants receive either milvexian (25 mg twice daily orally) or a matching placebo on a background of standard-of-care antiplatelet therapy. Randomization is stratified by the intended, investigator-determined antiplatelet strategy into either 1) DAPT for >90 days (with or without de-escalation to SAPT), 2) DAPT for ≤90 days with de-escalation to SAPT, or 3) SAPT. The total study duration is approximately 3.5 years under current protocol assumptions.

### Background Antiplatelet Therapy:

Planned use SAPT or DAPT and duration of DAPT will be at the discretion of the Investigator, based on standard-of-care guidelines and will be recorded at randomization.

SAPT can be low-dose aspirin (≤100 mg per day) or a P2Y12 inhibitor.

P2Y12 inhibitor (alone or in combination with aspirin) can be clopidogrel, ticagrelor or prasugrel.

P2Y12 inhibitor may be changed or stopped. Such changes must be recorded in the eCRF.

De-escalation from DAPT to SAPT can drop either aspirin or P2Y12 inhibitor and must be recorded in the eCRF.

For example, background antiplatelet therapy regimen and duration may consist of: SAPT comprising ≤ASA 100 mg daily until end of study; SAPT comprising clopidogrel 75 mg daily until end of study; DAPT comprising ≤ASA 100 mg daily plus clopidogrel 75 mg daily for 90 days followed by clopidogrel 75 mg daily until the end of study; or all of the foregoing.

This study will include 3 phases: a screening phase up to 7 days after the index ACS event and prior to randomization, a double-blind treatment phase, and a 30-day follow-up phase. Treatment will begin on the day of randomization and continue until the target number of participants with one or more primary efficacy outcome events has been achieved (followed by an EOT visit [within 30 days after GTED]). Thus, treatment duration with study intervention will be variable depending on when the participant is randomized. It is anticipated that the last participant randomized will have a minimum of approximately 3 months of treatment with study intervention by the time of the GTED.

Study intervention may be taken without regard to the timing of food intake. For participants who are unable to swallow medication, study intervention can be dissolved in water and given via a nasogastric tube or in applesauce.

The milvexian dosage form is a film-coated, direct-compression tablet having the formulation described in Table 1 below:

TABLE 1
Film-coated Tablet of Milvexian at 100 mg Strength

| | Amount | | |
|---|---|---|---|
| | | Amount | Amount |
| | | per dose | per dose |
| Component | wt. % | (25 mg, Ex.17) | (100 mg, Ex.18) |
| SDP^{b} | 22.22 | 33.33 | 133.3 |
| Silicified Microcrystalline | 43.07 | 64.6 | 258.4 |
| cellulose | | | |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| OpadryQX 321A220063 | 3.0 | 4.5 | 18.0 |
| Yellow^{c} | | | |
| | | 154.5 | 618 |
| Tablet hardness | | Average 60N to | Average 140N to |
| | | 120N (Ph. | 220N (Ph. |
| | | Eur.2.9.8), | Eur.2.9.8), |
| | | preferably 90N | preferably 180N |
| Tablet friability | | <0.5% | <0.5% |
| Disintegration time in | | 13 | 18 |
| water (seconds) | | | |

a. The film-coated, direct-compression immediate release tablet of milvexian was prepared according to provisional application US 63/483,486, which is incorporated herein by its entirety.
^{b}SDP means spray dried power comprising spray-dried amorphous solid dispersion of milvexian in HPMC-AS-MG grade polymer in a weight ratio of 3:1 (milvexian: HPMC-AS-MG).
^{c}OpadryQX 321A220063 Yellow is composed of Macrogol (PEG) polyvinyl alcohol grafted copolymer, talc, titanium dioxide, glycerol monocaprylocaprate Type I, polyvinyl alcohol and iron oxide yellow.

As used herein, the term “disintegration time” refers to the time required for the tablet to break into particles under a given set of conditions. In some embodiments, the disintegration time is determined using the apparatus described in Eur. Ph. (PTZ-E Pharma Test, Hainburg, Germany), in distilled water at 37° C using disks.

The SDP (spray-dried powder) in the tablets (Ex. 17, Ex. 18) is prepared as follows. A solution containing about 12.3 wt. % of milvexian P1.Acetone (equivalent to about 11.25 wt. % of milvexian free form) and about 3.75 wt. % of HPMC-AS MG (AQOAT® AS-MG, Shin-Etsu Chemical Co., Ltd. (Niigata, Japan)) in a solvent mixture containing 80/20 w/w % DCM/MeOH is prepared. The clear solution at 21° C is spray dried using a Buchi B-290 spray dryer with a 35 Kg/hr drying gas flow-rate capacity, set of the following parameters: Atomization gas flow rate at 25 mm (301 L/hr); feed rate at 7.7 g/min; inlet/outlet temperatures at 67/44° C, condenser temperature of −19° C, spray nozzle orifice diameter of 0.7 mm, and spray nozzle cap diameter of 1.4 mm. The spray drying process proceeds for 11 min to give 11.5 g (89% yield) of wet SDP. The wet SDP is subject to drying for 24 hours in a vacuum oven (Heraeus, Model VT6130 M) at 40° C, with nitrogen flow, and a vacuum of approximately 200 mbar to give 10.7 g (83% yield) of desired dry SDP.

The SDP product is a white powder having an assay of 98.8% and a purity of 99.9% by HPLC. The PXRD diffraction pattern shows a halo pattern with no crystalline peaks indicating the product is amorphous.

### Efficacy Evaluations

The primary, secondary and exploratory efficacy endpoint events encompass the following individual events, often grouped in composites in various combinations, including ACM; CV death; MI; UA; “all stroke” or “any stroke” (ischemic, hemorrhagic, or unknown cause); ischemic stroke; ALI; major vascular (non-traumatic) limb amputation; symptomatic VTE (PE, DVT); ischemia-driven coronary revascularization; stent thrombosis; hospitalization for any reason, categorized as (1) planned or unplanned, (2) for arterial or venous thrombotic event or neither; TIA. An independent CEC will adjudicate efficacy outcome events as defined in the CEC charter (Table 2).

TABLE 2
Primary and Secondary Endpoints

| Objective | Endpoint |
|---|---|
| Primary | |
| To demonstrate that milvexian is superior to | Time to the first |
| placebo, in addition to standard-of-care, in | occurrence of MACE |
| reducing the risk of MACE (the composite of | |
| CV death, MI, and ischemic stroke) | |
| Secondary | |
| To demonstrate that milvexian is superior to | Time to the first |
| placebo, in addition to standard-of-care, in | occurrence of MAVE |
| reducing the risk of MAVE (the composite of | |
| CV death, MI, ischemic stroke, MALE, and | |
| symptomatic VTE) | |
| To determine whether milvexian reduces the | Time to the first |
| risk of the composite of ACM, MI, and | occurrence of ACM, |
| ischemic stroke compared to placebo | MI, and ischemic stroke |
| To determine whether milvexian reduces the | Time to CV death |
| risk of CV death compared to placebo | |
| To determine whether milvexian reduces the | Time to ACM |
| risk of ACM compared to placebo | |

### Pharmacokinetic Evaluations

Plasma samples are collected from approximately 5,000 participants. The concentration of milvexian is measured in samples collected from participants who were treated with milvexian using a validated, specific, and sensitive (e.g., LC-MS/MS) method by or under the supervision of the sponsor.

Residual plasma PK samples may be stored for future analysis and for metabolite profiling. Based on the individual plasma concentration-time data and using the actual dose and sampling times, PK parameters (e.g., apparent clearance, etc.) for exposure-response analysis of milvexian and associated variables are derived using population PK modeling.

### Pharmacodynamic and Biomarker Evaluations

Plasma samples for PD and biomarker assays are collected in approximately 1,000 participants in the study. The PD assay is aPTT. Exploratory biomarkers of disease pathophysiology are proteomics and D-dimer.

### Safety Evaluations

The bleeding endpoints encompass the following individual events, often grouped in composites in various combinations, including BARC categories 2, 3a, 3b, 3c, 4 and 5; ISTH categories major and CRNM; GUSTO categories (1) severe or life threatening and (2) moderate; and TIMI categories non-CABG-related major, CABG-related major, minor, and requiring medical attention. These bleeding outcome events are adjudicated by an independent CEC as defined in the CEC charter. Overall safety and tolerability are assessed via evaluation of AEs, clinical laboratory tests, and vital signs.

### Statistical Methods

Summaries by treatment group using appropriate descriptive statistics are provided for all study variables including demographic and baseline characteristics. No imputation is applied, unless specified otherwise in the SAP. Descriptive statistics such as mean, median, standard deviation, minimum, and maximum are used to summarize continuous variables. Counts and percentages are used to summarize categorical variables. The Kaplan-Meier method is used to summarize time-to-event variables. Graphical data displays may also be used to summarize the data.

Unless stated otherwise, all statistical tests are interpreted at a nominal (that is, without adjustment for multiplicity) 2-sided significance level of 0.05 and all CIs at a nominal 2-sided level of 95%.

The stratified log-rank test is used for testing the primary hypothesis. To control the family-wise type I error rate at alpha of 0.05 (2-sided) in testing for primary and secondary efficacy outcomes, if superiority of milvexian over placebo on the primary efficacy outcome is established, superiority of milvexian over placebo on secondary efficacy outcomes is tested sequentially using a closed testing procedure in the predefined hierarchical order.

The stratified Cox regression is used to estimate the HR and 95% CIs for the treatment effect on the primary efficacy endpoint.

An interim analysis for futility is planned when approximately 60% of target number of participants with the primary efficacy events are observed. As the study will not stop for superiority, the type I error rate does not need to be adjusted.

### Example 2. Prevention of New Ischemic Stroke or New Covert Brain Infarction Following Acute Ischemic Stroke or Transient Ischemic Attack

The AXIOMATIC-SSP Antithrombotic treatment with Factor Xia inhibition to Optimize Management of Acute Thromboembolic events in Secondary Stroke Prevention) study was a multi-center, Phase 2, randomized, double-blind, placebo-controlled, dose-ranging study of milvexian for the prevention of new ischemic stroke or new covert brain infarction in participants receiving ASA and clopidogrel following an acute ischemic stroke or TIA. The primary objective was to estimate the dose-response relationship of milvexian in participants with ischemic stroke or TIA treated with ASA and clopidogrel by assessing the composite of new ischemic stroke during the treatment period and new covert brain infarction detected by magnetic resonance imaging (MRI) at Day 90 (assessed by central review). The target randomization ratio by the end of the study was 2:1:1:1:1:1 (placebo, milvexian 25 mg QD, 25 mg BID, 50 mg BID, 100 mg BID, and 200 mg BID). Two additional dosing arms, milvexian 50 mg QD and 100 mg QD were included in the original design, but the protocol was amended (revised protocol 05) to close randomization to these arms to simplify the study design while maintaining scientific rigor to attain trial objectives, minimize the number of study participants exposed to the new investigational drug, reduce the burden on participants and study centers, and streamline study procedures. Due to the early termination of enrollment in the 50 mg QD and 100 mg QD treatment arms and the limited number of participants in both groups, these arms are not included in the presented analyses for efficacy, but are included in baseline characteristics (combined milvexian), subject disposition, and safety.

The study enrolled participants ≥40 years of age who had ischemic stroke or TIA. Ischemic stroke was defined as a neurological deficit attributable to a non-lacunar, acute brain infarction detected by neuroimaging and relevant to the clinical symptoms and National Institutes of Health Stroke Score (NIHSS)≤7 at the time of randomization. TIA was defined as acute onset neurological deficit attributable to focal ischemia of the brain by history or examination, with complete resolution of the deficit and no brain infarction on neuroimaging and ABCD score ≥6 or presence of motor symptoms. Evidence of relevant intracranial or cervical arterial atherosclerotic plaque, ulceration or thrombus in a feeding artery documented by imaging was required regardless of whether the index event was ischemic stroke or TIA, and all participants were required to have a Modified Rankin Score ≤3 before the index event. Eligible participants were screened for potential inclusion into the study as soon as possible after presentation and randomized within 48 hours of the index event. Following informed consent, participants received ASA 100 mg QD and a single loading dose of clopidogrel 300 mg if not already given as standard of care. Participants were then randomized to receive milvexian or placebo, plus open-label uncoated ASA 100 mg in combination with clopidogrel 75 mg QD for the next 21 days. Participants continued treatment from Day 22 through Day 90 with milvexian or placebo, plus open-label 100 mg uncoated ASA only. A baseline MRI was performed within 48 hours of the onset of the index event and prior to randomization. A second MRI was performed on Day 90±6 days. All participants were required to remain on study treatment until the Day 90 MRI was performed up to Day 96.

From 27 Jan. 2019 to 24 Dec. 2021, 2,799 participants were enrolled and 2,366 participants were randomized into 8 treatment arms. The 200 mg BID arm was opened on 5 Jun. 2021 after approximately 1,387 participants were enrolled and after the DMC reviewed unblinded data to assess the safety of opening the dose arm. The randomization of participants to the 50 mg QD (N=22) and 100 mg QD (N=18) treatment arms was terminated with the implementation of revised protocol amendment 05 as previously described. Overall, the mean (standard deviation [SD]) age of participants was 69.6 (±10.81) years. The majority of the participants was male and white. Overall, most index events (75.7%) were ischemic stroke, with an NIHSS ≤5 in approximately 96% of randomized participants. TIA represented 24.1% of index events; 53.4% of the TIA participants had an ABCD2 score of >6. The baseline disease characteristics were balanced among the treatment groups, with the exception of NIHSS score 6 to 7 in the 200 mg BID arm (4.7%) which was higher than in the other groups (range: 2.1% to 4.0%). More participants with NIHSS score 6 or 7 were assigned to the highest dose group due to the staggered opening of that treatment arm and broadening the NIHSS inclusion from ≤5 to ≤7 which occurred with the same protocol amendment. The most common comorbidities/risk factors were hypertension, diabetes, history of cigarette smoking, and hypercholesterolemia. All baseline conditions were well balanced across treatment arms.

The dose-response relationship between milvexian treatment (including placebo) for the primary composite endpoint of symptomatic ischemic stroke during the study period and new covert brain infarction on MRI at Day 90 was analyzed based on a generalized Multiple Comparison Procedure-Modelling (MCP-Mod) analysis, and results were presented by treatment arm.

In the AXIOMATIC-TKR study, the milvexian dose of 25 mg twice daily showed comparable efficacy to enoxaparin with a favorable safety profile. In the AXIOMATIC-SSP study the 25 mg twice daily milvexian dose was numerically better than placebo for preventing ischemic strokes (HR 0.69, 95% CI 0.36-1.30) with no further improvements in efficacy observed at the higher doses.

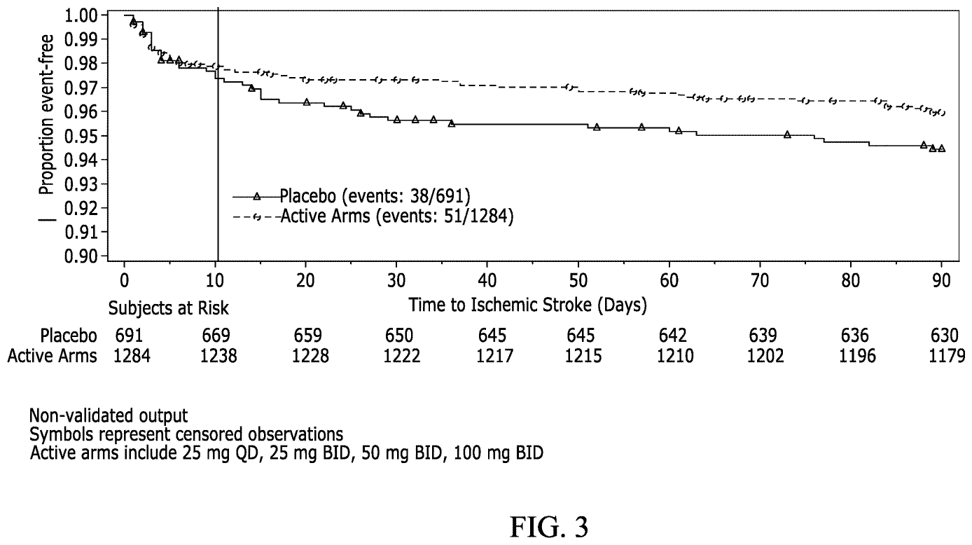

The primary endpoint was numerically lower at the 50 mg and 100 mg BD doses but there was no trend in dose-response. Milvexian numerically reduced the incident rates of ischemic stroke events (acute IS and excluding covert brain infarction) in the ITT population at all milvexian doses except 200 mg twice daily (see Table 3 below). Milvexian is effective at reducing the ischemic strokes in patients after an ischemic stroke or TIA at doses from 25 mg BID to 100 mg BID and these doses demonstrated about 30% relative risk reduction (RRR) in the occurrence of ischemic stroke in the treatment groups versus the placebo groups (See FIG. 3).

TABLE 3
Summary of Ischemic Stroke and
Undetermined Stroke-All Randomized
Participants

| | Placebo | 25 QD | 25 BID | 50 BID | 100 BID | 200 BID |
|---|---|---|---|---|---|---|
| | N = | N = | N = | N = | N = | N = |
| | 691 | 328 | 318 | 328 | 310 | 351 |
| Symptomatic | | | | | | |
| Stroke | | | | | | |
| Events | | | | | | |
| Subjects | 38 (5.5) | 15 (4.6) | 12 (3.8) | 13 (4.0) | 11 (3.5) | 27 (7.7) |
| With Event n | | | | | | |
| Incidence | | | | | | |
| Rate (n/N %) | | | | | | |
| Subjects | 38 (5.5) | 15 (4.6) | 12 (3.8) | 13 (4.0) | 11 (3.5) | 27 (7.7) |
| With | | | | | | |
| Ischemic | | | | | | |
| Stroke n | | | | | | |
| Incidence | | | | | | |
| Rate (n/N %) | | | | | | |
| Subjects | 0 | 0 | 0 | 0 | 0 | 0 |
| With Un- | (0) | (0) | (0) | (0) | (0) | (0) |
| determined | | | | | | |
| Stroke n | | | | | | |
| Incidence | | | | | | |
| Rate (n/N %) | | | | | | |
| Subjects | 0 | 0 | 0 | 0 | 0 | 0 |
| With | (0) | (0) | (0) | (0) | (0) | (0) |
| Hemorragic | | | | | | |
| Stroke n | | | | | | |
| Incidence | | | | | | |
| Rate (n/N %) | | | | | | |
| (95% CI) (1) | (3.8, | (2.3, | (1.7, | (1.9, | (1.5, | (4.9, |
| | 7.2) | 6.8) | 5.9) | 6.1) | 5.6) | 10.5) |
| Relative | | 0.83 | 0.69 | 0.72 | 0.65 | 1.40 |
| Risk (RR) | | | | | | |
| 95% CI for | | (0.46, | (0.36, | (0.39, | (0.33, | (0.87, |
| RR (2) | | 1.49) | 1.30) | 1.33) | 1.25) | 2.25) |

Clinical events are included up to Day 21
(1) Wald 95% CI within group
(2) 95% CIs for RR are constructed using Wald Confidence Limits
Program Source: BMS_GBS\CV010\OZA75318\Biostatistics\Production\Tables\CSR\rt-ef-clinstk.sas

A sub-group analysis of symptomatic ischemic stroke by stroke-subtype, confirmed that the majority of new ischemic stroke events were due to large vessel atherosclerosis across all milvexian treatment arms and placebo.

The rate of the secondary efficacy endpoint composite event of new ischemic stroke, MI, and all-cause death was lower in milvexian than placebo at dose levels of 25 mg QD to 100 mg BID (Table 4). The relative risk of this composite event in milvexian compared with placebo ranged from 0.78 to 0.85 over the 25 mg QD to 100 mg BID range, but there was no apparent dose-response trend.

TABLE 4
Summary of Event Rate of Composite of New Ischemic
Stroke, MI and All Cause
Death-All Randomized Subjects

| | Placebo | 25 QD | 25 BID | 50 BID | 100 BID | 200 BID |
|---|---|---|---|---|---|---|
| | N = | N = | N = | N = | N = | N = |
| | 691 | 328 | 318 | 328 | 310 | 351 |
| New | | | | | | |
| Ischemic | | | | | | |
| Stroke, MI | | | | | | |
| and All | | | | | | |
| Cause | | | | | | |
| Death n | | | | | | |
| (n/N %) | | | | | | |
| Subjects | 42 (6.1) | 17 (5.2) | 15 (4.7) | 16 (4.9) | 16 (5.2) | 33 (9.4) |
| With Event | | | | | | |
| n | | | | | | |
| (n/N%) | | | | | | |
| Ischemic | 38 (5.5) | 15 (4.6) | 12 (3.8) | 13 (4.0) | 11 (3.5) | 27 (7.7) |
| Stroke n | | | | | | |
| (n/N%) | | | | | | |
| Un - | 0 (0) | 0 (0) | 0 (0) | 0 (0) | 0 (0) | 0 (0) |
| determined | | | | | | |
| Stroke n | | | | | | |
| (n/N%) | | | | | | |
| Myocardial | 2 (0.3) | 1 (0.3) | 2 (0.6) | 1 (0.3) | 2 (0.6) | 3 (0.9) |
| Infarction n | | | | | | |
| (n/N%) | | | | | | |
| All Cause | 5 (0.7) | 3 (0.9) | 3 (0.9) | 2 (0.6) | 5 (1.6) | 4 (1.1) |
| Death n | | | | | | |
| (n/N%) | | | | | | |
| (95% CI) | (4.3, | (2.8, | (2,4, | (2.5, | (2.7, | (6.3, |
| (1) | 7.9) | 7.6) | 7.0) | 7.2) | 7.6) | 12.5) |
| Relative | | 0.85 | 0.78 | 0.80 | 0.85 | 1.55 |
| Risk (RR) | | | | | | |
| vs placebo | | | | | | |
| (2) | | | | | | |
| 95% CI | | (0.49, | (0.44, | (0.46, | (0.49, | (1.0, |
| for RR vs | | 1.47) | 1.38) | 1.41) | 1.49) | 2.4) |
| placebo (2) | | | | | | |

Clinical events and deaths are included up to Day 90. The randomization of subjects to the 50 mg QD (N = 22) and 100 mg QD (N = 18) treatment arms was terminated with the implementation of a revised protocol.
(1) Wald 95% CI within group
(2) 95% CIs for RR are constructed using Wald Confidence Limits

Rates of participants completing the treatment period were similar between the placebo and milvexian treatment arms over the dose range of 25 mg QD to 100 mg BID. 20.700 to 25.8% of participants discontinued treatment early over the milvexian dose range of 25 mg QD to 100 mg BID, compared with 23.4% of participants in the placebo arm. The 200 mg BID arm had a higher rate of early treatment discontinuation (31.60%) primarily due to AEs. Similarly, the overall most common reason for participants not completing the study treatment period in the other dose arms was AEs.

SAS® version 9 or higher was used for statistical analyses, tabulations, and graphical presentations. The DoseFinding package for R (version 3.1.3 or higher) was also used in the conduct of the multiple comparisons modelling analyses.

A subset of the ITT Population that includes subjects with at least one PD endpoint (total 1995 subjects) assessed following the first dose of milvexian. Percent (%) change from baseline for aPTT and Factor XI clotting activity are summarized by treatment group and nominal time points. In addition, Exposure-Response relationship (E-R) analyses was conducted to explore the relationship of milvexian exposure to the percent (%) change from baseline in aPTT and Factor XI clotting activity using data collected from all treated subjects (total 2334 subjects). Dose-dependent increases in aPTT and dose-dependent decreases in the Factor XI clotting activity were observed for the milvexian treated groups.

The summary statistics for Activated Partial Thromboplastin Time (aPTT) and percent change from baseline for the pharmacodynamic population is as in the Table 5 below:

TABLE 5
Summary Statistics for Activated Partial Thromboplastin Time (aPTT) and
percent change from baseline for the pharmacodynamic population

| Visit | Lab Test: aPTT (seconds) | | | | | | | | | | |
|---|---|---|---|---|---|---|---|---|---|---|---|
| Time Point | Value | | | | | | Percent Change from Baseline | | | | |
| Treatment Group | N | Mean | SE | Min | Median | Max | Mean | SE | Min | Median | Max |

BASELINE Pre-dose

| | | | | | | |
|---|---|---|---|---|---|---|
| placebo | 645 | 26.46 | 0.14 | 14.7 | 25.90 | 55.9 |
| 25 mg BID | 284 | 27.19 | 0.36 | 19.1 | 26.25 | 105.7 |

DAY 1 4 h

| | | | | | | | | | | | |
|---|---|---|---|---|---|---|---|---|---|---|---|
| placebo | 575 | 27.56 | 0.37 | 18.7 | 25.90 | 121.4 | 5.18 | 1.35 | −55.3 | 0.4 | 383.7 |
| 25 mg BID | 260 | 35.06 | 0.5 | 19.9 | 34.65 | 92.1 | 31.05 | 1.79 | −34.4 | 27.06 | 121.4 |

DAY1 12 h

| | | | | | | | | | | | |
|---|---|---|---|---|---|---|---|---|---|---|---|
| placebo | 570 | 27.20 | 0.56 | 14.6 | 25.90 | 319.5 | 3.21 | 1.67 | −55.8 | −0.17 | 839.7 |
| 25 mg BID | 246 | 35.27 | 0.40 | 23.8 | 34.30 | 64.7 | 32.67 | 1.57 | −37.2 | 30.41 | 141.2 |

DAY 1 24 h

| | | | | | | | | | | | |
|---|---|---|---|---|---|---|---|---|---|---|---|
| placebo | 582 | 26.95 | 0.48 | 14.7 | 25.75 | 251.9 | 3.35 | 1.91 | −55.5 | −1.22 | 847.0 |
| 25 mg BID | 259 | 39.68 | 0.59 | 25.7 | 38.60 | 121.3 | 49.41 | 2.40 | −67.5 | 44.40 | 354.3 |

DAY 21 anytime

| | | | | | | | | | | | |
|---|---|---|---|---|---|---|---|---|---|---|---|
| placebo | 559 | 25.98 | 0.14 | 14.7 | 25.50 | 46.9 | -0.78 | 0.59 | −56.1 | −1.27 | 97.4 |
| 25 mg BID | 237 | 45.20 | 0.79 | 25.9 | 43.80 | 138.1 | 69.60 | 3.28 | −66.7 | 64.26 | 425.1 |

DAY 90 anytime

| | | | | | | | | | | | |
|---|---|---|---|---|---|---|---|---|---|---|---|
| placebo | 503 | 26.77 | 0.19 | 15.2 | 26.00 | 74.0 | 2.47 | 0.77 | −54.0 | 0.83 | 184.6 |
| 25 mg BID | 226 | 42.15 | 0.73 | 23.2 | 41.85 | 125.3 | 58.30 | 3.20 | −55.5 | 54.37 | 452.0 |

The FXI clotting activity observed in this study was as follows:

TABLE 6
Summary Statistics for Factor XI Clotting Activity Measurements and
Percent Change from Baseline-Pharmacodynamic Population

| Visit | Lab Test: FXI Clotting Activity (%) | | | | | | | | | | |
|---|---|---|---|---|---|---|---|---|---|---|---|
| Time Point | Value | | | | | | Percent Change | | | | |
| Treatment Group | N | Mean | SE | Min | Median | Max | Mean | SE | Min | Median | Max |

BASELINE Pre-dose

| | | | | | | |
|---|---|---|---|---|---|---|
| placebo | 645 | 113.0 | 0.93 | 10.0 | 113.0 | 150.0 |
| 25 mg BID | 281 | 112.9 | 1.27 | 37.0 | 112.0 | 150.0 |

DAY 1 4 h

| | | | | | | | | | | | |
|---|---|---|---|---|---|---|---|---|---|---|---|
| placebo | 569 | 111.6 | 1.00 | 10.0 | 113.0 | 150.0 | 2.19 | 2.78 | −89.5 | −0.93 | 1150 |
| 25 mg BID | 254 | 103.3 | 1.43 | 10.0 | 100.0 | 150.0 | −7.21 | 1.24 | −89.5 | −8.48 | 186.5 |

DAY1 12 h

| | | | | | | | | | | | |
|---|---|---|---|---|---|---|---|---|---|---|---|
| placebo | 570 | 111.2 | 0.95 | 30.0 | 111.0 | 150.0 | 4.62 | 3.39 | −80.0 | −0.86 | 1170 |
| 25 mg BID | 241 | 103.1 | 1.41 | 36.0 | 101.0 | 150.0 | −6.89 | 1.34 | −63.3 | −7.63 | 202.7 |

DAY 1 24 h

| | | | | | | | | | | | |
|---|---|---|---|---|---|---|---|---|---|---|---|
| placebo | 581 | 114.2 | 0.92 | 33.0 | 115.0 | 150.0 | 5.01 | 2.55 | −71.1 | 0.88 | 1190 |
| 25 mg BID | 253 | 100.9 | 1.27 | 58.0 | 98.0 | 150.0 | −9.69 | 1.13 | −38.3 | −11.30 | 159.5 |

DAY 21 anytime

| | | | | | | | | | | | |
|---|---|---|---|---|---|---|---|---|---|---|---|
| placebo | 554 | 117.5 | 1.43 | 10.0 | 117.0 | 711 | 8.73 | 3.21 | −92.3 | 2.27 | 1340 |
| 25 mg BID | 233 | 90.52 | 1.38 | 44.0 | 88.0 | 148.0 | −18.1 | 1.24 | −67.2 | −19.10 | 121.6 |

DAY 90 anytime

| | | | | | | | | | | | |
|---|---|---|---|---|---|---|---|---|---|---|---|
| placebo | 503 | 112.0 | 0.98 | 10.0 | 111.0 | 150.0 | 4.48 | 3.46 | −91.6 | −1.04 | 1320 |
| 25 mg BID | 223 | 91.05 | 1.54 | 29.0 | 89.0 | 150.0 | −17.7 | 1.90 | −72.1 | −20.16 | 305.4 |

Nominal time points were used for PD biomarker data analysis. For post-baseline time points, N represents subjects with a baseline and a post-baseline value for that time point. Values greater than the upper limit of quantification were set to the upper limit values for summary statistics. Baseline=Non-missing result with a collection date-time less than or equal to the date-time of the first active dose of study medication in each treatment group at time point “BASELINE—PRE DOSE”. The randomization of subjects to the 50 mg QD (N=22) and 100 mg QD (N=18) treatment arms was terminated with the implementation of revised protocol 05.

### Example 3. Bioavailability Study of 25 mg and 100 mg Film Coated DC Tablet Against 25 Mg and 100 mg Comparative SDP Oral Capsule in Healthy Participants and Results

The first Phase 1 trial is an open-label, randomized, crossover study to evaluate the relative oral bioavailability, pharmacokinetics, and food effect after single dose (for Part 1, Part 3, and Part 4) or multiple-dose (for Part 2). Part 1 of this first Phase 1 study is to evaluate the relative bioavailability and food effect of a single dose of 200 mg milvexian administered as film coated DC tablet compared with 100 mg comparative SDP oral capsule under fasting and fed conditions. Part 2 of this first Phase 1 study is to characterize the pharmacokinetic (PK) of multiple twice daily administered doses for 5 days of milvexian administered as 200 mg milvexian administered as film coated DC tablet and comparative SDP oral capsules at 25 mg or 200 mg. The 100 mg and 25 mg capsule formulations (see Table 7 below) are described in WO 2020210629 of which the capsule comprises MCC and lactose anhydrous DC in a weight ratio of 1:1 binder (MCC) to filler (lactose anhydrous). The composition and physical properties for the 25 mg and 100 mg film coated DC tablets are provided in the Table 1 above.

Blood samples were drawn at predetermined time points following drug administration as specified in the clinical study protocols. Concentration of the samples are measured using a validated analytical method (Liquid Chromatography with Tandem Mass Spectroscopy). Individual subject pharmacokinetic parameters (e.g. Cₘₐₓ, AUCₗₐₛₜ, and AUC_{∞}) are derived by non-compartmental methods using Phoenix™ WinNonlin® (version 8.1, Pharsight, A Certara™ Company, L.P., Princeton, NJ, USA) software from the time-concentration profiles.

TABLE 7
SDP Capsule Formulation Composition of 100 mg and 25 mg

| | 100 mg SDP | 25 mg SDP |
|---|---|---|
| Component | capsule | capsule |
| Intragranular | | |
| SDP of 75% Compound | 133.3 | 33.33 |
| (I): HPMCAS* | | |
| Microcrystalline cellulose | 167.7 | 41.925 |
| (MCC) | | |
| Lactose anhydrous DC | 167.7 | 41.925 |
| Weight ratio of MCC to lactose | 1:1 | 1:1 |
| anhydrous | | |
| Croscarmellose sodium | 12.5 | 3.125 |
| Colloidal silicon dioxide | 2.50 | 0.625 |
| Magnesium stearate | 1.25 | 0.3125 |
| Total Intragranular | 485 | 121 |
| Extragranular | | |
| Croscarmellose sodium | 12.5 | 3.125 |
| Magnesium stearate | 2.5 | 0.625 |
| Total | 500 | 125 |

*SDP prepared according to the composition and method described in WO 2020210629

The treatment regimens for Part 1 and Part 2 are summarized in Table 7 below.

TABLE 7
Treatment Regimen used in Part 1 and Part 2

| Treatment | Formulation | Dose | Food |
|---|---|---|---|
| A (Part 1A) | 100 mg film-coated DC* | 200 mg (2 × 100 mg) | Fasted |
| | oral tablet | | |
| C (Part 1A) | 100 mg SDP oral capsule | 200 mg (2 × 100 mg) | Fasted |
| D (Part 1B) | 100 mg film-coated DC* | 200 mg (2 × 100 mg) | Fed |
| | oral tablet | | |
| F (Part 1B) | 100 mg SDP oral capsule | 200 mg (2 × 100 mg) | Fed |
| G (Part 2A) | 100 mg film-coated DC* | 200 mg (2 × 100 mg) | BID for |
| | oral tablet | | 5 days |
| H (Part 2A) | 100 mg SDP oral capsule | 200 mg (2 × 100 mg) | BID for |
| | | | 5 days |
| I (Part 2B) | 25 mg film-coated DC* | 25 mg (1 × 25 mg) | BID for |
| | oral tablet | | 5 days |
| J (Part 2B) | 25 mg SDP oral capsule | 25 mg (1 × 25 mg) | BID for |
| | | | 5 days |

*DC: direct compression
**RC: roller compaction

The absolute Bioavailability for the 100 mg SDP capsule comparative formulation is 52% for fasted and 72% for fed conditions for a 200 mg dose.

In Part 1 of single dose administration regimen, 100 mg film-coated DC tablet shows about 9.0% to about 11% as compared to the oral SDP capsule at 200 mg dose. 100 mg film-coated DC* oral tablet has lower food effects. To achieve better patient compliance, it is preferred to have milveixan being administered with or without food. A drug formulation with small food effects provide better patient compliance.

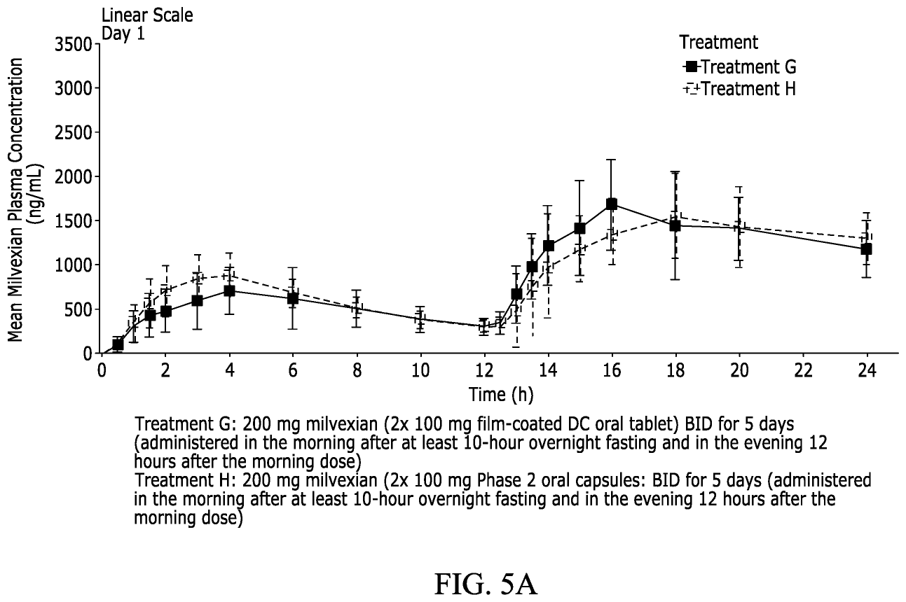

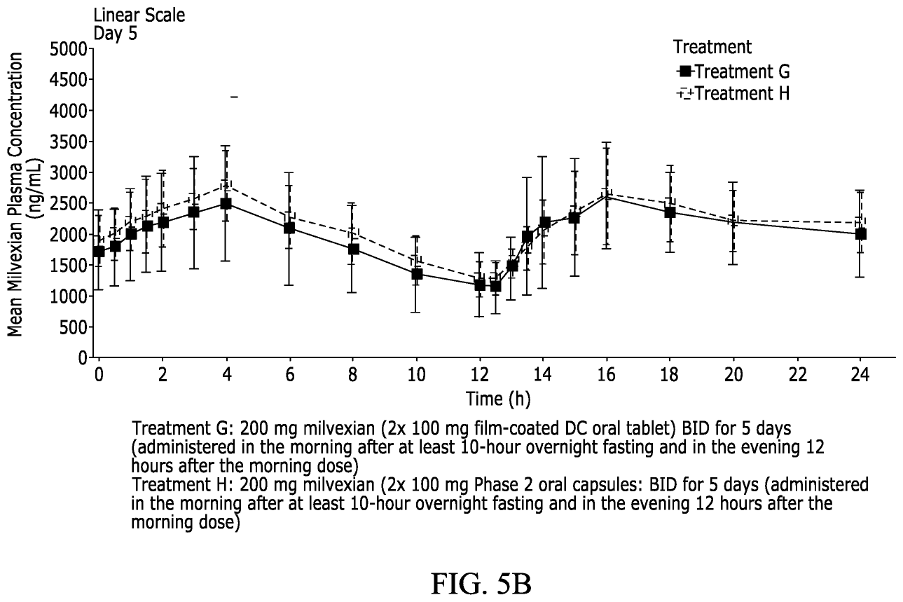

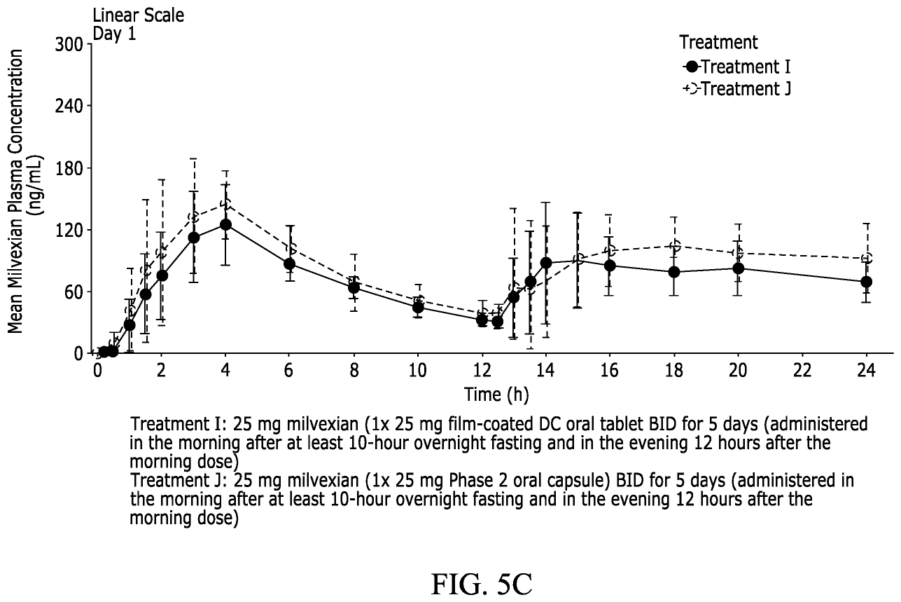

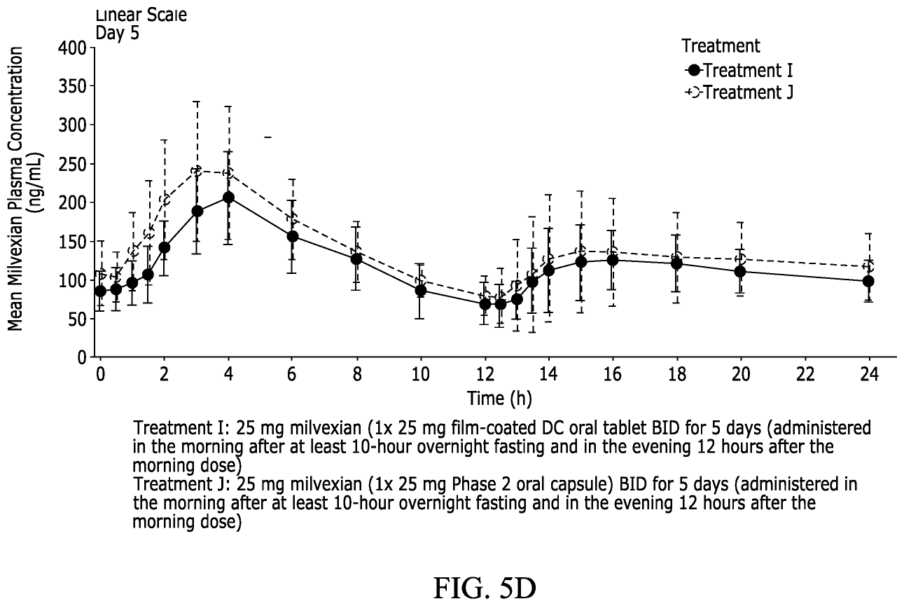

In Part 2 of multiple dose BID administration regimen, at 2×100 mg film-coated DC tablet shows about 5-700 lower bioavailability, as compared to the 2×100 mg SDP oral capsule. The 25 mg of the film-coated DC tablet (DC tablet) shows about 11-13% lower bioavailability, as compared to the 25 mg of SDP oral capsule) (See FIGS. 5A-5D for milvexian dosing curves as a function of time after BID administration).

In a second Phase 1 trial, Part 1 was an open-label, randomized, 3-way crossover study in healthy participants to evaluate the relative oral bioavailability, pharmacokinetics, and food effect of a single oral dose of 200 mg milvexian as 2×100 mg SDP DC tablet compared to 200 mg of 2×100 mg SDP granule capsule under fasting conditions and to assess the effect of food on the bioavailability of milvexian after a single dose of 200 mg milvexian as 2×100 mg SDP DC tablet. Part 2 was an open-label, randomized, 2-way crossover Study in healthy participants to evaluate the PK and relative bioavailability of a single oral dose of 50 mg milvexian as 2×25 mg SDP tablet compared to 50 mg milvexian as 2×25 mg SDP granule capsule in healthy participants under fasting conditions.

In the pooled analysis of PK (AUC_{inf} and Cₘₐₓ) data generated under the two phase 1 trials under fed and fasting conditions for 2×100 mg and 1×25 mg SDP DC tablets and 25 mg and 100 mg SDP granule capsule formulations, the results demonstrated that the tablet exhibited smaller inter-participant PK variability in healthy participants than those of capsule

The results of clinical studies demonstrated that, for tablets with similar dissolution rates, AUC_{∞} (also known as AUC_{inf}) of the 2×100 mg film-coated DC* oral tablet coated tablet relative to the 2×100 mg granule capsule formulations, met bioequivalence criteria.

### Example 4

Delayed cardiac repolarization is an undesirable side-effect of some non-antiarrhythmic drugs. Due to the potential clinical consequences of delayed cardiac repolarization, a rigorous characterization of a new pharmaceutical agent's ability to prolong QT/QTc interval was recommended.

Results from in vitro studies indicated that milvexian inhibited cardiac potassium (hERG/IKr) channel currents with weak to moderate potency at concentrations that greatly exceed unbound concentrations in the plasma of subjects treated with clinically relevant dose regimens of milvexian. The present study, which was conducted after the in vitro study results became available, evaluated the effect of milvexian on cardiac repolarization in healthy subjects, an important aspect of cardiovascular safety.

This placebo- and positive-controlled, TQT study was conducted to evaluate the effect of multiple-dose administration of milvexian on the QT/QTc interval duration and ECG morphology at therapeutic and supratherapeutic doses at steady state, in healthy adults.

| Objectives | Endpoints |
|---|---|
| Primary | |
| To assess the effects of milvexian on the | QTcF, change from |
| QT/QTcF intervals in healthy participants | baseline in QTcF |
| when administered at therapeutic (100 mg | (ΔQTcF), placebo- |
| twice daily as capsule) and supratherapeutic | corrected change from |
| (200 mg twice daily as solution) doses for 4 | baseline in QTcF |
| days. | (ΔΔQTcF). |
| Secondary | |
| To assess the effects of milvexian on other | HR, ΔHR, ΔΔHR, PR, |
| ECG parameters and ECG intervals in | ΔPR, ΔΔPR, QRS |
| healthy participants when administered at | duration, ΔQRS |
| therapeutic (100 mg twice daily as capsule) | duration, and ΔΔQRS |
| and supratherapeutic (200 mg twice daily as | duration. |
| solution) doses for 4 days. | |
| To assess the assay sensitivity by evaluating | QTc, baseline QTc, |
| the mean ΔQTc between moxifloxacin and | change from baseline |
| placebo (ΔΔQTc) on Day 4. | in QTc (ΔQTc), placebo- |
| | corrected change |
| | from baseline in QTc |
| | (ΔΔQTc) for QTcB |
| | and QTcP. |
| To assess the relationship between the | Characterization |
| systemic plasma concentrations of | of the milvexian |
| milvexian and QT/QTc changes. | concentration-ΔΔQTcF |
| | relationship. |

The upper bound of two-sided 9000 CI for the largest difference in mean change from baseline in QTc intervals (ΔQTc; based on the primary correction method—QTcF) between milvexian and placebo (ΔΔQTc) at the therapeutic (100 mg twice daily as capsule) and supratherapeutic (200 mg twice daily as solution) doses was less than 10 milliseconds.

A total of 66 participants were enrolled in the study and randomly assigned to 1 of 4 possible treatment sequences. All 66 (100.0%) participants enrolled in the study were included in the safety and PD analysis set: 55 participants each in the milvexian 100 mg and 200 mg groups, 58 participants in the moxifloxacin group, and 57 participants in the placebo group.

Pharmacokinetic analysis was performed on the plasma concentrations of each participant. Sixty-one participants were included in the PK analysis set, i.e., all randomized participants who received at least one dose of active study intervention whose PK profiles allowed for accurate calculation of at least one PK parameter. The PK/PD analysis was based on the participants included in the PD analysis set who had at least one measurement of milvexian concentration.

All 66 (100.0%) participants enrolled in the study received at least one entire dose of study intervention.

Forty-five (68.2%) participants completed all 4 periods with study intervention as planned. Per study intervention, 52 participants received milvexian 100 mg capsule twice daily for 4 days, 54 participants received milvexian 200 mg solution twice daily for 4 days, 58 participants received a single dose of 400 mg moxifloxacin, and 56 participants received placebo for 4 days.

There were no consistent or clinically relevant changes over time in mean vital signs.

No treatment-emergent actual values of QTcF or QTcB>480 ms or changes from baseline >60 ms were observed during the study. No ECG abnormalities were reported as TEAE in the study.

Milvexian exposure (i.e., Cₘₐₓ and AUC over a dosing interval) was approximately 2- to 3-times higher on Day 4, compared with Day 1, when 100 mg was administered twice daily as a capsule (Treatment A) and 200 mg was administered twice daily as a solution (Treatment B).

### Pharmacodynamic Results:

On administration of 100 and 200 mg milvexian, the upper limits of the 2-sided 90% CIs for ΔΔQTcF over the Day 1 and Day 4 postdose measurement intervals were below the protocol specified 10-ms limit at all timepoints (i.e., highest upper limits of 5.16 ms and 4.57 ms for 100 mg and 200 mg milvexian, respectively), showing there was no effect of clinical or regulatory concern on the QT interval in accordance with the ICH E14 guideline. These results were confirmed based on the ΔΔQTcF at milvexian Tₘₐₓ, i.e., the upper limits of the 90% CI were below 10 ms at the Tmax of milvexian after administration of 100 mg or 200 mg twice daily, from analyses that used milvexian Tₘₐₓ on Day 1 and Day 4 (combined) or milvexian Tₘₐₓ on Day 1 and Day 4 separately.

Study-specific and Bazett's correction methods also showed there was no effect of clinical or regulatory concern on the QT interval in accordance with the ICH E14 guideline. Assay sensitivity for moxifloxacin was also demonstrated when using Bazett's or the study-specific power correction methods for HR.

No treatment-emergent actual values of QTc>480 ms were observed during the study. No treatment-emergent changes from baseline of QTcF or QTcP>60 ms were observed during the study. One (1.7%) participant had a treatment-emergent change from baseline of QTcB>60 ms during the study (i.e., following moxifloxacin).

No consistent or clinically relevant changes over time were observed in HR, RR interval, PR interval, or QRS width. No clinically meaningful differences were observed between treatment groups.

### Pharmacokinetics/Pharmacodynamics Results:

Based on the results of linear mixed effects modeling, no statistical significant relationship was observed between milvexian concentration and ΔΔQTcF (p=0.8454), ΔΔQTcP (p=0.7102), or ΔΔQTcB (p=0.8670). At Cₘₐₓ, no statistically significant effect of treatment (100 or 200 mg milvexian) on ΔΔQTc was observed (p>0.5).

### CONCLUSIONS

No notable study limitations were identified by the sponsor.

Administration of multiple doses of 100 and 200 mg milvexian was generally safe and well tolerated in healthy adult participants.

The upper limits of all 2-sided 90% CIs of the time-matched differences between 100 mg milvexian and placebo and between 200 mg milvexian and placebo in changes from baseline in QTcF were below 10 ms. Hence, milvexian at therapeutic (100 mg) and supratherapeutic (200 mg) doses does not show evidence of QT/QTc interval prolongation of clinical or regulatory concern, according to the ICH E14 guideline.

Assay sensitivity was demonstrated by moxifloxacin as the positive control.

Based on the results of linear mixed effects modeling, no statistical significant relationship was observed between milvexian concentration and ΔΔQTcF (p=0.8454).

1. A method of preventing adverse cerebrovascular events or adverse cardiovascular events in a human patient with acute coronary syndrome, wherein the method comprises administering to the human patient a regimen comprising: (i) a pharmaceutical composition comprising 25 mg of milvexian (or pharmaceutically acceptable salt or solvate thereof); and (ii) an antiplatelet therapy selected from the group consisting of aspirin, a P2Y12 inhibitor, and combination thereof; wherein the pharmaceutical composition is administered twice daily.

2. The method of claim 1, wherein the antiplatelet therapy is a P2Y12 inhibitor.

3. The method of claim 2, wherein the P2Y12 inhibitor is clopidogrel, ticagrelor, or prasugrel.

4. The method of claim 1, wherein the antiplatelet therapy is aspirin.

5. The method of claim 1, wherein the human patient is treated with aspirin and clopidogrel combination therapy from day 1 to day 21, followed by aspirin monotherapy for at least 90 days.

6. A method for preventing adverse cardiovascular events in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof;
wherein the pharmaceutical composition is administered twice daily; and
wherein the adverse cardiovascular event is one or more selected from the group consisting of all-cause mortality (ACM); cardiovascular (CV) death; myocardial infarction (MI); unstable angina (UA); “all stroke” or “any stroke” (ischemic, hemorrhagic, or unknown cause); ischemic stroke; acute limb ischemia (ALI); major vascular (non-traumatic) limb amputation; symptomatic venous thromboembolism (VTE: (pulmonary embolism (PE), deep vein thrombosis (DVT)); ischemia-driven coronary revascularization; stent thrombosis; hospitalization for any reason, categorized as (1) planned or unplanned, (2) for arterial or venous thrombotic event or neither; and transient ischemic attack (TIA).

7. The method of claim 6, wherein the adverse cardiovascular event is one or more of CV death, MI, or ischemic stroke.

8. A method for reducing the incidence rate of one or more adverse thrombotic events selected from ischemic stroke, myocardial infarction (MI), or all-cause death in a human patient diagnosed with acute coronary syndrome, wherein the method comprises administering to the human patient a regimen comprising: (i) a pharmaceutical composition comprising about 25 mg to about 100 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof; wherein the pharmaceutical composition is administered twice daily.

9. A method for preventing ischemic stroke in a human patient diagnosed with acute coronary syndrome, wherein the method comprises administering to the human patient a regimen comprising: (i) a pharmaceutical composition comprising 25 mg of milvexian (or pharmaceutically acceptable salt or solvate thereof) and one or more pharmaceutically acceptable excipients, and (ii) an antiplatelet therapy selected from the group consisting of aspirin, a P2Y12 inhibitor, and combination thereof, wherein the pharmaceutical composition is administered twice daily.

10. The method of claim 8, wherein the clinical benefit of reducing the incidence rate of the ischemic stroke by the regimen is maintained throughout a treatment period of 90 days.

11. The method of claim 1, wherein administration of the regimen reduces factor XI FXI) clotting activity by about 7% to about 20% relative to baseline of the patient.

12. The method of claim 1, wherein administration of the regimen results in a prolongation of activated partial thromboplastin time (aPTT) ranging from about 27% to about 64% relative to baseline.

13. The method of claim 1, where the administration does not result in a statistically significant increase in major bleeding complications.

14. The method of claim 1, wherein the pharmaceutical composition comprising milvexian (or pharmaceutically acceptable salt or solvate thereof) is a solid oral pharmaceutical composition.

15. The method of claim 1, wherein the administration results in a milvexian plasma half-life ranging from about 13 hours to about 16 hours.

16. The method of claim 1, wherein the administration results in a milvexian plasma concentration reaching steady-state at about 3 days to 6 days.

17. The method of claim 1, wherein the pharmaceutical composition comprising milvexian (or pharmaceutically acceptable salt or solvate thereof) is administered without regard to the timing of food intake.

18. The method of claim 1, wherein the method results in no clinically significant corrected QT (QTc) interval prolongation.
